SEQUENCE LISTING (75 sequences)

SEQ ID NO: 1 - DNA, 56 bp, Artificial Sequence
  Description of Artificial Sequence Synthetic oligonucleotide
  modified_base (22)..(29): a, c, t, g, unknown or other
  modified_base (46)..(46): 3-Cyanovinylcarbazole
acgagcatcagcagcatacgannnnnnnnttgatgaattcatcaangttaagttgt

SEQ ID NO: 2 - DNA, 56 bp, Artificial Sequence
  Description of Artificial Sequence Synthetic oligonucleotide
  modified_base (22)..(29): a, c, t, g, unknown or other
  modified_base (46)..(46): 3-Cyanovinylcarbazole
acgagcatcagcagcatacgannnnnnnnttaggtttaaacctaanatgatgatgt

SEQ ID NO: 3 - DNA, 51 bp, Artificial Sequence
  Description of Artificial Sequence Synthetic oligonucleotide
  modified_base (41)..(41): 3-Cyanovinylcarbazole
catcatcattttaggttttgtggtttgatgaattcatcaangttaagttgt

SEQ ID NO: 4 - DNA, 51 bp, Artificial Sequence
  Description of Artificial Sequence Synthetic oligonucleotide
  modified_base (41)..(41): 3-Cyanovinylcarbazole
caacttaactttgatgaatgtggtttaggtttaaacctaanatgatgatgt

SEQ ID NO: 5 - DNA, 51 bp, Artificial Sequence
  Description of Artificial Sequence Synthetic oligonucleotide
  modified_base (41)..(41): 3-Cyanovinylcarbazole
catcatcattttaggtttaataagttgatgaattcatcaangttaagttgt

SEQ ID NO: 6 - DNA, 51 bp, Artificial Sequence
  Description of Artificial Sequence Synthetic oligonucleotide
  modified_base (41)..(41): 3-Cyanovinylcarbazole
caacttaactttgatgaagattttttaggtttaaacctaanatgatgatgt

SEQ ID NO: 7 - DNA, 51 bp, Artificial Sequence
  Description of Artificial Sequence Synthetic oligonucleotide
  modified_base (41)..(41): 3-Cyanovinylcarbazole
catcatcattttaggtttgttagattgatgaattcatcaangttaagttgt

SEQ ID NO: 8 - DNA, 51 bp, Artificial Sequence
  Description of Artificial Sequence Synthetic oligonucleotide
  modified_base (41)..(41): 3-Cyanovinylcarbazole
caacttaactttgatgaagaggaattaggtttaaacctaanatgatgatgt

SEQ ID NO: 9 - DNA, 39 bp, Artificial Sequence
  Description of Artificial Sequence Synthetic oligonucleotide
agagacagattgcgcaatgtttttttttttttttttttt

SEQ ID NO: 10 - DNA, 45 bp, Artificial Sequence
  Description of Artificial Sequence Synthetic oligonucleotide
caacttaactttgatgaaatcaaaaaaaaaaaaaaaaaaaaaaaa

SEQ ID NO: 11 - DNA, 26 bp, Artificial Sequence
  Description of Artificial Sequence Synthetic oligonucleotide
  modified_base (17)..(17): 3-Cyanovinylcarbazole
ttgatgaattcatcaangttaagttg

SEQ ID NO: 12 - DNA, 26 bp, Artificial Sequence
  Description of Artificial Sequence Synthetic oligonucleotide
  modified_base (17)..(17): 3-Cyanovinylcarbazole
ttaggtttaaacctaanatgatgatg

SEQ ID NO: 13 - DNA, 18 bp, Artificial Sequence
  Description of Artificial Sequence Synthetic oligonucleotide
catcatcattttaggttt

SEQ ID NO: 14 - DNA, 18 bp, Artificial Sequence
  Description of Artificial Sequence Synthetic oligonucleotide
caacttaactttgatgaa

SEQ ID NO: 15 - DNA, 122 bp, Artificial Sequence
  Description of Artificial Sequence Synthetic polynucleotide
  modified_base (22)..(31): a, c, t, g, unknown or other
  modified_base (92)..(101): a, c, t, g, unknown or other
gtatcgtgcaagggtgaatgcnnnnnnnnnntttgagggaaccagctactagatggttcgattatattggtataaaaactcaaactaatctnnnnnnnnnncattgccgtatgggctaggat

SEQ ID NO: 16 - DNA, 122 bp, Artificial Sequence
  Description of Artificial Sequence Synthetic polynucleotide
  modified_base (1)..(1): a, c, t, g, unknown or other
ntatcgtgcaagagtgaatgcgttgcactcgtttgagggaaccagctacttgatggttcgattatattggtatattaactcattctaatcttaagtctttccattgccgtatgggctaggat

SEQ ID NO: 17 - DNA, 26 bp, Artificial Sequence
  Description of Artificial Sequence Synthetic primer
  modified_base (20)..(26): a, c, t, g, unknown or other
tgttaagttgtttgatgaannnnnnn

SEQ ID NO: 18 - DNA, 178 bp, Artificial Sequence
  Description of Artificial Sequence Synthetic polynucleotide
gtatcgtgcaagggtgaatgcaattattgatgaaaagggggacagtgccaggtggggagtttgactggggcggtacacctgtcaaacggtaacgcaggtgtcctaaggcgagctcagggaggacagaaaaaaaaaaaaaaaaaaaaaaaggtacccacattgccgtatgggcgaggat

SEQ ID NO: 19 - DNA, 202 bp, Artificial Sequence
  Description of Artificial Sequence Synthetic polynucleotide
gtatcgtgcaagggtgaatgcaattattgatgaaatgggttgtgcttcacttctgcggtaaccctgtggactttccctccatagaaaggcttcgtgtggaatatcactgtcgctgagtacccagtcttggcacagttgatgctgacttttcaaagaaaaaaaaaaaaaaaagaaagccgctcattgccgtatgggctaggat

SEQ ID NO: 20 - DNA, 166 bp, Artificial Sequence
  Description of Artificial Sequence Synthetic polynucleotide
gtatcgtgcaagggtgaatgcaattattgatgaaggacccgtggtcaccatggtaggcacggcgactaccatcgaaagttgatagggcagacgttcgaatgggtcgtcgccgccacggaaaaaaaaaaaaaaaaatgatacaccgcattgccgtatgggctaggat

SEQ ID NO: 21 - DNA, 27 bp, Artificial Sequence
  Description of Artificial Sequence Synthetic primer
  modified_base (20)..(24): a, c, t, g, unknown or other
tgttaagttgtttgatgaannnnnggg

SEQ ID NO: 22 - DNA, 174 bp, Artificial Sequence
  Description of Artificial Sequence Synthetic polynucleotide
gtatcgtgcaagggtgaatgcaattattgatgaagcagcgggatcagaacagtactgcatttacttgccaacagaacagacagacctgaagtcaagacaactgcattctctgtgaagtctgtaaaaaaaaaaaaaaaaaaaaaatggcaccaccaatgccgtatgggctaggat

SEQ ID NO: 23 - DNA, 167 bp, Artificial Sequence
  Description of Artificial Sequence Synthetic polynucleotide
gtatcgtgcaagggtgaatgcaattattgatgaaggcatgggaatcgcgtgaacccaggaggcggaggttgcagtgagccgagatcgcgccactgcactccaagctgggggaccgaacgaaaaaaaaaaaaaaaaaacgttaatctcatttccgtatgggctaggat

SEQ ID NO: 24 - DNA, 223 bp, Artificial Sequence
  Description of Artificial Sequence Synthetic polynucleotide
gtatcgtgcaagggtgaatgcaattattgatgaagcggcgggagataccccgacagtgtgggcagtttcatttcagcagcaatcaagagggggcctggagccactcaatcaagtggagcaggatgggagcaagcactgtgcagaccaatgcaatccccagttaacacaaaaaaaagaaaaaaaaaaaaaagatacagtgaaccattgccgtatgggcgaggat

SEQ ID NO: 25 - DNA, 27 bp, Artificial Sequence
  Description of Artificial Sequence Synthetic primer
  modified_base (20)..(24): a, c, t, g, unknown or other
tgttaagttgtttgatgaannnnnttt

SEQ ID NO: 26 - DNA, 165 bp, Artificial Sequence
  Description of Artificial Sequence Synthetic polynucleotide
gtatcgtgcaagggtgaatgcaattattgatgaaccttgtttgtggcatatcttcaggtggcccaaaccggaatctagggacaccagcaacctgcggcgaatgaattgcttaaaaaaaaaaaaaaaaaaaaaaatgccaacccccattgccgtatgggctaggat

SEQ ID NO: 27 - DNA, 226 bp, Artificial Sequence
  Description of Artificial Sequence Synthetic polynucleotide
gtatcgtgcaagggtgaatgcaattattgatgaatgatcttttcaccgtgccagactagagtcaagctcaacagggtcttcttttcccgctgattccgccaagcccgttcccttggctgtggtttcgctggatagtaaaaaaaaaaaaaaaaactagttctcccattgccgtctgggctaggattgtatcgtgcaagggtgaatgcattgccgtatgggctaggat

SEQ ID NO: 28 - DNA, 172 bp, Artificial Sequence
  Description of Artificial Sequence Synthetic polynucleotide
gtatcgtgcaagggtgaatgcaattattgatgaaggaactttggaaccgagctggtgtcagtggtggtggtggccagcttccccagaaagctccatgatccgctcatctggcgttgcactcaaaaaaaaaaaaaaaaaaaagctgctcgcacattgccgtatgggcgaggat

SEQ ID NO: 29 - DNA, 44 bp, Artificial Sequence
  Description of Artificial Sequence Synthetic primer
tcgtcggcagcgtcagatgtgtataagagacagattgcgcaatg

SEQ ID NO: 30 - DNA, 21 bp, Artificial Sequence
  Description of Artificial Sequence Synthetic primer
acgagcatcagcagcatacga

SEQ ID NO: 31 - DNA, 27 bp, Artificial Sequence
  Description of Artificial Sequence Synthetic primer
  modified_base (20)..(24): a, c, t, g, unknown or other
tcaacttaactttgatgaannnnnggg

SEQ ID NO: 32 - DNA, 190 bp, Artificial Sequence
  Description of Artificial Sequence Synthetic polynucleotide
  modified_base (22)..(29): a, c, t, g, unknown or other
acgagcatcagcagcatacgannnnnnnnttgatgaagaggaattaggtttgttagattgatgaagattttttaggtttaataagttgatgaatgtggtttaggttttgtggtttgatgaaatcaaaaaaaaaaaaaaaaaaaaaacattgcgcaatctgtctcttatacacatctgacgctgccgacga

SEQ ID NO: 33 - DNA, 192 bp, Artificial Sequence
  Description of Artificial Sequence Synthetic polynucleotide
acgagcatcagcagcatacgacccttggcttgatgaagaggaattaggtttgttagattgatgaagattttttaggtttaataagttgatgaatgtggtctaggttttgtggtttgatgaaatcaaaaaaaaaaaaaaaaaaaaaaaaacattgcgcaatctgtctcttatacacatctgacgctgcgacga

SEQ ID NO: 34 - DNA, 249 bp, Artificial Sequence
  Description of Artificial Sequence Synthetic polynucleotide
acgagcatcagcagcatacgaaaaaaggcttgatgaagaggaattaggtttgttagattgatgaagattttttaggtttaataagttgatgaatgtggtttaggttttgtggtttgatgaaatcaaaaaaaaaaaaaaaaaaacattgcgcaatcatagtcttatacacaaaagacgcttccagaacggaacagcaaaagtatgaacaccagacaatgataaagatgacggaatccctatacagatgta

SEQ ID NO: 35 - DNA, 250 bp, Artificial Sequence
  Description of Artificial Sequence Synthetic polynucleotide
acgagcatcagcagcatacgatagccgctttgatgaagaggaattaggtttgttagattgatgaagattttttaggtttaataagttgatgaatgtggtttaggttttgtggtttgatgaaatcaaaaaaaaaaaaaaaaaaaaaaaaaaaaaaaaaatgggcaaaattgtggagataaaaaacagtcccgggccacacttagggatatcaaaccgcttaacaccactccctgaacaccattcggtttgt

SEQ ID NO: 36 - DNA, 130 bp, Artificial Sequence
  Description of Artificial Sequence Synthetic polynucleotide
acgagatcagcagcatacgatgagaggattgatgaagattttttaggtttaataagttgatgaatgtggtttaggttttgtggtttgatgaaatcaaaaaaaaaaaaaaaaaaaattgcgcaatctgtct

SEQ ID NO: 37 - DNA, 150 bp, Artificial Sequence
  Description of Artificial Sequence Synthetic polynucleotide
acgagcatcagcagcatacgatagaggggttgatgaagaggaattaggtttgttagattgatgaatgtggtttaggttttgtggtttgatgaaatcaaaaaaaaaaaaaaaaaaaaaacattgcgcaatatttctattaaacacaactga

SEQ ID NO: 38 - DNA, 118 bp, Artificial Sequence
  Description of Artificial Sequence Synthetic polynucleotide
acgagcatcagcagcatacgagactggggttgatgaagaggaattaggtttgttagattgatgaaatcaaaaaaaaaaaaaaaaaaaaaacattgcgcaatctgtctcttatacacat

SEQ ID NO: 39 - DNA, 134 bp, Artificial Sequence
  Description of Artificial Sequence Synthetic polynucleotide
acgagcatcagcagcatacgaatctcttgttgatgaaggttttttaggtttaataagttgatgaaatcaaaaaaaaaaaaaaaaaaaaacattgcgcaatctgtctcttatacacatctgacgctgccgaccga

SEQ ID NO: 40 - DNA, 140 bp, Artificial Sequence
  Description of Artificial Sequence Synthetic polynucleotide
acgagcatcagcagcatacgattggcacgttgatgaatgtggtttaggttttgtggtttgatgaataactgggaaaaaaaaaaaaaaaaaaaaaaaaacattgcgcaatctgtctcttatacacatctgacgctgccgac

SEQ ID NO: 41 - DNA, 132 bp, Artificial Sequence
  Description of Artificial Sequence Synthetic polynucleotide
acgagcatcagcagcatacgagttgagttttgatgaatgtggtctaggttttgtggtatgatgagtataggggaaaaaaaaaaaaaaaaacattgcgcaatctgtctcttatacacatctgacgctgccgac

SEQ ID NO: 42 - DNA, 113 bp, Artificial Sequence
  Description of Artificial Sequence Synthetic polynucleotide
acgagcatcagcagcatacgaagaccatgttgatgaatgtggtttaggttttgtggtttgatgaaatctcgggaaaaaaaaaaaaaaacattgcgcaatctgtctcttataca

SEQ ID NO: 43 - DNA, 200 bp, Artificial Sequence
  Description of Artificial Sequence Synthetic polynucleotide
acgagcatcagcagcatacgagagtgtccttgatgaagattttttaggttttgtggtttgatgaatgcatggtttaaaagagagaaaaggaaaaaagacacaaagctggttacaggtctgagggagtctaaggagagaaaaaaaaaaaaaaaaaaaaaaattgcgcaatctgtctcttatacacatctgacgctgccgac

SEQ ID NO: 44 - DNA, 148 bp, Artificial Sequence
  Description of Artificial Sequence Synthetic polynucleotide
acgagcatcagcagcatacgagaagtattttgatgaagattttttaggttttgtggtttgatgaaggcacgggtcgggagtgggtaagaaaaaaaaaaaaaaaaacattgcgcaatctgtctcttatacacaactgacgctgcccgac

SEQ ID NO: 45 - DNA, 148 bp, Artificial Sequence
  Description of Artificial Sequence Synthetic polynucleotide
acgagcatcagcagcatacgataaggtagttgatgaagattttttaggttttgtggtttgatgaaaatgaggtttcccgtgttgggtcaaaaaaaaaaaaaaaaacattgcgcaatctgtctcttatacacaacttacgctgccgaca

SEQ ID NO: 46 - DNA, 250 bp, Artificial Sequence
  Description of Artificial Sequence Synthetic polynucleotide
acgagcatcagcagcatacgatcggagacatgatgaagaggaattaggttttgtggtttgatgaacatccgggagttggaggttgtggtaagctgagatcacagcattgcactccagcctgggcaacaagagcgaaaactccgtctcaaaacaaaacaaaaaaaccttaagctgggtgcggttgctcacgcctgtcatcccagcaccttgggaggaaaaaaaaaaaaaaaaaacattgagcaatctgtct

SEQ ID NO: 47 - DNA, 177 bp, Artificial Sequence
  Description of Artificial Sequence Synthetic polynucleotide
acgagcatcagcagcatacgatggtgatcttgatgaagaggaattaggttttgtggtttgatgaaattccgggtggctgaacgccacttgtccctctaagaagttgggggacgccgaccgctcgggggtcgcgtaactagttagcatgccagagtctcgttcgttatcggaattaac

SEQ ID NO: 48 - DNA, 159 bp, Artificial Sequence
  Description of Artificial Sequence Synthetic polynucleotide
acgagcatcagcagcatacgagagggactttgatgaagaggaattaggttttgtggtttgatgaatttgtgggtccatttgcattgcagggtctttgtaaaaaaaaaaaaaaaaaaaacattgcgcaatctgtctctatacacatctgacgctgccgac

SEQ ID NO: 49 - DNA, 207 bp, Artificial Sequence
  Description of Artificial Sequence Synthetic polynucleotide
acgagcatcagcagcatacgaagtagtcattgatgaatgtggtttaggtttaataagttgatgaattctcgggtaagtgcgggtcataagcttgcgttgattaagtccctgcccttgtacacaccgcccgtcgctactaccgattggatggtttagtgagaaaaaaaaaaaaaaaaaaacattgcgcaatctgtctcttatacacag

SEQ ID NO: 50 - DNA, 225 bp, Artificial Sequence
  Description of Artificial Sequence Synthetic polynucleotide
acgagcatcagcagcatacgagcaatctcttgatgaatgtggtttaggtttaattagttgatgaaactggggggggtggaggttgcagtgggctgagatcacaccactgtactccagcctggacaacagagccactgcactccagcctgggcaacagagagagactccatcttaaaaaaaaaaaaaaaaaaaacattgcgcaatctgtctcttatacacatctga

SEQ ID NO: 51 - DNA, 152 bp, Artificial Sequence
  Description of Artificial Sequence Synthetic polynucleotide
acgagcatcagcagcatacgatcgtgggtttgatgaatgtggtttaggtttaataagttgatgaatgaatgggctgatggtttcgataataactagtatgggaaaaaaaaaaaaaaaaaaaacattgcgcaaactgtcgcttatacacatct

SEQ ID NO: 52 - DNA, 152 bp, Artificial Sequence
  Description of Artificial Sequence Synthetic polynucleotide
acgagcatcagcagcatacgagtaaaaagttgatgaagattttttaggtttaataagttgatgaattcacgggtcgggagtgggtaatgataaaaaaaaaaaaaaaaaaaaaaacattgcgcaatctgtctcttatacacatctgacgctgc

SEQ ID NO: 53 - DNA, 250 bp, Artificial Sequence
  Description of Artificial Sequence Synthetic polynucleotide
acgagcatcagcagcatacgacatggagattgatgaagattttttaggtttaataagttgatgaatagatggggaagcatgtaacgcagaagaggactgacatcgtaagtttcagaaatcccatgaagtattgtaataagatcaggaacattacataagtgagggaatggcttttaaaaaaaaaaaaaaaaacattgcgcaaaatgtctcttataaacatcttacatataggagagaaaacggatgaact

SEQ ID NO: 54 - DNA, 181 bp, Artificial Sequence
  Description of Artificial Sequence Synthetic polynucleotide
acgagcatcagcagcatacgagaatgtggttgatgaagattttttaggtttaataagttgatgaatgctagggtgacttcatatgagattgtttgggctactgctcgcagtgcgccaatcaaaaaaaaaaaaaaaaaacattgcgcaatctgtctcttatacacatctgacgctgccgaca

SEQ ID NO: 55 - DNA, 161 bp, Artificial Sequence
  Description of Artificial Sequence Synthetic polynucleotide
acgagcatcagcagcatacgagctgttggttgatgaagaggaattaggtttaataagttgatgaaatagggggtttatttattaatatatgatagccttggctcaaaaaaaaaaaaaaaaaaacattgcgcaatctgtctcttatacacatctgacgctgc

SEQ ID NO: 56 - DNA, 227 bp, Artificial Sequence
  Description of Artificial Sequence Synthetic polynucleotide
acgagcatcagcagcatacgacgcgagttttgatgaagaggaattaggtttaataagttgatgaatatgagggtggtgattagtcggttgttgatgagatatttggaggtggggatcaatagagggggaaatagaatgatcagtactgcggcgggtaggcctaggaaaaaaaaaaaaaaaaaacattgcgcaatctttcacttataaacatcttacgctgccgacga

SEQ ID NO: 57 - DNA, 191 bp, Artificial Sequence
  Description of Artificial Sequence Synthetic polynucleotide
acgagcatcagcagcatacgattgcttcgttgatgaagaggaattaggtttaataagttgatgaactcagggatgcccctgttgagggttttggtggcctcattggctcctttccgaagctgcttatagttaaaaaaaaaaaaaaaaacattgcgcaatctgtctcttataaaaatctgacgctgccgacg

SEQ ID NO: 58 - DNA, 249 bp, Artificial Sequence
  Description of Artificial Sequence Synthetic polynucleotide
acgagcatcagcagcatacgaacctttttttgatgaatgtggtttaggtttgttagattgatgaataacaggggactatttaatccatctaaaaatacaaatcaggaaatggggggaaccataggaaaatcctccacctctaacagagcgaagttactggctttctgcttgctccaagaatcccaaggcttgatgtttggaaggaattataaaaaaaaaaaaaaaaaaacattgcgcaatctgtctctt

SEQ ID NO: 59 - DNA, 181 bp, Artificial Sequence
  Description of Artificial Sequence Synthetic polynucleotide
acgagcatcagcagcatacgatttgatctttgatgaatgtggtttaggtttgttagattgatgaaacagcggttatccaagtaggagaggagcgagcgaccaaaggaaccataactgatttaaaaaaaaaaaaaaaaaaacattgcgcaatctgtctcttatacacagctgacgctgccga

SEQ ID NO: 60 - DNA, 236 bp, Artificial Sequence
  Description of Artificial Sequence Synthetic polynucleotide
acgagcatcagcagcatacgaaagtggtattgatgaatgtggtttaggtttgttagattgatgaagacatggtaatcctgctcagtacgagaggaaccgcaggttcagacatttggtgtatgtgcttggctgaggagccaatggggcgaagctaccatctgtgggattatgaaaaaaaaaaaaaaaaaaaaaacattgcgcaatctgtctcttatacacatctgacgctgcgacga

SEQ ID NO: 61 - DNA, 250 bp, Artificial Sequence
  Description of Artificial Sequence Synthetic polynucleotide
acgagcatcagcagcatacgagcgcattattgatgaagattttttaggtttgttagattgatgaattcgggggtgcgtcgggtctgcgagagcgccagctatcctgagggaaacttcggagggaaccagctactagatggttcgattagtctttcgcccctatacccaggtcggacgaccgatttgaacgtcaggaccgctacggacctccaccagagtttcctctggcttcgccctgcccaggcatagt

SEQ ID NO: 62 - DNA, 196 bp, Artificial Sequence
  Description of Artificial Sequence Synthetic polynucleotide
acgagcatcagcagcatacgacgggcgagttgatgaagattttttaggtttgttagattgatgaaatgacgggtggatcacttgaagtcaggagtttgagaccagcctgtccaacataatcaaaccctgtctctactaaaaaaaaaaaaaaaaaaaaaaattgcgcaatctgtctcttatacacatatgacgctgc

SEQ ID NO: 63 - DNA, 249 bp, Artificial Sequence
  Description of Artificial Sequence Synthetic polynucleotide
acgagcatcagcagcatacgagaaattctttgatgaagattttttaggtttgttagattgatgaagcactgtgaaaccaaaatattcatgtaacttgctttatttagtgttactcgctttattgtggtggcttggaaccaaacctacaatatctgtatatatctaaacatgctaaaaaaaaaaaaaaaaaaaacattgctaaacctagccccaaacccactccaccttactaccagacaaccttagcca

SEQ ID NO: 64 - DNA, 152 bp, Artificial Sequence
  Description of Artificial Sequence Synthetic polynucleotide
acgagcatcagcagcatacgactccacatataggtttaataagttgatgatacgcggataaaactgcgtggcgggggtgcgtcgggtctgcgagagcgccagctatcctgagggaaacttcggagggaaccagctactagatggttcgatta

SEQ ID NO: 65 - DNA, 160 bp, Artificial Sequence
  Description of Artificial Sequence Synthetic polynucleotide
acgagcatcagcagcatacgaagcttcccttaggtttaataagttgatgaaatgtcggcgtaaagagtgttttagatcgccccctccccaataaagaaaaaaaaaaaaaaaaaaaaaacattgcgcaagctgtctcttataaacatctgacgctgccacg

SEQ ID NO: 66 - DNA, 249 bp, Artificial Sequence
  Description of Artificial Sequence Synthetic polynucleotide
acgagcatcagcagcatacgagctcgtttttaggtttaataagttgatgaagaagggggcacatccctcccactggcatcggttcctaccccacaaggggaaagtgagatcttccagttaggagcaatttggtgaaaggagtcgggtgaccagagctggaataaccgtctgcatggctgcactctggtatcagagtggagacagaagtaaggatagtgtggagggcacagttgcaagttgagtctttcc

SEQ ID NO: 67 - DNA, 249 bp, Artificial Sequence
  Description of Artificial Sequence Synthetic polynucleotide
acgagcatcagcagcatacgatgcgtcgtttaggttttgtggtttgatgaacaggcgggtgggttcctgctgtactttggctttacatctttcccctgccctcccatttctcccaacagcttgatgcagctattggcctgccagaggggatgtaatgcccagaccaactttttattgagctgatgcagctaacagacttgtaaacagtgccagtcgacaaagttttgctgaatattacagcatgtgccc

SEQ ID NO: 68 - DNA, 157 bp, Artificial Sequence
  Description of Artificial Sequence Synthetic polynucleotide
acgagcatcagcagcatacgattgcaatgttaggttttgtggtttgatgaacatacgggatgctgaaatgtgtacatgtgctaaaagagatatgtaggaaaaaaaaaaaaaaaaacattgcgcaatctgtctcttatacaaatcttacgctgccgac

SEQ ID NO: 69 - DNA, 176 bp, Artificial Sequence
  Description of Artificial Sequence Synthetic polynucleotide
acgagcatcagcagcatacgatggctcgcttaggttttgtggtttgatgaacctcagggttactagtttgtatctgtcagtatcatcattacgatgaggatgataaccaataaaaaaaaaaaaaaaaaaaaaaacattgcgcaatctgtctcttatacacatctgacgctgccgac

SEQ ID NO: 70 - DNA, 249 bp, Artificial Sequence
  Description of Artificial Sequence Synthetic polynucleotide
acgagcatcagcagcatacgagctgacgattaggtttgttagattgatgaaaagacgggtcgggtgggtatccgacgtcgcccccgacccccggcgcgcgcgccgcccgcccccacatcgggggacgcgcgccggggccccacagaaccaacccccggccccccggcccgaccccgccacccccccaaagtcaacccggaacctacggggaagttgcaaaggacctttatgagagtggtcacgacaaaa

SEQ ID NO: 71 - DNA, 174 bp, Artificial Sequence
  Description of Artificial Sequence Synthetic polynucleotide
acgagcatcagcagcatacgagaaatgtattaggtttgttagattgatgaatttgcgggatgcagcggcgcgcgggctttggtcggtccgggggtcgtttcgcctcttcttcacactgctccagaaaaaaaaaaaaaaaaaaaaaaattgcgcaatctgtctcttatacacatc

SEQ ID NO: 72 - DNA, 249 bp, Artificial Sequence
  Description of Artificial Sequence Synthetic polynucleotide
acgagcatcagcagcatacgatgcgaggcttaggtttgttagattgatgaaccgtggggtctgatgagcgtcggcatcgggcgccttaacccggcgttcggttcatcccgcagcgccagttctgcttaccaaaagtggcccactaggcactcgcattccacgcccggctccacgccagcgagccgggcttcttacccatttaaagtttgagaataggttgagatcgtttcggccccactacctctaatc

SEQ ID NO: 73 - DNA, 163 bp, Artificial Sequence
  Description of Artificial Sequence Synthetic polynucleotide
acgagcatcagcagcatacgaggctcgttttaggttttgtggtttgatgaaggtaaatagaaaaacaggttttaatatagtcagataatgcataaacacaaaaaaaaaaaaaaaaaaaaaaacattgcgcaatctgtctcttatacacaactgacgctgccga

SEQ ID NO: 74 - DNA, 191 bp, Artificial Sequence
  Description of Artificial Sequence Synthetic polynucleotide
acgagcatcagcagcatacgacgtatcttttaggttttgtggtttgatgaagaagcggggaaagaagaccctgttgagcttgactctagtctggcacggtgaagagacatgagaggtgtagaataagaaaaaaaaaaaaaaaaaaaaaacattgcgcaatctgtctcttatacacatctgacgctgccgac

SEQ ID NO: 75 - DNA, 250 bp, Artificial Sequence
  Description of Artificial Sequence Synthetic polynucleotide
acgagcatcagcagcatacgaggggcatgttaggttttgtggtttgatgaatgagcgggtcaaccagctatcaccaggctcggtaggtttgtcgcctctacctataaatcttcccactattttgctacatagacgggtgtgctcttttagctgttcttaggtagctcgtctggtttcgggggtcttagctttggaaaaaaaaaaaaaaaaaacattgcgcaatctgtctcttatacacatctgacgctgc

(19) United States
(12) Patent Application Publication
(10) Pub. No.: US 2023/0029257 A1
(43) Pub. Date: Jan. 26, 2023

(54) COMPOSITIONS AND METHODS FOR LIGHT-DIRECTED BIOMOLECULAR BARCODING

(71) Applicant: PRESIDENT AND FELLOWS OF HARVARD COLLEGE, Cambridge, MA (US)

(72) Inventors: Jocelyn KISHI, Boston, MA (US); Ninning LIU, Boston, MA (US); Sinem SAKA, Allston, MA (US); Peng YIN, Brookline, MA (US); Kuanwei SHENG, Cambridge, MA (US)

(73) Assignee: PRESIDENT AND FELLOWS OF HARVARD COLLEGE, Cambridge, MA (US)

(21) Appl. No.: 17/783,750

(22) PCT Filed: Dec. 11, 2020
(86) PCT No.: PCT/US2020/064463

§ 371 Date: Jun. 9, 2022

Related U.S. Application Data
(60) Provisional application No. 62/947,237, filed on Dec. 12, 2019.

(51) Int. Cl. C12Q 1/6818; C12N 15/10

(52) U.S. Cl. CPC C12Q 1/6818; C12N 15/1065; C12Q 2600/158; C12Q 2600/16; C12Q 1/6841

(57) ABSTRACT

Provided herein are compositions, kits, and methods for nucleic acid barcoding. The barcode compositions provided herein can be used to linearly, combinatorially, or spatially barcode a plurality of targets in a sample. Also provided herein is a device for use in a barcoding method provided herein comprising a light source and a sample holder.

### CROSS-REFERENCE TO RELATED APPLICATION

This application claims benefit under 35 U.S.C. § 119(e) of U.S. Provisional Application No. 62/947,237 filed Dec. 12, 2019, the contents of which are incorporated herein by reference in their entirety.

### GOVERNMENT SUPPORT

This invention was made with government support under N00014-16-1-2410 and N00014-18-1-2549 awarded by the Department of Defense/Office of Naval Research; HL145600 and GM133052 awarded by the National Institutes of Health; and U.S. Pat. Nos. 1,317,291 and 1,729,397 awarded by the National Science Foundation. The government has certain rights in the invention.

### TECHNICAL FIELD

The present disclosure relates to compositions and methods for nucleic acid barcoding.

### BACKGROUND

To understand how cells function, differentiate, and respond to environmental factors, profiling molecular states of single cells in their native environment is necessary for basic research applications and biomedicine. Single-cell sequencing has revealed critical new understandings of biology by providing quantitative cell-level transcriptomics information. However, multiscale spatial information, both at the sub-cellular level and the level of cells positioned within a tissue, is lost in the process of dissociating cells for cell level sequencing.

### SUMMARY

Provided herein are compositions methods for light-directed barcoding followed by sequencing, that allows for programmable labeling of biomolecules across length scales (sub-cellular to large tissues) with barcode sequences that attach to nucleotide sequences in situ. The methods provided herein are high-throughput and have several advantages over previous methods for barcoding, for example, the ability to provide both sequence information with spatial information, improved signal to background noise ratio, multiplexing capability, improved detection speed, selectivity, scalability, and there is no need for pre-determined capture arrays or destruction of a sample.

In one aspect, provided herein is a composition, e.g., a barcode composition, comprising a first and second nucleic acid strands, where the first nucleic acid comprises in a 5′ to 3′ direction, an optional unique molecule identifier (UMI) sequence, a first targeting domain and a hybridization domain; and the second nucleic acid comprises in a 5′ to 3′ direction a barcode domain and a hybridization domain, wherein the hybridization domain of the first nucleic acid strand is substantially complementary to the hybridization domain of the second nucleic acid and at least one of the hybridization domain of the first nucleic acid strand and the hybridization domain of the second nucleic acid comprises a photo reactive element.

In another aspect, provided herein is a composition, e.g., a barcode composition, comprising a first and second nucleic acid strands, where the first nucleic acid comprises in a 5′ to 3′ direction an optional unique molecule identifier sequence, a first targeting domain and a hybridization domain; and the second nucleic acid comprises in a 5′ to 3′ direction a hybridization domain and a barcode domain, wherein the hybridization domain of the first nucleic acid strand is substantially complementary to the hybridization domain of the second nucleic acid and at least one of the hybridization domain of the first nucleic acid strand and the hybridization domain of the second nucleic acid comprises a photo reactive element.

In some embodiments, the second nucleic acid strand also comprises a unique molecule identifier sequence. For example, the unique molecule identifier sequence can be present 5′ to the barcode sequence, e.g., at the 5′-end. The second nucleic acid strand can also comprise a primer sequence. For example, embodiments, the second nucleic acid strand comprises a primer sequence. For Example, the second nucleic acid strand can comprise a primer sequence at a 5′-end to the barcode domain or the unique molecule identifier sequence. Generally, the primer sequence will be at or near the 5′-end of the second nucleic acid.

In some embodiments, a composition described herein further comprises a third nucleic acid strand, where the third nucleic strand comprises a barcode domain, wherein the barcode domain of the third nucleic acid is substantially complementary to the barcode domain of the second nucleic acid strand. In some embodiments, the third nucleic acid further comprises a unique molecule identifier sequence at the 5′-end of the barcode domain. The third nucleic acid can also comprise a primer sequence. For example, the third nucleic acid can also comprise a primer sequence at a 5′-end to the barcode domain or the unique molecule identifier sequence. Generally, the primer sequence will be at or near the 5′-end of the third nucleic acid

In still another aspect, provided herein is a composition, e.g., a barcode composition, comprising a first nucleic comprising in a 5′ to 3′ direction an optional unique molecule identifier sequence, a first targeting domain and a hybridization domain, and n additional nucleic acids, wherein n is an integer from 1 to 100, and wherein each additional nucleic acid comprises in 5′ to 3′ direction a first hybridization domain, a barcode domain; and a second hybridization domain, and wherein the first hybridization domain of nth nucleic acid is substantially complementary to the second hybridization domain of (n−1)th nucleic acid, wherein the first hybridization domain of n=1 nucleic acid is substantially complementary to the first hybridization domain of the first nucleic acid, and wherein at least one of the first or second hybridization domain of each nucleic acid comprises a photoreactive element, and wherein at least one of the hybridization domain of the first nucleic acid strand and the first hybridization domain of n=1 nucleic acid strand comprises a photoreactive element.

In some embodiments, the composition further comprises a first cap nucleic acid strand comprising in 5′ to 3′ direction a first cap hybridization domain, wherein the first cap hybridization domain is substantially complementary to the second hybridization domain of nth nucleic acid, and a second cap hybridization domain, and wherein at least one of the first cap hybridization domain and the second hybridization domain of the nth nucleic acid strand comprises a photoreactive element.

In some embodiments, the composition further comprises a first cap nucleic acid strand and a second cap nucleic acid strand, the second nucleic acid strand comprising in 5′ to 3′ direction a primer sequence domain; optionally, a unique molecular identifier sequence; and a hybridization domain, wherein the hybridization domain is substantially complementary to the second cap hybridization domain of the first cap nucleic acid, and wherein at least one of the second hybridization domain of the first cap nucleic acid strand and the hybridization domain of the second cap nucleic acid comprises a photoreactive element.

Nucleic acid strands of the compositions can comprise additional elements or domains. For example, the first nucleic acid can further comprise a primer sequence. The primer sequence can be present at a 5′-end to the targeting domain or the unique molecule identifier sequence. Generally, the primer sequence will be at or near the 5′-end of the first nucleic acid strand.

Also provided herein is a kit comprising a composition described herein. For example, a kit comprising the nucleic acid strands, and optionally additional elements or devices described herein.

The compositions and kits disclosed herein are useful for detecting and/or barcoding targets. The compositions and kits disclosed herein can be used for barcoding biomolecules in vitro, in vivo, in situ, or in toto. Accordingly, also provided herein are methods for barcoding or detecting target nucleic acids. In one aspect, provided herein is a method for detecting a target mRNA. Generally, the method comprises: (i) hybridizing a target mRNA (a first nucleic acid) with a second nucleic acid, and wherein the mRNA comprises a hybridization domain comprising a polyA sequence, and the second nucleic acid comprises in a 5′ to 3′ direction a hybridization domain and a first barcode domain, wherein the hybridization domain of the second nucleic acid is substantially complementary to the hybridization domain of the first nucleic acid, and at least one of the hybridization domains comprises a photoreactive element; and (ii) photocrosslinking the mRNA with the second nucleic acid thereby forming a probe-primer complex; (iii) synthesizing a record nucleic acid from the probe-primer complex; and (iv) detecting the record nucleic acid.

In another aspect, provided herein is a method for detecting a target nucleic. Generally, the method comprises: (i) hybridizing a target nucleic acid with a first nucleic acid and hybridizing a second nucleic acid with the first nucleic acid, wherein the first nucleic acid comprises in a 5′ to 3′ direction an optional unique molecule identifier (UMI) sequence, a targeting domain substantially complementary to a nucleic acid of the target element; and a hybridization domain, wherein the second nucleic acid comprises in a 5′ to 3′ direction a hybridization domain and a barcode domain, and wherein the hybridization domain of the second strand is substantially complementary to the hybridization domain of the first strand, and at least one of the hybridization domains comprises a photoreactive element; (ii) photocrosslinking the first nucleic acid with the second nucleic acid thereby forming a probe-primer complex; (iii) optionally, denaturing the probe-primer complex from the target nucleic acid; (iv) synthesizing a record nucleic acid from the probe-primer complex; and (v) detecting the record nucleic acid.

In still another aspect, provided herein is a method for detecting a target mRNA. The method comprises: (i) hybridizing a target mRNA (a first nucleic acid) with a second nucleic acid, wherein the mRNA comprises a hybridization domain comprising a polyA sequence, and wherein the second nucleic acid comprises in a 5′ to 3′ direction a hybridization domain, and a barcode domain, and wherein the hybridization domain of the second strand is substantially complementary to the hybridization domain of the mRNA and comprises a photoreactive element; (ii) photocrosslinking the mRNA with the second nucleic acid thereby forming a first complex; (iii) hybridizing a third nucleic acid to the second nucleic in the first complex thereby forming a probe-primer complex, wherein the third nucleic acid comprises a barcode domain substantially complementary to the first barcode domain of the second nucleic acid; (iv) synthesizing a record nucleic acid from the probe-primer complex; and (v) detecting the record nucleic acid.

Also provided herein is a method for detecting a target nucleic acid. The method comprises: (i) hybridizing a target nucleic acid with a first nucleic acid and hybridizing a second nucleic acid to the first nucleic acid, wherein the first nucleic acid comprises in a 5′ to 3′ direction an optional unique molecule identifier sequence, a targeting domain, and a hybridization domain, wherein the targeting domain is substantially complementary to the target nucleic acid, wherein the second nucleic acid comprises in a 5′ to 3′ direction a hybridization domain and a barcode domain, and wherein the second hybridization domain is substantially complementary to the first hybridization domain of the first nucleic acid and at least one of the hybridization domains comprises a photoreactive element; (ii) photocrosslinking the first nucleic acid with the second nucleic acid thereby forming a first complex; (iii) optionally, denaturing the first complex from the target nucleic acid; (iv) hybridizing a third nucleic acid to the second nucleic acid in the first complex thereby forming a probe-primer complex, wherein the third nucleic acid comprises a barcode domain substantially complementary to the barcode domain of the second nucleic acid; (v) synthesizing a record nucleic acid from the probe-primer complex; and (vi) detecting the record nucleic acid.

In yet another aspect, provided herein is a method for detecting a target nucleic acid. Generally, the method comprises preparing a concatemer. For example, the method comprises: (i) hybridizing a target nucleic acid with a first nucleic acid, wherein the first nucleic acid comprises in a 5′ to 3′ direction an optional unique identifier sequence, a targeting domain, and a hybridization domain, wherein the first targeting domain is substantially complementary to the target nucleic acid; (ii) preparing a concatemer by hybridizing, e.g., in a stepwise manner, n additional nucleic acids and photocrosslinking the additional nucleic acids with the first strand, wherein n is an integer from 1 to 100, and wherein each additional nucleic acid comprises in 5′ to 3′ direction a first hybridization domain, a barcode domain, and a second hybridization domain, wherein the first hybridization domain of nth nucleic acid is substantially complementary to the second hybridization domain of (n−1)th nucleic acid, wherein the first hybridization domain of n=1 nucleic acid is substantially complementary to the hybridization domain of the first nucleic acid, and wherein at least one of the first or second hybridization domain of each nucleic acid comprises a photoreactive element and at least one of the first hybridization domain of the n=1 nucleic acid and the hybridization domain of the first nucleic acid comprises a photoreactive element; (iii) hybridizing a first cap nucleic acid strand with the concatemer thereby forming a capped concatemer, wherein the first cap nucleic acid comprises a first cap hybridization domain, and a second cap hybridization domain, wherein the first cap hybridization domain is substantially complementary to the second hybridization domain of nth nucleic acid; (iv) hybridizing a second cap nucleic acid strand to the capped concatemer, thereby forming a concatemer-primer complex, wherein the second cap nucleic acid strand comprises in 5′ to 3′ direction a primer sequence domain, an optional unique molecular identifier sequence, and a hybridization domain, wherein the hybridization domain of the second cap nucleic acid is substantially complementary to the second cap hybridization domain of the first cap nucleic acid, and wherein at least one of the cap hybridization domain of the second cap nucleic acid and the second hybridization domain of the first cap nucleic acid comprises a photoreactive element; (v) detecting the concatemer-primer complex or synthesizing a record nucleic acid from the concatemer-primer complex and detecting the record nucleic acid.

Exemplary methods for detecting the record strand include, but are not limited to sequencing the record nucleic acid, light microscopy, high throughput scanner, confocal microscopy, light sheet microscopy, electron microscopy, atomic force microscopy, and/or the unaided eye.

In some embodiments, the record strand can be amplified prior to detection, e.g., sequencing. If desired, a photocrosslink linking two nucleic acid strands can be cleaved, uncrosslinked, removed, or reversed prior to amplifying and/or sequencing the record strand.

In another aspect, provided herein is a method for linearly, combinatorially or spatially barcoding a plurality of targets in a sample. Generally, the method comprises hybridizing a target nucleic acid strand in each member the plurality of targets with a first nucleic acid strand, followed by preparing a concatemer by hybridizing in a stepwise manner one or more additional nucleic acid strand and photocrosslinking the additional nucleic acid strands with the first complex, then detecting the concatemer and/or synthesizing a record nucleic acid from the concatemer and detecting the record nucleic acid.

The target nucleic acid strand can be comprised within another nucleic acid molecule, or the target nucleic acid strand is conjugated with a member of the plurality of targets, or the target nucleic acid strand is expressed by a cell, or the target nucleic acid strand is presented on a target or cell directly or indirectly via chemical crosslinking, genetic encoding, viral transduction, transfection, conjugation, cell fusion, cellular uptake, hybridization, DNA binding proteins or a target binding agent/ligand.

In some embodiments, the first nucleic acid strand comprises in a 5′ to 3′ direction: 1. optionally, a unique molecule identifier (UMI) sequence; 2. a first targeting domain, wherein the first targeting domain is substantially complementary to the target nucleic acid; and 3. a first hybridization domain. In some embodiments, the target nucleic acid strand is different in each member the plurality of targets. In some embodiments, the photocrosslinking step comprises selecting predetermined regions of the sample and exposing the predetermined regions to light after hybridizing each additional nucleic acid strand, thereby cross-linking the complementary hybridization domains, and removing any non-crosslinked additional nucleic acid strands after exposure to light and prior to hybridization a next additional nucleic acid strand.

In some embodiments, each additional nucleic acid strand comprises in 5′ to 3′ direction: i. a first hybridization domain; ii. a barcode domain; and iii. a second hybridization domain. In some embodiments, the first hybridization domain of nth additional nucleic acid strand is substantially complementary to the second hybridization domain of (n−1)th additional nucleic acid strand. In some embodiments, the first hybridization domain of the first additional nucleic acid strand is substantially complementary to the first hybridization domain of the first nucleic acid strand. In some embodiments, at least one of the first or second hybridization domain of each nucleic acid strand comprises a photoreactive element.

In yet another aspect, provided herein is a use of a method provided herein for screening a library of candidates for treatment. In some embodiments, the use comprises identifying one or more phenotypic markers by imaging and barcoding predefined regions by a method provided herein.

In another aspect, provided herein is a use of a method provided herein for identifying for screening of candidates, identification of drug targets, identification of biomarkers, profiling, characterization of phenotypic to genotypic cell state, generation of new disease models, characterization of cells and disease models, characterization of differentiation status and cell state, tissue mapping, multi-dimensional analysis, high content screening, machine-learning based clustering or classification, cell therapy development, CAR-T therapy development, antibody screening, personalized medicine, cell enrichment, and any combinations thereof.

In another aspect, provided herein is a device for use in a method provided herein. In some embodiments, the device comprises a light source and a sample holder.

### BRIEF DESCRIPTION OF THE DRAWINGS

FIG. 1A-1C shows dual light-directed barcoding (Strategy 1). FIG. 1A shows probe sequences are bound to targets of interest and later barcode-containing primers. If illuminated with the right wavelength of UV light, the primers become covalently linked (crosslinked) to probe sequences, and a polymerase is used to copy a full record strand before crosslinking is reversed with a different light wavelength. Record amplicons may first be PCR amplified before being submitted for sequencing. FIG. 1B shows probe sequences can bind to any entity labeled with a nucleic acid in addition to genomic/transcriptomic targets in situ, such as a DNA-conjugated antibody that is bound to a target protein. FIG. 1C shows a non-targeted approach can also be used for barcoding. For example, the polyA tail of mRNA transcripts can be bound to barcode primers, which can then be crosslinked as previously described. Reverse transcription is used to copy part or all of the mRNA transcript sequence before subsequent preparation steps and sequencing.

FIG. 2A-2D shows light-directed barcoding with barcoded bridge sequences (Strategy 2). FIG. 2A shows probe sequences are bound to targets of interest and later barcode-containing bridge strands. If illuminated with the appropriate wavelength of UV light, the bridges become covalently linked (crosslinked) to probe sequences, and probe-bridge complexes can be denatured before a corresponding primer is hybridized to the barcode sequence. A polymerase is used to copy a full record strand, which can then be PCR amplified before sequencing. If a strand displacing polymerase is used, the polymerization reaction can also happen when the probe is still bound to a target (part (FIG. 2B)). FIG. 2C shows a non-targeted approach can also be used for barcoding. For example, the polyA tail of mRNA transcripts may be bound to barcode bridges containing several T bases. FIG. 2D shows that these barcode bridges can then be crosslinked and prepared for sequencing (with reverse transcription, etc.) as previously described. Sequencing is then used to recover transcript plus barcode information.

FIG. 3A-3C shows light-directed barcoding with concatemer assembly (Strategy 3). FIG. 3A shows probe sequences are bound to targets of interest and later barcode strands. If illuminated with the right wavelength of UV light, barcodes become covalently linked (crosslinked) to probe sequences. Concatemers are formed through iterative barcode hybridization and crosslinking reactions. FIG. 3B shows that a strand displacing polymerase is used to copy a full record strand through a cross-junction synthesis reaction, which can then be PCR amplified before sequencing. Sequences reveal combined barcode sequence and target sequence information. The concatemer assembly may also first be denatured from the sample/surface before priming and cross-junction synthesis (part (FIG. 3C)).

FIG. 4A-4D shows light-directed barcoding. FIG. 4A shows the basic sequence-specific crosslinking reaction involves two complementary or largely complementary sequences, with one containing a CNVK modification, binding to each other. Exposure to UV light causes a covalent linking of the strands (crosslinking). FIG. 4B shows that by confining illumination to a specific region or set of regions, crosslinking can also be confined to these regions (using Strategy 1 chemistry as previously described). For example, CNVK-containing probe sequences are bound, but only some regions are crosslinked, then after washing away all non-crosslinked strands results in probes bound only in the illuminated region(s). FIG. 4C shows iterative rounds of hybridization, spatially patterned crosslinking, and washing using barcode primers with different barcode sequences (e.g. B1 through Bn) can be used to label distinct regions. After sequencing, which can happen with all records being synthesized simultaneously and pooled during sequencing, the combined barcode sequence and target/transcript information is recovered. The iterative spatially patterned crosslinking can also be done similarly for the second barcoding chemistry described previously (Strategy 2), but with barcode bridge strands bound in different rounds rather than different barcode primers (part (FIG. 4D)).

FIG. 5A-5C shows light-directed combinatorial barcoding. FIG. 5A shows combinatorial light-directed barcode assembly is achieved via iterative rounds of hybridization, spatially patterned crosslinking, and washing of barcode strands with different barcode sequences (e.g. sequences 0 and 1). FIG. 5B shows each individual region can receive a unique assembly order (e.g. 1010010 or 0011101 in the example shown), or multiple regions may receive the same assembly sequence if desired. FIG. 5C shows the order of assembled barcode sequences plus the original probe sequence information is synthesized in a record strand through a cross-junction synthesis reaction. PCR amplification may be performed before records are sequenced.

FIG. 6A-6F demonstrates experimental validation of spatially patterned crosslinking. FIG. 6A shows CNVK (gray filled circle) modified barcoding strands are used in combination with a spatial light mask to direct crosslinking of barcodes towards RNA targets in a selection of cells. Barcoding strands contain both a barcode sequence (blue and purple) and a Cy3b fluorophore (green star). Iterative light-directed barcode construction can proceed through successive washes and UV crosslinking events FIG. 6B shows a final crosslinking step shown which will deliver and crosslink a strand that carries a primer binding site (orange) for a Cy5 labeled primer strand (orange strand with magenta star). Whole field crosslinking was performed for this step. FIG. 6C shows DAPI (blue channel) labeled EY.T4 cells. No crosslinking. FIG. 6D shows a spatial mask was applied to crosslink the ribosomal RNA of the cells with a Cy3b (green channel) labeled barcoding strand. Green channel illustrates successful crosslinking in a cross-rectangle pattern after a formamide wash. FIG. 6E shows a closer field of view of panel (d) at the ‘intersection’ point between the two rectangles. FIG. 6F shows imaging in DAPI (blue), Cy3b (green), and Cy5 (magenta) channel after the final primer capping set show in in panel (FIG. 6B). Cy5 labeled strands are expected to crosslink to all cells due to whole field UV crosslinking. Cells containing both barcoded strands and primer strands are overlaid in both green and magenta channel and are expected to appear white in the channel overlay. Note, the magenta channel contrast was scaled to match the barcoded cells which are expected to have 3× higher Cy3b fluorophores compared to Cy5.

FIG. 7A-7C shows iterative assembly of concatemers up to 3 junctions. FIG. 7A shows schematic for iterative junction assembly with Cy3b-labeled barcode strands and a Cy5-labeled primer. FIG. 7B shows a representative schematic for cross-junction synthesis of one- and three junction assemblies followed by PCR amplification of records. FIG. 7C shows PAGE denaturing gel showing PCR products for two experiments and no probe control.

FIG. 8A-8C shows experimental validation of cell-level spatial labeling. FIG. 8A shows a mixture of cells displaying different phenotypic markers. GFP transfected cells (green circle) are selected for crosslinking with CNVK strands (gray filled circle) carrying a reporter fluorophore (orange star). FIG. 8B shows an overlay of brightfield and green channel images showing a mixture of GFP transfected and none transfected cells. Multiple regions of interest (yellow, blue, green, red outlines) selected for cross linking are drawn around the cells displaying GFP signal. FIG. 8C shows fluorescent image of cells after crosslinking. Nuclei stain (blue), GFP (green), and the fluorescent CNVK strand (yellow) are overlaid.

FIG. 9A-9D shows sequencing results. Utilizing a variant of Strategy 2, with UMIs on both ends of the amplicon, three distinct spatially separated regions were serially barcoded using patterned illumination on fixed HeLa cells. FIG. 9A demonstrates that 6 distinct probe sequences (two targeting ribosomal RNA and four targeting the Xist RNA) were bound to their target RNA sequences with FISH. This was followed by iterative barcoding, binding of barcode-containing primers, synthesis, and amplification of records. Amplicons were prepared for Next Generation Sequencing (HiSeq) using a Collibri sequencing prep kit. FIG. 9B-9C show reads of the anticipated format were recovered with high percentage following alignment. FIG. 9D shows read distributions for a large subset of the data are shown for each probe-region pair.

FIG. 10 demonstrates targeted and non-targeted approaches of barcoding. Any type of nucleic acid may be barcoded. These nucleic acids are typically associated with, bound to, or hybridized to biomolecules localized in situ. Specific biomolecules can be targeted through a targeted or affinity-based approach, such as FISH for DNA/RNA targets, IF for protein targets (e.g. via a nucleic acid-conjugated antibody or nanobody), or any other affinity-based reagent capable of being conjugated or otherwise associated with a nucleic acid. A non-targeted may instead be utilized, whereby nucleic acids are localized or generated in a non-targeted fashion. For example cDNA copies produced from reverse transcription of RNA, or pre-existing RNA or DNA or modified backbone sequences or other reaction products in situ generated by the action of polymerases, ligases, restriction enzymes, nucleases, telomerases, terminal transferases, recombinases or transposases such as those of proximity ligation assay, primer exchange reaction, autocyclic proximity recording, or tagmentation, can be barcoded.

FIG. 11A-11B shows an assembly of barcodes for a cell or other region of interest. (FIG. 11A) Iterative formation of a concatemer upon nucleic acids localized in situ (e.g. cDNA sequences) results in the formation of a specific barcode for reads from that cell (e.g. m-g-o-m-y-r-c). Orientation shown for 3′ barcoding of cDNA, although 5′ barcoding may also be performed (see e.g. FIG. 18 and FIG. 19). (FIG. 11B) Cross junction synthesis and PCR are used to prepare records for sequencing.

FIG. 12 shows application of the methods and compositions provided herein.

FIG. 13 shows a workflow for dissociative split-pool barcoding. Iterative splitting of cells or otherwise associated biomolecules (e.g. hydrogel pieces) into tubes, barcoding of nucleic acids e.g. with the light-directed concatemer formation depicted elsewhere, and then re-pooling enables unique barcode sequences to be associated with each separate cell/component. Split-pool strategies have previously been used for single-cell barcoding through multiple expensive enzymatic ligation steps but using the concatemer-based barcoding strategy dramatically lowers costs as each barcoding step can be performed without the need for expensive enzymes or other reagents. Sequences can be extracted similarly to when they're on a surface: with cross-junction synthesis and PCR of records.

FIG. 14A-14C shows an embodiment of the spatial barcoding. (FIG. 14A) Barcodes are crosslinked typically through the use of a CNVK modification, and crosslinking is activated with UV light. (FIG. 14B) By spatially addressing UV light illumination profiles, barcodes may be crosslinked to dock sequences only in desired positions, and after a stringent wash step (e.g. a formamide-containing buffer) all non-crosslinked barcode strands can be washed away. (FIG. 14C) Iterative steps of binding, crosslinking specific regions, and stringent washing enables the iterative construction of barcodes associated with those specific regions.

FIG. 15A demonstrates linear barcoding of N regions (e.g. N distinct cells) is performed such that a single of N barcodes is assigned to each position or (or positions) of interest. Sequencing results may then be extracted together in bulk, and reads may be mapped back to their original corresponding positions based on the barcode sequences in the reads. FIG. 15B demonstrates a method of combinatorial barcoding, a concatenated barcode is iteratively constructed, such that each region (e.g. cell) that for which reads should be attributed to receives a unique barcode (see e.g. FIG. 18). For example, for N rounds of M barcodes, MAN unique barcodes could feasibly be assigned.

FIG. 16 shows an embodiment of the workflow for combined imaging and RNA sequencing data for a sample. In general, extra imaging steps and other assays may be added before or after barcoding, and the A-tailing step may optionally occur before or after barcoding. A different tailing (e.g. a T-tailing, C-tailing, G-tailing, or any other type of tailing with terminal transferase or other enzyme may be utilized) may instead be utilized. For targeted approaches, the workflow is very similar, except that probes may already contain the 5′ and 3′ tails, so both RT and A-tailing steps can be skipped. Any domain (e.g., 1-letter, 2-letter, 3-letter, or 4-letter) may be utilized for the 3′ tail sequence.

FIG. 17 shows experimental validation of UV power and illumination conditions. A set of experiments to optimize the UV power and illumination conditions for barcoding FISH probes bound to rRNA transcripts in HeLa cells. A checkerboard pattern was rastered across a well with each separate region testing a different UV power and illumination time condition.

FIG. 18 shows a strand diagram of 5′ light-directed barcoding strategy with UMI on cross-junction synthesis primer. A primer with an overhanging 5′ domain (e.g. with random N bases on the end) is localized to RNA's (e.g. mRNA, non-coding RNAs) and creates cDNA sequences. The cDNA sequences may then be appended with bases on the 3′ end, such as with a polyA tail with the use of terminal transferase and dATP. Subsequently, combinatorial barcodes are assembled iteratively directly onto the 5′ overhang of the cDNA or other in situ localized sequence, through binding, UV crosslinking, and wash steps. (The A tailing step may be included before or after barcoding). Optionally, RNaseH displacement of barcodes from RNA may be performed before or simultaneously with cross junction synthesis. After cross-junction synthesis, full records are formed via PCR amplification.

FIG. 19 shows a strand diagram of 5′ light-directed barcoding strategy with UMI on barcode capping strand. A primer with an overhanging 5′ domain (e.g. with random N bases on the end) is localized to RNA's (e.g. mRNA, non-coding RNAs) and creates cDNA sequences. The cDNA sequences may then be appended with bases on the 3′ end, such as with a polyA tail with the use of terminal transferase and dATP. Subsequently, combinatorial barcodes are assembled iteratively directly onto the 5′ overhang of the cDNA or other in situ localized sequence, through binding, UV crosslinking, and wash steps. Optionally, RNaseH displacement of barcodes from RNA may be performed before or simultaneously with cross junction synthesis. After cross-junction synthesis, full records are formed via PCR amplification.

FIG. 20 shows experimental validation of primer sets for cDNA library generation. (top) A table of primers and concentrations used for reverse transcription (RT). Well labels (A1-B4) match the orientation of images shown in bottom. Well B1-B4 have a combination of primers as well as a non-reverse transcribed negative control. (bottom) Images of the localization of the cDNA library after reverse transcription using a Cy5 labeled primer. A Cy3 CNVK barcode was then added and crosslinked in a checkerboard pattern using a DMD and a 10× objective and imaged in Cy3.

FIG. 21 shows sequencing results for different RT primers. In situ reverse transcription in fixed HeLa cells was performed with different primers containing 5′ barcoding domains along with NNNN (7N's, experiment A1), NNNNNGGG (5N's and 3G's, experiment A2), or CCC (5N's and 3C's, experiment A3) on the 3′ end. After barcoding, cross-junction synthesis, and PCR according to the strategy depicted in FIG. 18, PCR amplicons were purified with Ampure XP beads and sent for sequencing (250 bp paired end). Examples of several expected read results are shown for each of these primers, and the highlighted cDNA sequences (blue) map to known Homo sapiens sequences as expected. These data verify the success of the general strategy and that each primer may be used to successfully produce transcriptomic records.

FIG. 22A shows the sequence structure for barcoding a 5′ sequence (e.g. a 5′ tail on cDNA, FISH probe, etc.). A concatemer formed with a Reverse (Rev) primer capping strand, zero or more barcode strands, and a cDNA, FISH, or other probe sequence with a polyA tail can be effectively copied with a cross junction synthesis primer containing a Forward (For) primer and polyT 3′ end to form a PCR amplifiable record that can be sequenced. In this case, two different orientations of barcode sequences (W/X domains, and Y/Z domains) are utilized, though more distinct barcode sequences may be utilized as well. Strands may be purified or unpurified and may contain extra bases on the 3′ or 5′ ends (e.g. T linkers, fluorophores, modifications to prevent extension or degradation). FIG. 22B shows an embodiment of a binding domain barcode sequences used for the demonstrations in the next several figures are shown, colored according to their domains. An arbitrary number of barcode strands with different (Barcode) domain sequences may be utilized for barcoding. FIG. 22C shows complete sequence information for the experiments reported in the all subsequent figures are shown. PCR Primer sequences are based on the Smart Seq3 protocol. All other sequences and particularly those for barcoding have been specifically designed and experimentally for this barcoding application, after modeling and extensive testing of dozens of cross junction synthesis reactions. See also, Tables 1-3 in the working examples.

FIG. 23A-23E shows validation of iterative barcode assembly on a streptavidin-coated surface (glass slide). FIG. 23A shows a schematic of iterative barcode assembly of fluorescently labeled DNA barcode strands, followed by cross-junction synthesis and PCR. FIG. 23B shows schematics of concatenated barcodes with 2 to 7 junctions, containing 1 to 6 barcodes, respectively. FIG. 23C shows distribution of DNA barcode lengths expected in distinct wells (top). Top left well in an 8-well chamber contains DNA barcodes of length 6 and will display the highest amount of fluorescent signal. Followed by 5 and 4 etc. Scan of the 8-well chamber in the Cy3 Fluorescent channel (bottom). FIG. 23D shows complete sequence design for the 7-junction concatemer and amplicons based on sequences presented in FIG. 22A-22C. FIG. 23E shows that after extraction, PCR, and purification with a MinElute PCR Purification column, amplicons from the top left well (6-junction) were sequenced (250 bp paired end sequencing). Example sequencing results are shown, both for full length (6-barcode containing reads) as well as truncated reads (e.g. containing 2 or 4 barcodes). Truncated reads are expected in addition to full length reads due to some inefficiencies in the concatemer formation step.

FIG. 24 shows sequencing results for several different fixation, permeabilization, RT, and barcoding conditions following the strategy depicted in FIG. 19. (top) Several sequences that were acquired for each of several fixation/permeabilization conditions (experiments B1 through B8) and match the expected sequence format after two rounds of barcoding are shown. These sequences show the expected barcode sequences in each case and examples of different UMI's, and sequence lengths, that occur. (bottom) While keeping the fixation and permeabilization constant, several variations to the RT step were tested along with some controls. For each of experiments C1 through C4, one barcode was first introduced but not crosslinked prior to stringent washing (Exchange 1), and then a second barcode was introduced that was crosslinked with UV and should have shown up in the sequencing reads (Exchange 2). As expected, in all conditions except the control that contained RNase A during RT, the correct barcode that was crosslinked shows up in the majority of reads (>1,500 of 2,000 reads examined), and the incorrect (non-crosslinked Exchange 1 barcode) barcode showed up extremely rarely (as low as 0 in 2,000 reads). In all of the conditions (experiments B2 through B8, C1 through C4) except the no reverse transcriptase (RT) control (experiment B1), the highlighted cDNA sequences (blue) map to known Homo sapiens sequences. Exceptions: some conditions having A-tailing take place after barcoding, as indicated in the figure, and all conditions having the RNaseH treatment combined with the cross-junction synthesis incubation.

FIG. 25A-25D demonstrates imaging and gel results for experiments B1 through B8 and C1 through C4. FIG. 25A shows imaging results for experiments B1 through B8 show distinct fluorescence morphologies after reverse transcription (RT) with a fluorophore (Alexa 488)-labeled RT primer. As expected, after displacement, the fluorescence signal from localized primers goes significantly down, indicating they have been successfully displaced during the combined RNaseH and cross junction synthesis steps. FIG. 25B shows tor the control condition containing RNase A and no RNaseOUT during RT, signal was much higher, and lower contrast visualization revealed strong suspected nucleolar signal. FIG. 25C shows imaging results for experiments C1, C3, and C4 are also shown. FIG. 25D shows gel results for all conditions show the lengths of records produced after PCR amplification (1% Agarose E-gel with Sybr Gold). For cases containing reverse transcription and no RNase A, the typical lengths recovered range between about 150 bp and 1300 bp.

FIG. 26 shows transcriptomic mapping results. Transcriptomic mapping was performed with the STAR aligner on sequencing results. (left) An example output log file is shown on the left for mapping results for 1,024 transcripts identified with the expected sequence format for experiment B7. 40.5% of the reads mapped uniquely, whereas 49% mapped to multiple loci and 9.5% were too short to map. (right) Gene mapping results were sorted by frequency of mapped transcripts and the top of the list is depicted. The most common uniquely mapped genes correspond to mitochondrial rRNA.

FIG. 27 shows automated barcode assignment and iterative barcoding on a surface. An example workflow whereby a list of barcodes (BC1, BC2, BC3 etc. . . . ) can be converted into a series of photomasks (middle panel) with each region of interest (white squares, middle panel) assigned a unique barcode. An image was taken after a series of 6 barcoding steps with fluorescent DNA strands to uniquely tag and barcode an array of 112 regions of interest (right panel).

FIG. 28A-28G shows automated barcoding of biomolecular samples. FIG. 28A shows a workflow whereby a collection of cells can be detected with a computer algorithm and selectively targeted for barcode delivery, resulting in each cell with a unique barcode assignment.

FIG. 28B shows an image of cells with a fluorescent DNA primer targeting RNA. FIG. 28E shows an image of cells after 6 rounds of barcoding with a fluorescent DNA barcode (green) using the masks from panel (FIG. 28C, 28F). FIG. 28C and FIG. 28F show an overlay of the detected cellular masks (white outlines). FIG. 28D and FIG. 28G show an enlarged image of the outlined square from (FIG. 28C) and (FIG. 28F) respectively

### DETAILED DESCRIPTION

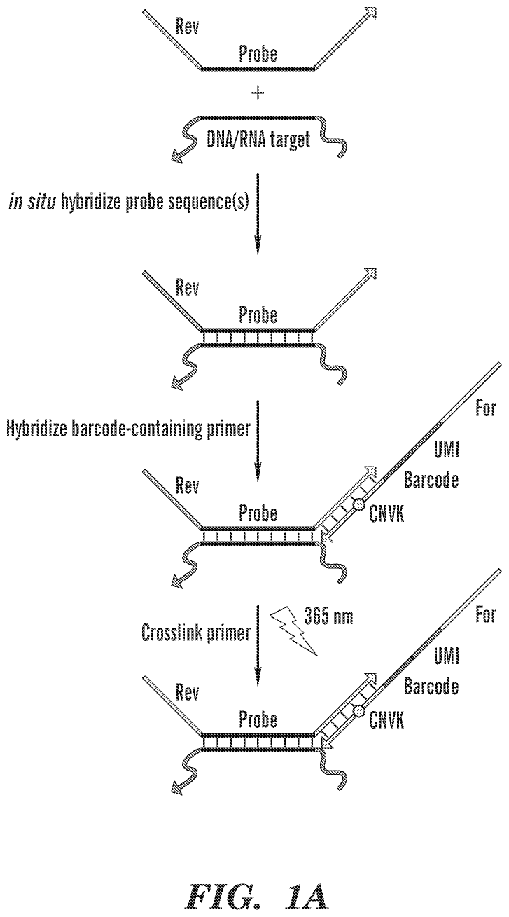

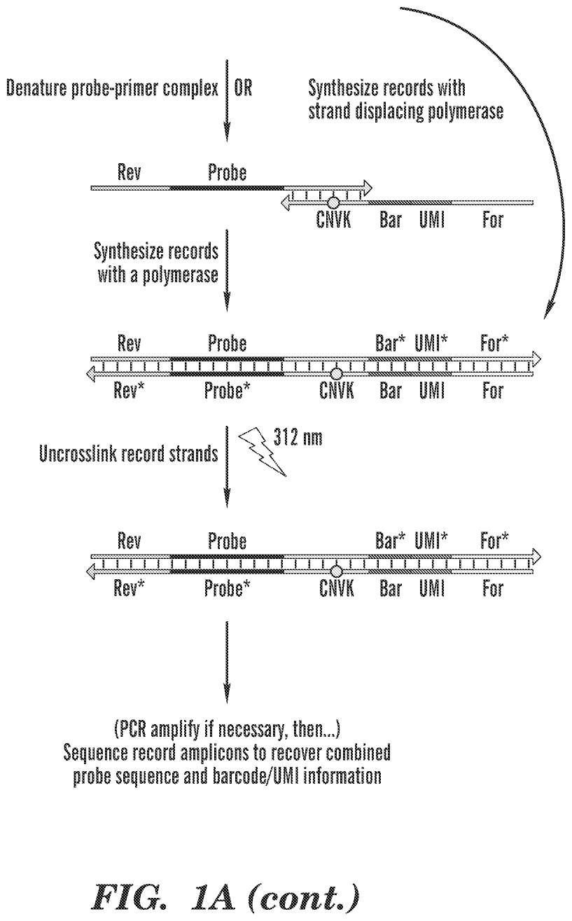

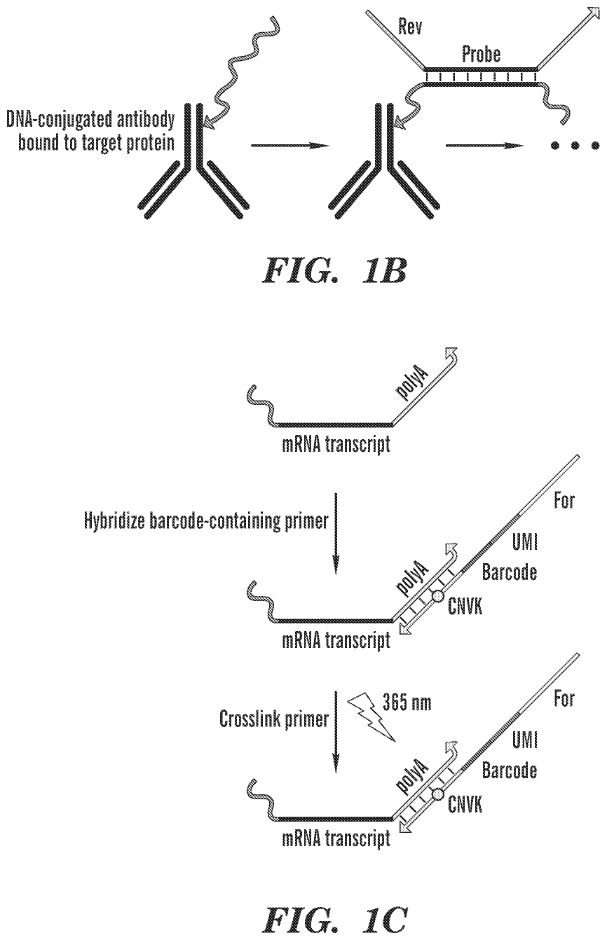

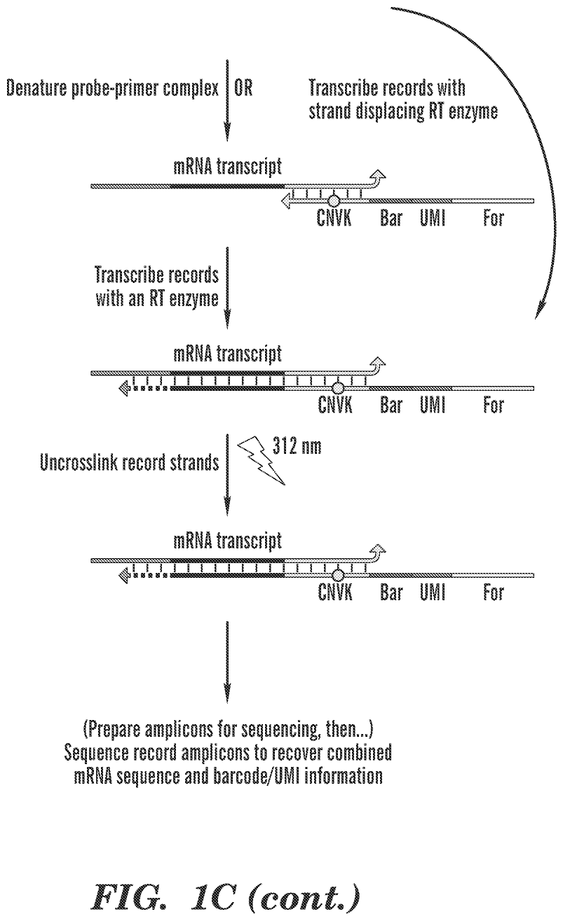

The fundamental strategy for nucleic acid barcoding provided herein is depicted in FIGS. 1A-9D.

Generally, the methods provided herein are based in part, on the discovery of methods and compositions that allow for high-throughput detection of a target nucleic acid and the production of sequence and spatial information. The methods and compositions provided herein are useful in many applications, such diagnostics, pathology, and basic research.

In particular, the compositions and methods provided herein can be useful in spatial mapping, detecting biomolecule localization, identifying various cell types in a tissue, molecular coding, data storage, tissue engineering, communication, and biosensing. The approaches provided herein can be used to create patterned and barcoded surfaces for oligonucleotide arrays. For example, the methods and compositions provided herein can be used for higher levels of patterning, masking, and capturing nucleic acid targets (e.g., biomarkers of interest).

As another example, the targeted approach provided in the working examples (e.g., Strategy 1), can also be used to bind other nucleic acids immobilized in a sample or on a surface, such as DNA-conjugated antibodies bound to protein targets of interest (see FIG. 1B). In general, any entity (such as nucleic acids, proteins, peptides, lipids, sugar groups, small molecules, nanoparticles, beads, glass surfaces) that can be labeled with or crosslinked to a strand of interest can be patterned, barcoded and recorded using the methods provided herein.

In some embodiments, the barcode composition comprises:
a. a first nucleic acid comprising in a 5′ to 3′ direction: (i) optionally, a unique molecule identifier (UMI) sequence; (ii) a first targeting domain; and (iii) a first hybridization domain, and
b. a second nucleic acid comprising in a 5′ to 3′ direction: (i) a barcode domain; and (ii) a second hybridization domain, wherein the second hybridization domain is substantially complementary to the first hybridization domain of the first nucleic acid, and

wherein at least one of the first or second hybridization domain comprises a photoreactive element.

In some embodiments, the barcode composition comprises:
a. a first nucleic acid comprising in a 5′ to 3′ direction: (i) optionally, a unique molecule identifier sequence; (ii) a first targeting domain; and (iii) a first hybridization domain; and
b. a second nucleic acid comprising in a 5′ to 3′ direction: (i) a second hybridization domain, wherein the second hybridization domain is substantially complementary to the first hybridization domain of the first nucleic acid; and (ii) a first barcode domain, and
wherein at least one of the first or second hybridization domain comprises a photoreactive element.

In some embodiments, the barcode composition comprises:
a. a first nucleic acid comprising in a 5′ to 3′ direction: (i) optionally, a unique molecule identifier sequence; (ii) a first targeting domain; and (iii) a first hybridization domain; and
b. a second nucleic acid comprising in a 5′ to 3′ direction: (i) a second hybridization domain, wherein the second hybridization domain is substantially complementary to the first hybridization domain of the first nucleic acid; and (ii) a first barcode domain; and (iii) a third hybridization domain, and
wherein at least one of the first or second hybridization domains comprises a photoreactive element, and the third hybridization domains optionally comprises a photoreactive element.

In some embodiments, the barcode composition further comprises n additional nucleic acids, wherein: n optionally is an integer from 1 to 100, and each additional nucleic acid comprises in 5′ to 3′ direction: (i) a first hybridization domain; (ii) a barcode domain; and (iii) a second hybridization domain, and wherein the first hybridization domain of nth nucleic acid is substantially complementary to the second hybridization domain of (n−1)th nucleic acid, wherein the first hybridization domain of n=1 nucleic acid is substantially complementary to the third hybridization domain, and wherein at least one of the first or the second hybridization domain of each nucleic acid comprises a photoreactive element.

In some embodiments, the barcode composition further comprises a first cap nucleic acid strand comprising in 5′ to 3′ direction: (i) a first cap hybridization domain, wherein the first cap hybridization domain is substantially complementary to the second hybridization domain of nth nucleic acid when n is 1 or more, or the cap hybridization domain is substantially complementary to the third hybridization domain when n is 0; and (ii) a second cap hybridization domain, wherein the first cap hybridization domain optionally comprises a photoreactive element.

In some embodiments, the barcode composition further comprises a first cap nucleic acid strand and a second cap nucleic acid strand, the second cap nucleic acid strand comprising in 5′ to 3′ direction: (i) a primer sequence domain; (ii) optionally, a unique molecular identifier (UMI) sequence; and (iii) a hybridization domain, wherein the hybridization domain is substantially complementary to the second cap hybridization domain of the first cap nucleic acid, and wherein at least one of the second cap hybridization domain and the hybridization domain of the second nucleic acid comprises a photoreactive element.

The nucleic acid strands of the compositions and methods described herein comprise one or more domains. Without limitation, each domain can independently comprise any desired nucleotide sequence or number of nucleotides. In other words, each domain can be independently of any length. Accordingly, each domain can be independently one nucleotide to thousands of nucleotides in length. For example, each domain can be independently 1 to 1000, 1 to 500, 1 to 250, 1 to 200, 1 to 150, 1 to 100, 1 to 75, 1 to 50, or 1 to 25 nucleotides in length. In some embodiments, each domain can be independently 1, 2, 3, 4, 5, 6, 7, 8, 9, 10, 11, 12, 13, 14, 15, 16, 17, 18, 19, 20, 21, 22, 23, 24, 25 or more nucleotides in length.

As described herein, hybridization domains of two nucleic strands can hybridize with each other to form a double-stranded structure. Without limitations, each duplex region can independently comprise any desired number of base-pairs. In other words, each duplex region can be independently of any length. Accordingly, each duplex region can be one base pair to tens of base pairs in length. In some embodiments, each duplex region can be independently 1 to 50, 1 to 45, 1 to 40, 1 to 35, 1 to 30, 1 to 25, 1 to 20 or 1 to 15 nucleotides or base pairs in length. For example, each duplex region can be independently 1, 2, 3, 4, 5, 6, 7, 8, 9, 10, 11, 12, 13, 14, 15, 16, 17, 18, 19, 20, 21, 22, 23, 24, 25 or more nucleotides or base pairs in length.

Each nucleic acid strand can be independently of any length. For example, each nucleic acid strand can be few nucleotides to thousands of nucleotides in length. For example, each nucleic acid strand can be independently 1 to 50, 1 to 75, 1 to 100, 1 to 150, 1 to 175, 1 to 200, 1 to 250, 1 to 300, 1 to 400, 1 to 500, 1 to 750, 1 to 1000 or more nucleotides in length.

Each domain can independently comprise any desired nucleotide sequence. Further, each domain can independently utilize a 1-letter, 2-letter, 3-letter or 4-letter code. As used herein, a “1-letter code” means the domain only comprises only one type of nucleobase, i.e., only one of adenine, thymine/uracil, guanine, and cytosine, or modified versions thereof. For example, a domain utilizing a 1-letter code comprises a stretch of nucleotides comprising the same nucleobase or a modified version of the nucleobase. For example, a domain can comprise a stretch of polyA, polyT, polyC or polyG. In some embodiments, the hybridization domain of the first nucleic acid utilizes a 1-letter code. For example, the hybridization domain of the first nucleic acid can comprise a poly(A) sequence.

A “2-letter code” means the domain only comprises two of the four nucleobases, i.e., only two of adenine, thymine/uracil, guanine, and cytosine, or modified versions thereof. For example, a 2-letter code can comprise or consist of nucleobases selected from the group consisting of adenine and thymine/uracil, adenine and guanine, adenine and cytosine, thymine/uracil and guanine, thymine/uracil and cytosine, and guanine and cytosine.

A “3-letter code” means the domain comprises only three of the four nucleobases, i.e., only three of adenine, thymine/uracil, guanine, and cytosine, or modified versions thereof. For example, a 3-letter code can comprise or consists of nucleobases selected from the group consisting of: adenine, thymine/uracil, and guanine; adenine, thymine/uracil, and cytosine; adenine, guanine, and cytosine; and thymine/uracil, guanine, and cytosine.

In some embodiments, at least one domain comprises same types of nucleobases. For example, a domain only comprises purine nucleobases or pyrimidine nucleobases.

The first nucleic acid strand can be an RNA molecule, e.g., an RNA transcript. In one example, the first nucleic acid is an mRNA. For example, the first nucleic strand is an mRNA and the hybridization domain comprises a polyA sequence.

As described herein, a nucleic acid strand comprises a unique molecule identifier sequence or domain. A unique molecule identifier sequence or domain can be synthesized by using a mix of nucleotides during base addition chemical synthesis to create libraries of random sequences (degenerate sequences). A unique molecule identifier sequence or domain can consist of several such random bases in tandem, with or without known nucleotide sequences intercalated. In some embodiments, a unique molecule identifier sequence or domain is excluded from primers and record sequences. In some embodiments, the unique molecule identifier sequence or domain of a nucleic acid is incorporated into one of the other domains of same nucleic acid.

As described herein, hybridization domains can comprise a photoreactive element. As used herein, the term “photoreactive element” refers to any element (e.g., nucleotide, protein, or antibody) that can permit hybridization to another nucleotide upon photoirradiation by a light source. In some embodiments, the photoreactive element is a photoreactive nucleotide. In some embodiments, the photoreactive nucleotide is a CNVK or CNVD crosslinking base. In some embodiments, the photoreactive element is psoralen.

In some embodiments of any of the aspects described herein, a nucleic acid strand can comprise a nucleic acid modification. For example, at least one of a targeting domain, a barcode domain, a hybridization domain, unique molecule identifier sequence and/or primer sequence domain can independently comprise a nucleic acid modification. Exemplary nucleic acid modifications include, but are not limited to, nucleobase modifications, sugar modifications, inter-sugar linkage modifications, conjugates (e.g., ligands), and any combinations thereof. Nucleic acid modifications also include unnatural, or degenerate nucleobases.

Exemplary modified nucleobases include, but are not limited to, inosine, xanthine, hypoxanthine, nubularine, isoguanisine, tubercidine, and substituted or modified analogs of adenine, guanine, cytosine and uracil, such as 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine,7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3 carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613.

In some embodiments, a modified nucleobase can be selected from the group consisting of: inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyl)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine,2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 5-ethynyl-2′-deoxyuridine, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil,4-(thio)pseudouracil,2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin−1-yl, 1-(aza)-2-(thio)-3-(aza)-phenoxazin−1-yl, 1,3-(diaza)-2-(oxo)-phenthiazin-1-yl, 1-(aza)-2-(thio)-3-(aza)-phenthiazin−1-yl, 7-substituted 1,3-(diaza)-2-(oxo)-phenoxazin-1-yl, 7-substituted 1-(aza)-2-(thio)-3-(aza)-phenoxazin−1-yl, 7-substituted 1,3-(diaza)-2-(oxo)-phenthiazin−1-yl, 7-substituted 1-(aza)-2-(thio)-3-(aza)-phenthiazin−1-yl, 7-(aminoalkylhydroxy)-1,3-(diaza)-2-(oxo)-phenoxazin−1-yl, 7-(aminoalkylhydroxy)-1-(aza)-2-(thio)-3-(aza)-phenoxazin−1-yl, 7-(aminoalkylhydroxy)-1,3-(diaza)-2-(oxo)-phenthiazin−1-yl, 7-(aminoalkylhydroxy)-1-(aza)-2-(thio)-3-(aza)-phenthiazin−1-yl, 7-(guanidiniumalkylhydroxy)-1,3-(diaza)-2-(oxo)-phenoxazin−1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin−1-yl, 7-(guanidiniumalkylhydroxy)-1-(aza)-2-(thio)-3-(aza)-phenthiazin−1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, and any O-alkylated or N-alkylated derivatives thereof.

Exemplary sugar modifications include, but are not limited to, 2′-Fluoro, 3′-Fluoro, 2′-OMe, 3′-OMe, 2′-deoxy modifications, and acyclic nucleotides, e.g., peptide nucleic acids (PNA), unlocked nucleic acids (UNA) or glycol nucleic acid (GNA).

In some embodiments, a nucleic acid modification can include replacement or modification of an inter-sugar linkage. Exemplary inter-sugar linkage modifications include, but are not limited to, phosphotriesters, methylphosphonates, phosphoramidate, phosphorothioates, methylenemethylimino, thiodiester, thionocarbamate, siloxane, N,N′-dimethylhydrazine (—CH2-N(CH3)-N(CH3)-), amide-3 (3′-CH₂—C(═O)—N(H)-5′) and amide-4 (3′-CH₂—N(H)—C(═O)-5′), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′-CH₂—NH—NH—C5′, 3′-NHP(O)(OCH₃)—O-5′ and 3′-NHP(O)(OCH₃)—O-5′.

In some embodiments, nucleic acid modifications can include peptide nucleic acids (PNA), bridged nucleic acids (BNA), morpholinos, locked nucleic acids (LNA), glycol nucleic acids (GNA), threose nucleic acids (TNA), or any other xeno nucleic acids (XNA) described in the art.

In some embodiments of the various aspects described herein, a nucleic acid can be independently modified on the 3′- and/or 5′-end. For example, a label, fluorophore, tag, or a cap can be added to the 3′ and/or 5′-end of a nucleic acid described herein.

In some embodiments of the various aspects described herein, a nucleic acid strands described herein can be modified with a linker or spacer, e.g., at an internal position, on the 3′- and/or 5′-end. Without wishing to be bound by a theory, the linker or spacer can be used for linking the nucleic acid strand with a moiety, such as a solid support or label. In some embodiments, the linker or spacer can be selected from the group consisting of photocleavable linkers, hydrolyzable linkers, redox cleavable linkers, phosphate-based cleavable linkers, acid cleavable linkers, ester-based cleavable linkers, peptide-based cleavable linkers, and any combinations thereof. In some embodiments, the cleavable linker can comprise a disulfide bond, a tetrazine-trans-cyclooctene group, a sulfhydryl group, a nitrobenzyl group, a nitoindoline group, a bromo hydroxycoumarin group, a bromo hydroxyquinoline group, a hydroxyphenacyl group, a dimethozybenzoin group, or any combinations thereof.

Any art-recognized photocleavable linker can be used. In some embodiments, the cleavable linker can comprise a photocleavable linker. Generally, photocleavable linkers contain a photolabile functional group that is cleavable upon exposure to a light source (e.g., UV light) or specific wavelength. Non-limiting examples of photocleavable spacers can be found, for example, in U.S. Pat. Nos. 6,589,736 B1; 7,622,279 B2; 9,371,348 B2; 7,547,530 B2; and 7,057,031 B2; and PCT Publication No. WO2014200767, contents of all of which are incorporated herein by reference in their entirety.

In some embodiments of the various aspects described herein, the barcode composition comprises a detectable label. For example, a nucleic acid strand described herein can be modified with a detectable label, e.g., at an internal position, on the 3′- and/or 5′-end. Without wishing to be bound by a theory, such a detectable label can facilitate detection. As used herein, the term “detectable label” refers to a composition capable of producing a detectable signal indicative of the presence of a target. Detectable labels include any composition detectable by spectroscopic, photochemical, biochemical, immunochemical, electrical, optical or chemical means. Suitable labels include fluorescent molecules, radioisotopes, nucleotide chromophores, enzymes, substrates, chemiluminescent moieties, bioluminescent moieties, and the like. As such, a label is any composition detectable by spectroscopic, photochemical, biochemical, immunochemical, electrical, optical or chemical means.

A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound.

Exemplary fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green−1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CFDA; CFP—Cyan Fluorescent Protein; Chlorophyll; Chromomycin A; Chromomycin A; CMFDA; Coelenterazine; Coelenterazine cp; Coelenterazine f; Coelenterazine fcp; Coelenterazine h; Coelenterazine hcp; Coelenterazine ip; Coelenterazine 0; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green™; Oregon Green 488-X; Oregon Green™ 488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodaminelsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; and YOYO-3. Many suitable forms of these fluorescent compounds are available and can be used.

Other exemplary detectable labels include luminescent and bioluminescent markers (e.g., biotin, luciferase (e.g., bacterial, firefly, click beetle and the like), luciferin, and aequorin), radiolabels (e.g., 3H, 1251, 35S, 14C, or 32P), enzymes (e.g., galactosidases, glucorinidases, phosphatases (e.g., alkaline phosphatase), peroxidases (e.g., horseradish peroxidase), and cholinesterases), and calorimetric labels such as colloidal gold or colored glass or plastic (e.g., polystyrene, polypropylene, and latex) beads. Patents teaching the use of such labels include U.S. Pat. Nos. 3,817,837, 3,850,752, 3,939,350, 3,996,345, 4,277,437, 4,275,149, and 4,366,241, each of which are incorporated herein by reference in their entireties.

In some embodiments, the detectable label is selected from the group consisting of: fluorescent molecules, nanoparticles, stable isotopes, radioisotopes, nucleotide chromophores, enzymes, enzyme substrates, chemiluminescent moieties and bioluminescent moieties, echogenic substances, non-metallic isotopes, optical reporters, paramagnetic metal ions, and ferromagnetic metals, optionally the detectable label is a fluorophore.

Means of detecting such labels are well known to those of skill in the art. Thus, for example, radiolabels can be detected using photographic film or scintillation counters, fluorescent markers can be detected using a photo-detector to detect emitted light. Enzymatic labels are typically detected by providing the enzyme with an enzyme substrate and detecting the reaction product produced by the action of the enzyme on the enzyme substrate, and calorimetric labels can be detected by visualizing the colored label.

In some embodiments, the detectable label is a fluorophore or a quantum dot. Without wishing to be bound by a theory, using a fluorescent reagent can reduce signal-to-noise in the imaging/readout, thus maintaining sensitivity.

In some embodiments, a label can be configured to include a “smart label”, which is undetectable when conjugated with the barcode composition provided herein.

Acrydite modifications can also be made to a nucleic acid strand described herein. Acrydite modifications can permit the nucleic acid strand to be used in reactions with nucleophiles such as thiols (e.g., microarrays) or incorporated into gels (e.g., polyacrylamide). Accordingly, in some embodiments, a nucleic acid strand can comprise one or more acrydite nucleosides. The acrydite nucleoside can be at the 3′-end, 5-end, and/or at an internal position of the nucleic acid strand.

In some embodiments of the various aspects described herein, the barcode composition further comprises a nanoparticle. For example, a nucleic acid strand described herein can be conjugated with a nanoparticle, e.g., at an internal position, on the 3′- and/or 5′-end. In some embodiments, the nanoparticle is an up-converting nanoparticle. By way of example only, the up-converting nanoparticle can be utilized to perform crosslinking at different wavelengths.

In some embodiments, a nucleic acid strand describes herein can comprise a modification on the 3′ end to inhibit extension by polymerase. For example, the nucleic acid strand can comprise a ‘tail’, such as a series of T bases to prevent extension.

Any modifications to the nucleic acid strands provided herein that permit purification, extraction, quantification of expression, binding, electrophoresis, and the like, can also be made.

In some embodiments of the various aspects disclosed herein, the barcode composition further comprises primers. As used herein, the term “primer” is used to describe a sequence of DNA (or RNA) that is paired with a nucleic acid strand and provides a free 3′-OH at which a polymerase starts synthesis of a nucleic acid strand chain. Preferably, the primer is composed of an oligonucleotide. The exact lengths of the primers will depend on many factors, including temperature and source of primer. For example, depending on the complexity of the target sequence, the oligonucleotide primer typically contains 15-25 or more nucleotides, although it may contain fewer nucleotides. Short primer molecules generally require cooler temperatures to form sufficiently stable hybrid complexes with a template.

In some embodiments of any of the aspects, the barcode composition further comprises nucleotide triphosphates or deoxynucleotide triphosphates.

In some embodiments of the various aspects disclosed herein, the barcode composition further comprises a DNA or RNA polymerase. A “polymerase” refers to an enzyme that performs template-directed synthesis of polynucleotides, e.g., DNA and/or RNA. The term encompasses both the full length polypeptide and a domain that has polymerase activity. DNA polymerases are well-known to those skilled in the art, including but not limited to DNA polymerases isolated or derived from Pyrococcus furiosus, Thermococcus litoralis, and Thermotoga maritime, or modified versions thereof. Additional examples of commercially available polymerase enzymes include, but are not limited to: Klenow fragment (New England Biolabs® Inc.), Taq DNA polymerase (QIAGEN), 9° N™ DNA polymerase (New England Biolabs® Inc.), Deep Vent™ DNA polymerase (New England Biolabs® Inc.), Manta DNA polymerase (Enzymatics®), Bst DNA polymerase (New England Biolabs® Inc.), and phi29 DNA polymerase (New England Biolabs® Inc.). Polymerases include both DNA-dependent polymerases and RNA-dependent polymerases such as reverse transcriptase. At least five families of DNA-dependent DNA polymerases are known, although most fall into families A, B and C. There is little or no sequence similarity among the various families. Most family A polymerases are single chain proteins that can contain multiple enzymatic functions including polymerase, 3′ to 5′ exonuclease activity and 5′ to 3′ exonuclease activity. Family B polymerases typically have a single catalytic domain with polymerase and 3′ to 5′ exonuclease activity, as well as accessory factors. Family C polymerases are typically multi-subunit proteins with polymerizing and 3′ to 5′ exonuclease activity. In E. coli, three types of DNA polymerases have been found, DNA polymerases I (family A), II (family B), and III (family C). In eukaryotic cells, three different family B polymerases, DNA polymerases a, 6, and E, are implicated in nuclear replication, and a family A polymerase, polymerase y, is used for mitochondrial DNA replication. Other types of DNA polymerases include phage polymerases. Similarly, RNA polymerases typically include eukaryotic RNA polymerases I, II, and III, and bacterial RNA polymerases as well as phage and viral polymerases. RNA polymerases can be DNA-dependent and RNA-dependent.

It is noted that reagents, such as strand displacing DNA or RNA polymerases, and methods for synthesizing nucleic acid sequences from nucleic acid templates are well known in the art and are amenable to the invention. See, for example, US20050277146A1, US20100035303A1, and WO2006030455A1, contents of all of which are incorporated herein by reference in their entirety.

In some embodiments, the polymerase is a strand-displacing polymerase.

In some embodiments of the various aspects, the barcode composition further comprises a buffer or salt for nucleic acid synthesis. It is contemplated that buffer used in the barcode composition is chosen that permit the stability of the nucleic acids of the barcode composition. Methods of choosing such buffers are known in the art and can also be chosen for their properties in various conditions including pH or temperature of the reaction being performed

In some embodiments, two different domains can comprise identical nucleotide sequences. In some embodiments, a nucleic acid strand can comprise a restriction site. For example, the restriction site can be used within the binding regions between bound barcode strands, and a hairpin that can be ligated to cleaved ends to form a complete record strand. Alternatively, strands that bridge across junctions can be bound to the assembly and then ligated together.

The barcode composition can also include additional components and elements. For example, the barcode composition can comprise a light source for photocrosslinking and/or or cleaving, uncrosslinking, removing, or reversing a crosslink. In some embodiments, the light source is a UV light source.

In some embodiments, of the various aspects described herein, the barcode composition further comprises a target element. As used herein a “target element” refers to any molecule, compound, nucleic acid, polypeptide, lipid, antibody, or virus that can be detected by the method provided herein.

In some embodiments, the target element is immobilized on a substrate surface. In some embodiments, the target element is immobilized in a predetermined pattern. In some embodiments, the target element is an mRNA. In some embodiments, the target element is element is a nucleic acid, a lipid, a sugar, a small molecule, a microorganism or fragment thereof, a polypeptide, and/or a biological material. The biological material can be selected from tissues, tissue sections, engineered tissues, cells, patient derived cells, primary cells, organoids, extracellular matrix, 3D biological organs, dissociated cells, live cells, fixed cells, etc. . . . . Cells can be prokaryotic or eukaryotic cells.

Generally, the targeting domain of the first nucleic acid is substantially complementary to a target nucleic acid. Without limitations, the target nucleic acid can be any nucleic acid. For example, the target nucleic acid can be naturally occurring nucleic acid or a synthetic nucleic acid. It can be only a part of larger nucleic acid molecule.

Further, the target nucleic acid can be free or it can be conjugated with a target binding agent, or the target nucleic acid can be conjugated with a target molecule. Moreover, the target nucleic acid can be expressed by a target cell. Alternatively, or in addition, the target nucleic acid can be presented on a target molecule or cell, e.g., directly or indirectly via chemical crosslinking, genetic encoding, viral transduction, transfection, conjugation, cell fusion, cellular uptake, hybridization, DNA binding proteins or adaptor molecules such as target binding ligands.

In some embodiments of the various aspects disclosed herein, the target nucleic acid is conjugated with a target binding agent. As used herein a “target binding agent” means a moiety that can bind to a target element. Exemplary target binding agents include, but are not limited to, amino acids, peptides, proteins, monosaccharides, disaccharides, trisaccharides, oligosaccharides, polysaccharides, lipopolysaccharides, lectins, nucleosides, nucleotides, nucleic acids, vitamins, steroids, hormones, cofactors, receptors and receptor ligands. In some embodiments, the target binding agent is an antibody or an antigen binding fragment thereof.

In some embodiments, the target nucleic acid and/or a nucleic acid of the barcode composition provided herein is conjugated, covalently or non-covalently to a substrate, e.g., a surface of substrate. It is noted that the target nucleic acid and/or a nucleic acid of the barcode composition provided herein can be applied to any substrate surface, without the need for specialized surface treatment, such as formation of microwells common in microarray chips. Surfaces only require functionalization with nucleic acid strands which will serve as the initial docking strand of a nascent chain barcode concatemer. Alternatively, the nucleic acids can form non-covalent interactions with the substrate.

As used herein, the terms “substrate” or “substrate surface” are used interchangeably to describe a structure upon which one or more nucleic acid barcodes or concatemers of nucleic acid barcodes provided herein can be displayed or in contact with for contact with additional nucleic acids and/or labels. The nucleic acid barcodes provided herein can be conjugated to the substrate surface.

As used herein, the term “conjugated to” encompasses association of a nucleic acid with a substrate surface, a phase-changing agent or a member of an affinity pair by covalent bonding, including but not limited to cross-linking via a cross-linking agent, or by a strong non-covalent interaction that is maintained under conditions in which the conjugate is to be used.

As used herein, the term “hybridize” refers to the phenomenon of a single-stranded nucleic acid or region thereof forming hydrogen-bonded base pair interactions with either another single stranded nucleic acid or region thereof (intermolecular hybridization) or with another single-stranded region of the same nucleic acid (intramolecular hybridization). Hybridization is governed by the base sequences involved, with complementary nucleobases forming hydrogen bonds, and the stability of any hybrid being determined by the identity of the base pairs (e.g., G:C base pairs being stronger than A:T base pairs) and the number of contiguous base pairs, with longer stretches of complementary bases forming more stable hybrids. For example, hybridization between docking strands and nucleic acid barcodes comprising a photo-reactive nucleobase, e.g., CNVK base, permit the light-directed reading and/or visualization of the data stored on the substrate surface.

The substrate surface provided herein can exist in the form of a biological material (e.g., cell, tissue, or fragments thereof), platform, column, filter or sheet, dish, a microfluidic capture device, capillary tube, electrochemical responsive platform, scaffold, cartridge, resin, matrix, bead, phase changing agent, or another substrate surface known in the art. Multiple surface types can be used. Non-limiting examples of substrate surfaces include glass, transparent polymers, polystyrene, hydrogels, metal, ceramic, paper, agarose, gelatin, alginate, dextran, iron oxide, stainless steel, gold nanobeads or particles, copper, silver chloride, polycarbonate, polydimethylsiloxane, polyethylene, acrylonitrile butadiene styrene, cyclo-olefin polymers or cyclo-olefin copolymers, streptavidin, Sepharose™ resin, biological materials (e.g., cells, tissues, cell membranes, extracellular matrix proteins, etc.), and combinations thereof.

In some embodiments, the substrate can be a glass or polymer surface. In some embodiments, the substrate is a compressible hydrogel.

In some embodiments, the biological material is selected from the group consisting of: a tissue, a cell, an organoid, an engineered tissue; and an extracellular matrix.

In some embodiments, the target nucleic acid and/or the barcode composition provided herein can be applied to, or embedded within, a compressible hydrogel. In some embodiments, the target nucleic acid and/or the barcode composition provided herein represent special information, e.g., digital data and can store any information, including but not limited to text, images, graphics, movies, sequencing data, and/or health records. In some embodiments, the nucleic acid barcodes or concatemers of nucleic acid barcodes represent spatial information.

Methods of surface functionalization of these substrates with nucleic acid strands is known in the art and requires few material requirements and minimal preparation time. A typical preparation first involves passivating the surface with Bovine Serum Albumin-biotin (BSA-Biotin). The BSA binds nonspecifically with the glass surface. Secondly, a streptavidin protein will bind to the biotin attachment on the BSA protein. Finally, a biotin labeled nucleic acid can be introduced to bind to the other available binding sites on the streptavidin protein, completing the functionalization of the glass surface.

In some embodiments, the barcoding composition is modified with acrydite. Acrydite modified nucleic acid strands can be mixed with the substrate or hydrogel material and be polymerized along with the substrate or hydrogel material.

In some embodiments, the substrate is a hydrogel. A hydrogel can be naturally occurring, derived from a natural source, or derived from a synthetic source. A hydrogel can be any water-swollen and cross-linked polymeric material produced by a reaction of one or more monomers. A hydrogel can be a polymeric material that is capable of expanding to retain a significant fraction of water within its structure without dissolving into the aqueous solution. A hydrogel can also be any shrinkable material, e.g., heat-shrinkable plastics, viscoelastic foam, memory foam.

Hydrogels can be derived from natural monomeric molecules (e.g., glycosaminoglycans), hydrophilic materials (e.g., methacrylates, electrolyte complexes, vinylacetates, acrylamides), or natural polymeric materials (e.g., peptides, saccharides). Other suitable hydrogel compositions are as described in U.S. Pat. No. 6,271,278, issued Aug. 7, 2001, entitled “Hydrogel composites and superporous hydrogel composites having fast swelling, high mechanical strength, and superabsorbent properties”. Hydrogels can be comprised of hydrophobic and/or hydrophilic materials, wherein hydrophobic materials are not physically attracted to water and hydrophilic materials are physically attracted to water.

In some embodiments, a hydrogel can be a homopolymer-based hydrogel, wherein the hydrogel is derived from a single monomeric species or molecule. In some embodiments, a hydrogel can be a copolymer-based hydrogel, wherein the hydrogel is derived from two or more different monomer species or molecules. In some embodiments, a copolymer-based hydrogel is arranged in a random, block, or alternating configuration, optionally along the backbone of one of the monomers. In some embodiments, a hydrogel can be a multipolymer interpenetrating polymer-based hydrogel, wherein the hydrogel is derived from at least two different, optionally crosslinked, polymer subunits. In some embodiments, a multipolymer interpenetrating polymer-based hydrogel comprises one polymer subunit that is a crosslinked and one polymer that is a non-crosslinked polymer subunit.

A hydrogel may be non-crystalline, semicrystalline, or crystalline. A hydrogel may or may not be covalently crosslinked. A hydrogel can be synthesized using chemical methods (e.g., chemical crosslinking) or physical methods (e.g., hydrophobic interactions). A hydrogel can be neutrally charged, net positively charged, or net negatively charged. In some embodiments, a hydrogel comprises positively charged groups and negatively charged groups. In some embodiments, a hydrogel can be amphoteric or zwitterionic.

In some embodiments, a hydrogel can be pre-cast into a gel, mold, or other embedding materials before encoding with nucleic acids. In some embodiments, a hydrogel can be cast into a gel, mold or other embedding materials after encoding with nucleic acids.

The synthesis of, manipulation of, and/or addition of nucleic acids or other molecular species to a hydrogel can be facilitated using external stimuli such as electric field, magnetic field, pressure, suction and capillary action. The hydrogels provided herein can be modified for use as a biosensor (e.g., monitoring diseases, treating diseases with controlled drug release mechanisms, contact lenses, skin or mucosal tissue engraftments, or microarray disease detection). Modifications to hydrogels for use in tissue engraftments and cellular scaffolds are known in the art.

In some embodiments, microfluidics can be used to synthesize, manipulate, or add nucleic acids or other molecular species to a hydrogel.

In some embodiments, a hydrogel exists in a compressed state, wherein the hydrogel is fully compressed or shrunken and water content of the hydrogel is decreased. In some embodiments, a hydrogel exists in an expanded state, wherein the hydrogel is fully expanded, enlarged, or swelled and water content of the hydrogel is increased. In some embodiments, a hydrogel can exist in an intermediate state between fully compressed and fully expanded. In some embodiments, a hydrogel is compressed or expanded in response to changes in external environmental conditions. In some embodiments, external environmental conditions can include physical and chemical conditions, wherein physical conditions include temperature, electric potential, light, pressure, and sound, and wherein chemical conditions include pH, solvent composition (e.g., change in amount water, organic solvents), ionic strength, and small molecule solutes.

In some embodiments, biological materials such as molecules, cell-free reactions, cells, tissue sections, organoids and organisms can be immobilized on the substrate provided herein. Barcoded surfaces and substrates can be pre-patterned with a known configuration of spatial barcodes. Barcoded surfaces can be used as a grid for spatial barcoding of the biological material. Substrates can serve as docking sites for various targets in biological samples, including genomic and ribonucleic targets. Docking sites on barcoded substrates can carry functional groups, including chemical or protein tags, that can be used to bind to protein, metabolic or other targets in biological materials. Optionally, nucleic acid barcodes on the barcoded substrate can be cleaved off from the surface, using chemical, enzymatic, or photochemical methods and transferred to the biological material through diffusion or electrophoresis, force spectroscopy, or magnetic fields while preserving the overall barcode pattern.

In some embodiments of any of the aspects, the nucleic acids provided herein can be conjugated to a solid support. Without limitations, the solid support can exist in the form of a platform, column, filter or sheet, dish, a microfluidic capture device, capillary tube, electrochemical responsive platform, scaffold, cartridge, resin, matrix, bead, or another solid support known in the art.

In some embodiments, the solid support comprises materials that include, but are not limited to, a polymer, metal, ceramic, gels, paper, or glass. The materials of the solid support can further comprise, as non-limiting examples, polystyrene, agarose, gelatin, alginate, iron oxide, stainless steel, gold nanobeads or particles, copper, silver chloride, polycarbonate, polydimethylsiloxane, polyethylene, acrylonitrile butadiene styrene, cyclo-olefin polymers or cyclo-olefin copolymers, or Sepharose™ resin.

In some embodiments, the solid support can further comprise a magnetoresponsive element such as a magnetoresponsive bead. In some embodiments, the magnetoresponsive element or bead is in the form of a sphere, cube, rectangle, cylinder, cone, or any other shape described in the art.

In some embodiments, the magnetoresponsive element comprises magnetite, iron (III) oxide, samarium-cobalt, terfenol-D, or any other magnetic element described in the art.

In some embodiments, the substrate comprises a predetermined pattern of target elements or nucleic acids.

In some embodiments, the substrate does not have a pre-determined pattern of target nucleic acids. For example, the spatial information of the target nucleic acid (e.g., a biomarker) may be unknown prior to hybridization with the barcoding composition.

### Methods

Also provided herein are methods for barcoding or detecting a target element.

In one aspect, the method comprises: (a) hybridizing a target mRNA (a first nucleic acid) with a second nucleic acid, and wherein: (i) the mRNA comprises a first hybridization domain comprising a polyA sequence; and (ii) the second nucleic acid comprises in a 5′ to 3′ direction: (1) a second hybridization domain, wherein the second hybridization domain is substantially complementary to the first hybridization domain and comprises a photoreactive element; and (2) a first barcode domain, and (b) photocrosslinking the mRNA with the second nucleic acid thereby forming a probe-primer complex; (c) synthesizing a record nucleic acid from the probe-primer complex; and (d) detecting the record nucleic acid.

In another aspect, the method comprises: (a) hybridizing a target nucleic acid with a first nucleic acid and hybridizing a second nucleic acid with the first nucleic acid, wherein: (i) the first nucleic acid comprising in a 5′ to 3′ direction: (1) optionally, a unique molecule identifier (UMI) sequence; (2) a first targeting domain substantially complementary to a nucleic acid of the target element; and (3) a first hybridization domain; and (ii) the second nucleic acid comprising in a 5′ to 3′ direction: (1) a second hybridization domain, wherein the second hybridization domain is substantially complementary to the first hybridization domain; and (2) a first barcode domain, and wherein at least one of the first or second hybridization domain comprises a photoreactive element; (b) photocrosslinking the first nucleic acid with the second nucleic acid thereby forming a probe-primer complex; (c) optionally, denaturing the probe-primer complex from the target nucleic acid; (d) synthesizing a record nucleic acid from the probe-primer complex; and (e) detecting the record nucleic acid.

In another aspect, the method comprises: (a) hybridizing a target mRNA (a first nucleic acid) with a second nucleic acid, and wherein: (i) the mRNA comprises a first hybridization domain comprising a polyA sequence; and (ii) the second nucleic acid comprises in a 5′ to 3′ direction: (1) a second hybridization domain, wherein the second hybridization domain is substantially complementary to the first hybridization domain of the mRNA and comprises a photoreactive element; and (2) a first barcode domain, and (b) photocrosslinking the mRNA with the second nucleic acid thereby forming a first complex; (c) hybridizing a third nucleic acid to the second nucleic in the first complex thereby forming a probe-primer complex, wherein the third nucleic acid comprises a second barcode domain substantially complementary to the first barcode domain of the second nucleic acid; (d) synthesizing a record nucleic acid from the probe-primer complex; and (e) detecting the record nucleic acid.

In another aspect, the method comprises: (a) hybridizing a target nucleic acid with a first nucleic acid and hybridizing a second nucleic acid to the first nucleic acid, wherein: (i) the first nucleic acid comprises in a 5′ to 3′ direction: (1) optionally, a unique molecule identifier (UMI) sequence; (2) a first targeting domain, wherein the first targeting domain is substantially complementary to the target nucleic acid; and (3) a first hybridization domain; and (ii) the second nucleic acid comprises in a 5′ to 3′ direction: (1) a second hybridization domain, wherein the second hybridization domain is substantially complementary to the first hybridization domain of the first nucleic acid; and (2) a first barcode domain, and wherein at least one of the first or second hybridization domain comprises a photoreactive element; and (b) photocrosslinking the first nucleic acid with the second nucleic acid thereby forming a first complex; (c) optionally, denaturing the first complex from the target nucleic acid; (d) hybridizing a third nucleic acid to the second nucleic acid in the first complex thereby forming a probe-primer complex, wherein the third nucleic acid comprises a second barcode domain substantially complementary to the first barcode domain of the second nucleic acid; (e) synthesizing a record nucleic acid from the probe-primer complex; and (f) detecting the record nucleic acid.

In another aspect, the method comprises: (a) hybridizing a target nucleic acid with a first nucleic acid, wherein: (i) the first nucleic acid comprises in a 5′ to 3′ direction: (1) optionally, a unique molecule identifier (UMI) sequence; (2) a first targeting domain, wherein the first targeting domain is substantially complementary to the target nucleic acid; and (3) a first hybridization domain; (b) preparing a concatemer by hybridizing n additional nucleic acids and photocrosslinking the additional nucleic acids with the first complex, wherein n optionally is an integer from 1 to 100, and wherein each additional nucleic acid comprises in 5′ to 3′ direction: (i) a first hybridization domain; (ii) a barcode domain; and (iii) a second hybridization domain, and wherein the first hybridization domain of nth nucleic acid is substantially complementary to the second hybridization domain of (n−1)th nucleic acid, wherein the first hybridization domain of n=1 nucleic acid is substantially complementary to the first hybridization domain of the first nucleic acid, and wherein at least one of the first or second hybridization domain of each nucleic acid comprises a photoreactive element; (c) hybridizing a first cap nucleic acid with the concatemer thereby forming a capped concatemer, wherein the first cap nucleic acid comprises: (i) a first cap hybridization domain, wherein the first cap hybridization domain is substantially complementary to the second hybridization domain of nth nucleic acid; and (ii) a second cap hybridization domain; (d) hybridizing a second cap nucleic acid to the capped concatemer thereby forming a concatemer-primer complex, wherein the second cap nucleic acid comprises in a 5′ to 3′ direction: (i) a primer sequence domain; (ii) optionally, a unique molecular identifier (UMI) sequence; and (iii) a hybridization domain, wherein the hybridization domain is substantially complementary to the second cap hybridization domain of the first cap nucleic acid, and wherein at least one of the second hybridization domain of the first cap hybridization domain of the second cap nucleic acid comprises a photoreactive element; and (e) detecting the concatemer-primer complex or synthesizing a record nucleic acid from the concatemer-primer complex and detecting the record nucleic acid.

In another aspect, the method comprises: (a) hybridizing a target nucleic acid strand in each member the plurality of targets with a first nucleic acid strand, wherein the target nucleic acid strand is different in each member the plurality of targets, wherein the target nucleic acid strand is comprised within another nucleic acid molecule, or the target nucleic acid strand is conjugated with a member of the plurality of targets, or the target nucleic acid strand is expressed by a cell, or the target nucleic acid strand is presented on a target or cell directly or indirectly via chemical crosslinking, genetic encoding, viral transduction, transfection, conjugation, cell fusion, cellular uptake, hybridization, DNA binding proteins or a target binding agent/ligand, and wherein: (i) the first nucleic acid strand comprises in a 5′ to 3′ direction: (1) optionally, a unique molecule identifier (UMI) sequence; (2) a first targeting domain, wherein the first targeting domain is substantially complementary to the target nucleic acid; and (3) a first hybridization domain; (b) preparing a concatemer by hybridizing in a stepwise manner one or more additional nucleic acid strand and photocrosslinking the additional nucleic acid strands with the first complex, wherein said photocrosslinking comprises selecting predetermined regions of the sample and exposing the predetermined regions to light after hybridizing each additional nucleic acid strand thereby cross-linking the complementary hybridization domains, and removing any non-crosslinked additional nucleic acid strands after exposure to light and prior to hybridization a next additional nucleic acid strand, and wherein each additional nucleic acid strand comprises in 5′ to 3′ direction: (i) a first hybridization domain; (ii) a barcode domain; and (iii) a second hybridization domain, and wherein the first hybridization domain of nth additional nucleic acid strand is substantially complementary to the second hybridization domain of (n−1)th additional nucleic acid strand, wherein the first hybridization domain of the first additional nucleic acid strand is substantially complementary to the first hybridization domain of the first nucleic acid strand, and wherein at least one of the first or second hybridization domain of each nucleic acid strand comprises a photoreactive element; and (c) detecting the concatemer and/or synthesizing a record nucleic acid from the concatemer and detecting the record nucleic acid.

In various embodiments of the aspects provided herein, the methods comprise preparing a biological sample. Sample preparation can include obtaining a biological sample from a subject. Sample preparation can also include culturing cells, tissues, and organoids by methods known in the art. In some embodiments, the sample is imaged. In some embodiments, the sample undergoes live cell imaging. In some embodiments, the sample is fixed and permeabilized for imaging. The amount of time that a sample is prepared can be determined by the skilled artisan.

In various embodiments of the aspects provided herein, the methods comprise imaging and barcoding a target nucleic acid in a sample. The sample provided herein can undergo in situ reverse transcription, A-tailing, and optionally, in situ hybridization (ISH), immunofluorescence (IF), or other immunohistochemical methods.

In various embodiments of the aspects provided herein, the methods comprise photocrosslinking two or more nucleic acid strands. The photocrosslinking can be performed under any needed conditions. In some embodiments, photocrosslinking can be performed in aqueous solution.

The light used for photocrosslinking will be dependent on the photoreactive elements. Generally, photocrosslinking is using a 350-400 nm wavelength of light. Preferably, photocrosslinking is using a light source with a wavelength of about 365 nm.

In some embodiments, the methods further comprise one or more wash steps, e.g., to wash away any remaining reagent and/or nucleic acid strands.

In some embodiments of the various methods described herein, the target element, e.g., the target nucleic acid can be conjugated with a target binding ligand. For example, the target nucleic acid can be conjugated with a target binding element for binding to the actual target element to be barcoded and/or detected.

In some embodiments of the various methods described herein, the target nucleic acid is comprised in a biological material. For example, the target nucleic acid can be expressed by a target cell, the target nucleic acid can be presented on a target molecule or cell, e.g., directly or indirectly via chemical crosslinking, genetic encoding, viral transduction, transfection, conjugation, cell fusion, cellular uptake, hybridization, DNA binding proteins or adaptor molecules such as target binding ligands.

In some embodiments of the various methods described herein, the target element, e.g., the target nucleic acid is immobilized on a substrate surface. The target element, e.g., the target nucleic acid can be immobilized on the substrate surface in a predetermined pattern.

In some embodiments, the methods further comprise selecting one or more specific regions of interest for illumination or detection. The selection can be manual or computer aided. Generally, the selection is based on one or more phenotypic markers. Exemplary phenotypic markers for selecting one or more specific regions of interest for illumination or detection include, but are not limited to fluorescence, shape, or morphology. In some embodiments, the phenotypic marker is fluorescence, shape, intensity, histological stains, antibody staining, or morphology.

Some embodiments of the various aspects described herein further comprise software for automatically detecting and processing one or more regions of interest for spatial illumination or detection.

In various embodiments of the aspects provided herein, the methods comprise record strand extraction and sequencing. The record extraction can be performed by RNase H displacement and/or in situ or in vitro hopPER synthesis. In some embodiments, the strands can be purified by column or bead-based purification methods known in the art. The strands can then be amplified for detection and/or sequencing by PCR. Optionally, amplicons can be purified along with secondary amplification steps and/or adaptor ligation for library preparation. Optionally, rRNA can also be reduced by methods known in the art.

In some embodiments of any of the aspects, the method can be applied to the 5′ end of a synthesized cDNA library.

In some embodiments, the method can utilize a photoreactive agent to serve as a blocking domain. In some embodiments the photoreactive agent is CNVK.

Exemplary methods for detecting the record strand include, but are not limited to sequencing the record nucleic acid, light microscopy, high throughput scanner, confocal microscopy, light sheet microscopy, electron microscopy, atomic force microscopy, and/or the unaided eye.

In some embodiments of any of the aspects, the method further comprises amplifying the record strand, e.g., prior to detection. As used herein, the term “amplifying” refers to a step of submitting a nucleic acid sequence to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid, such as is obtained with cycle sequencing. Methods of amplifying and synthesizing nucleic acid sequences are known in the art. For example, see U.S. Pat. Nos. 7,906,282, 8,367,328, 5,518,900, 7,378,262, 5,476,774, and 6,638,722, contents of all of which are incorporated by reference herein in their entirety.

In some embodiments, amplifying the record strand comprises a polymerase chain reaction (PCR). PCR is well known to those of skill in the art; see, e.g., U.S. Pat. Nos. 4,683,195 and 4,683,202; and PCR Protocols: A Guide to Methods and Applications, Innis et al., eds, 1990, contents of all which are incorporated herein by reference in their entirety. Exemplary PCR reaction conditions typically comprise either two or three step cycles. Two step cycles have a denaturation step followed by a hybridization/elongation step. Three step cycles comprise a denaturation step followed by a hybridization step followed by a separate elongation step.

In some embodiments, the amplification step includes additional polynucleotide sequences or templates with hairpins that are orthogonal the amplification step. Without wishing to be bound by a theory, such additional DNA hairpins can reduce or correct for off-target reactions. For example, when a three-letter code is used, these additional hairpin comprising sequences or templates can serve to soak up the trace amounts of unwanted nucleotide that can be present in some samples.

In some embodiments, a photocrosslink linking two nucleic acid strands can be cleaved, uncrosslinked, removed or reversed prior to amplifying and/or sequencing the record strand. The photocrosslink can be cleaved, uncrosslinked, removed or reversed using a light using a light source with a wavelength of about 315 nm.

A record strand can be read using a nucleic acid sequencing technology. In some embodiments, the sequence of the record strand can be determined through the use of complementary sequences labeled with detectable moieties such as fluorophores, quantum dots, peptide tags, beads (e.g., agarose, latex, magnetoresponsive, chromatic), polymer dots, nanoparticles, additional docking sites, tags such as biotin, or functional groups such that their presence may be detected e.g., by fluorescence microscopy, fluorescent scanners, optical scanners and the like.

In some embodiments of any of the aspects provided herein, the method comprises barcoding biomolecules in pre-defined regions of interest. For example, whole tissues, tissue regions, collection of cells, single cells, subcellular regions, microbes, and surfaces. In order to tag each region for multimodal integrated analysis, imaging based methods and/or sequencing can be used as described above.

In some embodiments of any of the aspects provided herein, the method comprises barcoding biomolecules to create spatial tags that relate sequencing reads back to spatial positions for multimodal integrated analysis of selected regions of interest.

The methods provided herein can be used for screening libraries of candidate treatments for various diseases and disorders (e.g., small molecule drugs, biologics, therapeutic nucleic acids, gene or cell therapies, siRNAs, gRNAs, plasmids, phages, viruses, peptides, proteins, antibodies, metabolites, hormones, DNA encoded libraries). In some embodiments, phenotypic outcomes are identified by imaging. Selected regions are can be barcoded by light exposure for sequencing based analysis using the method provided herein.

The method provided herein can be used to identify novel therapies and diagnostics for various diseases and disorders. Small molecule drugs, biologics, therapeutic nucleic acids, gene or cell therapies, siRNAs, gRNAs, peptides, proteins, antibodies, metabolites, hormones, DNA encoded libraries can be screened to identify drug targets and/or biomarkers. Non-limiting examples of applications for the methods provided herein include drug screening, biomarker identification, profiling, characterization of phenotypic to genotypic cell state, generation of new disease models, characterization of cells and disease models, characterization of differentiation status and cell state, tissue mapping, multi-dimensional analysis, high content screening, machine-learning based clustering or classification, cell therapy development, CAR-T therapy development, antibody screening, personalized medicine, and cell enrichment.

### Devices

The methods described herein can be performed on a device. For example, a method described herein can be performed on a device comprising a light source and a sample holder. In some embodiments, a method described herein can be performed on a device comprising a light source, an optical mask or digital micromirror device and a sample holder, and optionally one or more lenses for focusing light. In some embodiments, a method described herein can be performed on a device comprising a light source, an optical mask or digital micromirror device, a sample holder and a fluidic or microfluidic system, wherein the device is configured for automation. In some embodiments, a method described herein can be performed on a device comprising a fluidic system configured to deliver the barcode composition onto a sample in predefined steps. In some embodiments, a method described herein can be performed on a device comprising a light source, an optical mask or digital micromirror device, a camera, a fluidic or microfluidic system and a set of software tools, wherein the device is configured for automatically identifying cells and/or barcode assignments.

In some embodiments, a method described herein can be performed on a device comprising a sensor, wherein the device is configured to respond to a signal from a method described herein and adjust/modulate delivery of the barcode composition. In some embodiments, a method described herein can be performed on a device comprising a sensor and a fluidic device, wherein the device is configured to respond to external input from one or more acquired images and/or a signal from a method described herein and adjust/modulate delivery of the barcode composition.

It is noted that barcode composition described herein can be included in device. For example, a device can comprise a barcode composition described herein and the device comprises a delivery mechanism for the barcode composition onto a sample in predefined steps for automation. In some embodiments, a device described herein comprises a sample holder, where the sample holder is configured for automated delivery of a barcode composition described herein. In some embodiments, a device described herein comprises a sample holder, where the sample holder is configured for securing a barcode composition described herein. A device comprising a barcode composition described herein can be configured for attaching to and/or augmenting existing devices and workflows.

In some embodiments, a device can comprise a reservoir for holding one or more components of a barcode composition described herein. For example, the device can comprise a reservoir for holding a nucleic acid strand comprising a photoreactive element, e.g., a CNVK-modified barcoding strand.

In another aspect, provided herein is a device for use in a method provided herein, wherein the device comprises a light source and a sample holder. In some embodiments, the device comprises a barcode composition provided herein in the sample holder.

In some embodiments, the device further comprises an optical mask or Digital micromirror device. In some embodiments, the device further comprises at least one lens for focusing light. In some embodiments of any of the aspect, the light source provided herein the light source is a UV light source, a lamp, a LED, at least one laser or a two photon laser with or without modulation through a lens system, a photomask, a digital micromirror device, a pinhole and/or a structured illumination.

In some embodiments, the device comprises a housing. In some embodiments, the device further comprises a fluidic or microfluidic system. In some embodiments, the device comprises a fluidic or microfluidic system for delivering a composition provided herein to the sample holder in predefined steps. Microfluidic systems are known in the art and are described, e.g., in U.S. application Ser. Nos. 16/125,433; 16/134,746; U.S. Pat. Nos. 9,694,361 B2; 5,876,675 A; 6,991,713 B2; and WO2001/045843A2, which are incorporated herein by reference in their entireties.

In some embodiments, the device further comprises a detector. In some embodiments, the device further comprises a camera.

In some embodiments, the device comprises components for processing the barcodes detected by the methods provided herein. In some embodiments, the device comprises software for automatically identifying cells and/or barcode assignments.

In some embodiments, the device comprises a reservoir containing a crosslinkable strand. In some embodiments, the device comprises a reservoir containing CNVK-modified barcoding strands.

In some embodiments, the device provided herein has automated features that permit the delivery of the compositions provided herein.

In some embodiments, the device comprises a sample holder designed to secure the compositions provided herein.

In some embodiments, the device comprises a sensor. In some embodiments, the device comprises a sensor, a fluidic device that responds to external input from acquired images, detected signal provided herein and adjusts delivery of the compositions provided herein.

In some embodiments, the device is attached to a microscope and/or a computer system.

### Definitions

For convenience, the meaning of some terms and phrases used in the specification, examples, and appended claims, are provided below. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects provided herein, and are not intended to limit the claimed invention, because the scope of the invention is limited only by the claims. Further, unless otherwise required by context, singular terms shall include pluralities and plural terms shall include the singular.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, “nucleic acid” means DNA, RNA, single-stranded, double-stranded, or more highly aggregated hybridization motifs, and any chemical modifications thereof.

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those provided herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean ±1%.

The term “substantially identical” means two or more nucleotide sequences have at least 65%, 70%, 80%, 85%, 90%, 95%, or 97% identical nucleotides. In some embodiments, “substantially identical” means two or more nucleotide sequences have the same identical nucleotides.

As used herein the term “complementary” generally refers to the potential for a hybridized pairing or binding interaction between two sets of nucleic acids. Complementary nucleic acids are capable of binding to one another through hydrogen bond pairing according to canonical Watson-Crick base pairing and non-Watson-Crick base pairing (e.g., Wobble base pairing and Hoogsteen base pairing). In some embodiments, two sets of nucleic acids may be 100% complementary to one another. In other embodiments, two sets of nucleic acids may comprise 1, 2, 3, 4, 5, 6, 7, 8, 9, 10, or more nucleotides that are not complementary. In other embodiments, two sets of nucleic acids may be at least 50%, at least 60%, at least 70%, at least 80%, or at least 90% complementary. In some embodiments, two sets of nucleic acids are complementary so long as they are capable of forming a stable or transient complex. “Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but not limited to, G:U Wobble or Hoogsteen base pairing.

As used herein, the term “hybridization domain(s)” generally refers to either a portion of a first nucleic acid or a second nucleic acid, wherein the second hybridization domain of the second nucleic acid is substantially complementary to the first hybridization domain of the first nucleic acid. In some embodiments, a hybridization domain is a photoreactive strand, as defined herein. In some embodiments, a hybridization domain is a complementary strand, as defined herein. In some embodiments, two alternating hybridization domains refer to a single crosslinking strand and a single complementary strand.

As used herein, the term “probe domain” or “targeting domain” generally refers to a portion of the first nucleic acid that is complementary to the target element.

As used herein, an “attachment nucleic acid strand” refers to any nucleic acid that allows for the nucleic acids provided herein to associate with, crosslink to, embed into, or tether to, covalently or non-covalently interact with the another nucleic acid or a substrate provided herein. In some embodiments, the attachment nucleic acid strand comprises a barcode domain and a hybridization domain, wherein the hybridization domain optionally comprises a photoreactive element. In some embodiments, the attachment nucleic acid strand is substantially complementary to at least part of the first nucleic acid.

As used herein, a “barcode domain,” refers to the part of the barcode strand that comprises a nucleic acid sequence that represents spatial, sequencing information, and/or and encodes data. The barcode domain sequence can be predetermined by a barcode library. The barcode domain can be a sequence comprising DNA, RNA, synthetic nucleobases, or any combination thereof. A barcode domain can be assigned a bit value. For example, each barcode domain can be independently assigned a bit value. It is noted that bit values are not limited to 0 and 1. A nucleic acid strand comprising a barcode domain can also be referred to as a barcode strand herein.

As used herein, the term “barcode library” is a collection of stored nucleic acid sequences with associated information. Each sequence and the associated information are stored in a database with information such as the sequence, pattern, structure, and label. The barcode library can be used to decipher or read the special information contained in each barcode strand. The barcode library can also be used to pre-determine the concatemer pattern for data storage, writing, and reading of the concatemers. In some embodiments, the barcode domain of the first and/or second nucleic acid is selected from a barcode library having a minimum Hamming distance of 4.

As used herein, the term “nucleic acid concatemer” generally refers to a nucleic acid that comprises at least three nucleic acid barcodes. A nucleic acid concatemer may comprise nucleic acid barcodes that are covalently linked to one another via photoreactive nucleotides. In some embodiments, a nucleic acid concatemer may comprise at least 1, at least 2, at least 3, at least 4, at least 5, or at least 10 nucleic acid barcodes. In some embodiments, a nucleic acid concatemer may comprise at least 1, at least 2, at least 3, at least 4, at least 5, or at least 10 barcode strands that each incorporate data, e.g., each barcode strand may uniquely/independently be assigned spatial or sequencing information.

As used herein, the term “spatial information” is any information, coordinates, markers in a biological tissue or matrix, that can be stored in the barcode. The spatial information can inform one of skill in the art where on the substrate a particular marker, barcode, or pattern is located. For example, spatial information may be useful in creating an image or QR code with the nucleic acid barcodes. Spatial information can also be useful in the detection of a specific nucleic acid target.

As used herein, the term “agent” refers to any substance, chemical constituent, chemical molecule of synthetic or biological origin.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

### EXAMPLES

### Example 1: Light-Directed Biomolecular Barcoding Summary

Single-cell sequencing has revealed critical new understandings of biology by providing quantitative cell-level transcriptomics information. But multi-scale spatial information, both at the sub-cellular level and the level of cells positioned within a tissue, is lost in the process of dissociating cells for cell level sequencing. Provided herein is a method for light-directed barcoding followed by sequencing, that allows for programmable labeling of immobilized biomolecules across length scales (sub-cellular to large tissues) with barcode sequences that attach to immobilized sequences in situ. The concatenated barcode and in situ sequences can be read out with next-generation sequencing platforms to provide combined sequence and spatial information.

To understand how cells function, differentiate and respond to environmental factors, high-throughput methods that enable profiling molecular states of single cells in their native environment are necessary. Next generation sequencing methods allow characterizing the cell diversity by simultaneous detection of thousands of distinct transcripts from cell populations. More recently, these approaches have been further extended for transcriptomic profiling of individual cells by single-cell RNA-Seq (scRNA-Seq) methods like Drop-Seq that rely on tracing the transcript information back to isolated cells or nuclei. The sequencing readouts can then be used to define cell types and states by clustering of read profiles. These methods, however, require special instruments like cell sorters, microwells or custom microfluidics, and offer limited throughput. More importantly, the reads obtained inherently lack the spatial information that would allow linking the molecular profiles to the original location of the individual cells in the tissue, as well as subcellular localization of the molecules of interest within these cells.

Direct imaging of samples with microscopy as in single-molecule FISH (smFISH) offers to reconcile sequence information with spatial context. However, FISH approaches suffer from low signal to background and low multiplexing. To improve the signal level for reliable detection of RNAs in tissue samples with high autofluorescence, and scattering, several studies integrated FISH with signal amplification that improves the fluorescence per spot but localizing multitudes of fluorescent oligonucleotides on the same target using approaches like rolling circle amplification (RCA), hybridization chain reaction (HCR), branched DNA assays (bDNA), signal amplification by exchange reaction (SABER) or clampFISH.

Due to spectral overlap multiplexed analysis of the same sample is also quite limited, allowing only low-plex (3-4 targets at a time) investigations. Multiplexing limits have been overcome via iterative exchange rounds of fluorophores or probes, combinatorial fluorescence barcoding or in situ sequencing. Whereas exchange-imaging methods are time-consuming to scale-up, methods that rely on combinatorial fluorescence labeling or in situ sequencing require the targets to be spatially separated and resolvable as unique puncta, hence generally perform more reliably for low abundance transcripts. This places an upper limit on the number of reads obtainable per cell, and leads to poor detection sensitivity, especially when the noise and bias coming from in situ enzymatic reactions, and limitations of in situ sequencing related to read-depth, read-length and base-calling errors are taken into account. Even with the most recent improvements, the detection efficiency of these methods have been <50% of smFISH. While pairing the combinatorial labeling methods with super-resolution approaches like localization microscopy and expansion microscopy further provide super-resolution information, data acquisition becomes inhibitively slow as imaging times are long and scale with volume. Furthermore, as optical elements have a strong influence on the final result, setup to setup variation of the optical elements like cameras, objectives, pinholes, light sources as well as use of different fluorophores for imaging assays change aspects like light collection, noise, chromatic aberration, flatness of the illumination field, out-of-focus fluorescence, spectral bleed-through, photobleaching, quenching.

An emerging strategy for combining spatial information with single-cell sequencing techniques is to utilize oligonucleotide capture arrays or surfaces pre-barcoded via printing or linking unique DNA sequences (i.e. DNA barcodes) per spatial position. These DNA barcodes are then associated with the molecules of interest in the vicinity of each barcoded spatial position, and are finally sequenced to retrieve and map the spatial information for each captured target. Other recent advancements allow a partial retrieval of the subcellular distribution information of transcripts based on proximity to molecular landmarks like organelles, differential permeabilization of cellular membranes, or processing stages of RNAs. RNA transcript and genomic reads can also be grouped by proximity to each other, using methods that physically link nearby sequences together.

To address all these limitations collectively, a light-based spatial barcoding and high-throughput sequencing strategy was developed that encodes the spatial information directly on each target molecule in situ without the need for pre-patterned capture arrays and without destruction of the sample. Provided herein is a method of DNA photolithography used to selectively crosslink barcode strands to target molecules in specified spatial positions.

The method provided herein reconciles the power of high-throughput and highly multiplexed next generation sequencing with the detection sensitivity and sampling efficiency of FISH in a scalable manner, while preserving the absolute spatial information with subcellular resolution for each target molecule. It complements existing single-cell sequencing methods and allows probing of the samples at desired levels of resolution with the possibility to further define areas of interest based on markers. This additional flexibility can also be used to achieve a FACS-like sorting in situ without dissociation of the cells or proximity-based labeling of subsets of molecules in close vicinity of functional or spatial markers.

The Method: The fundamental strategy for the light directed biomolecular barcoding methods provided herein leverage fast DNA crosslinking chemistry and spatially confined light patterns to spatially address and print DNA barcodes in a massively parallelized fashion. This crosslinking design is sequence specific and reversible, which enables unique crosslinking geometries that can be engineered for barcode retrieval.

### Example 2: Reaction Chemistries for Barcoding

Strategy 1: Dual Light-Directed Barcoding:

The first strategy utilizes two wavelengths of light to crosslink (˜365 nm) primers to probes/transcripts of interest, followed later by a crosslinking reversal step (˜312 nm), see FIG. 1A-1D. In a targeted approach, probes designed to be complementary to genomic, transcriptomic sequences, or other sequences of interest are hybridized in situ (Fig. FIG. 1A). A secondary hybridization step binds a primer that contains a CNVK modification in the region complementary to the probe, in addition to additional domains on the 5′ end including a forward primer (For), optionally a unique molecular identifier (UMI), and a barcode sequence (in purple). Upon illumination under UV light (approximately 365 nm), the primer becomes covalently linked (crosslinked) to the probe sequence, and a polymerase is used to copy the full record strand. This may be done after the probe-primer complex is denatured from the sample, or a strand displacing polymerase may be used to displace the record strands in situ. Crosslinking is reversed using UV light at approximately 312 nm. Records strands may be PCR amplified before ultimately being sequenced to recover combined barcode/UMI and probe sequence/identity information.

The targeted approach can also be used to bind other nucleic acids immobilized in a sample or on a surface, such as DNA-conjugated antibodies bound to protein targets of interest (FIG. 1B). In general, any entity that can be labeled with or crosslinked to a strand of interest can be recorded with this strategy.

In a non-targeted approach, primers are bound to conserved or abundant sequences in targets of interest. For example, mRNAs with polyA sequences on their 3′ ends may be bound to barcode-containing primers via a complementary CNVK-containing sequence domain comprising one or more polyT sequences (FIG. 1C). The primer contains a primer domain (For), optionally a unique molecular identifier (UMI) domain, a barcode domain (Barcode/Bar), in addition to the CNVK-containing domain. A reverse transcriptase enzyme can then be used to extend the primer to copy the mRNA sequence before or after crosslinking is reversed. Record sequences containing combined barcode and mRNA sequence information are then prepared for sequencing with standard methods, for example by utilizing a template switching oligo (TSO) that appends a primer on the 3′ end of the record strand to enable PCR amplification. Sequencing of records is used to recover combined RNA transcript and barcode sequence data. Other types and/or portions of RNA and DNA molecules can be examined through the use of primer libraries and/or primers with random sequences.

Strategy 2: Light-Directed Barcoding with Bridge Sequences:

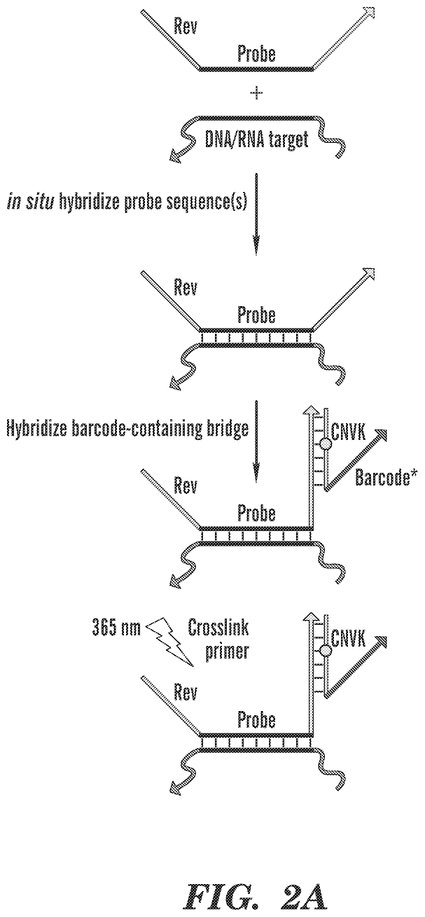

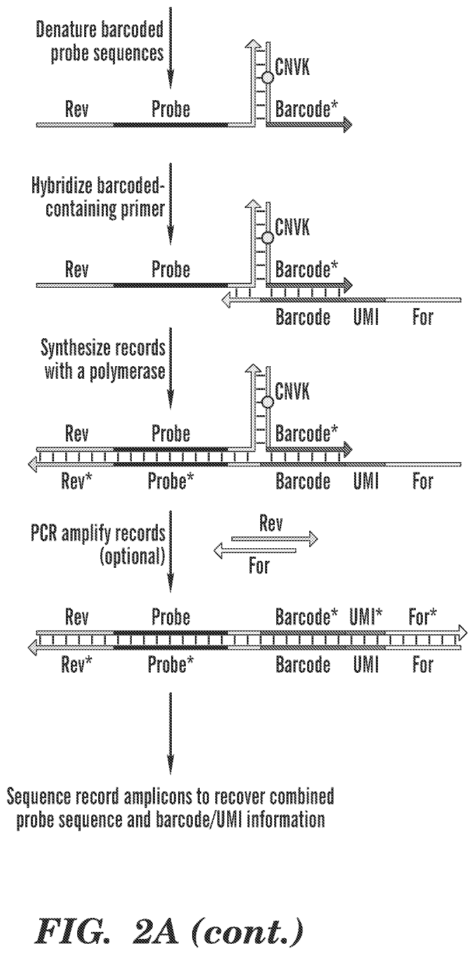

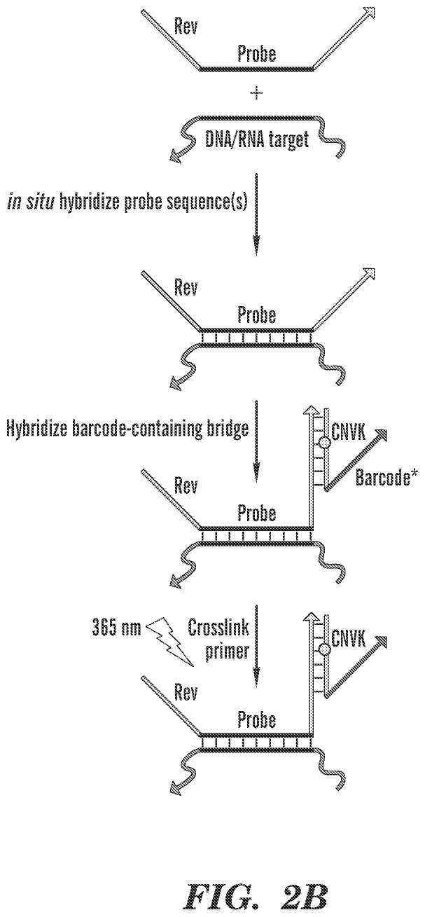

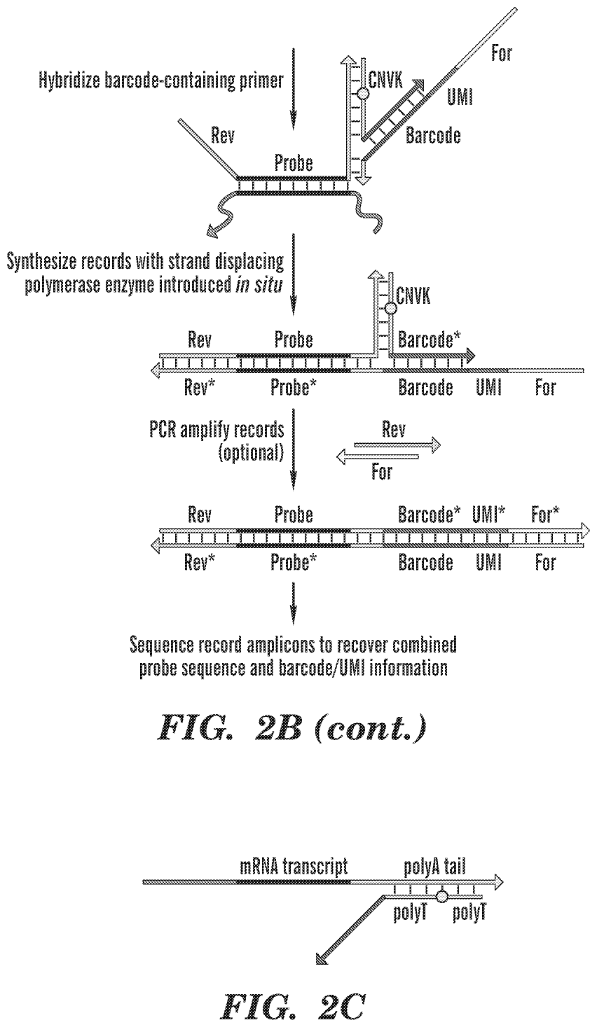

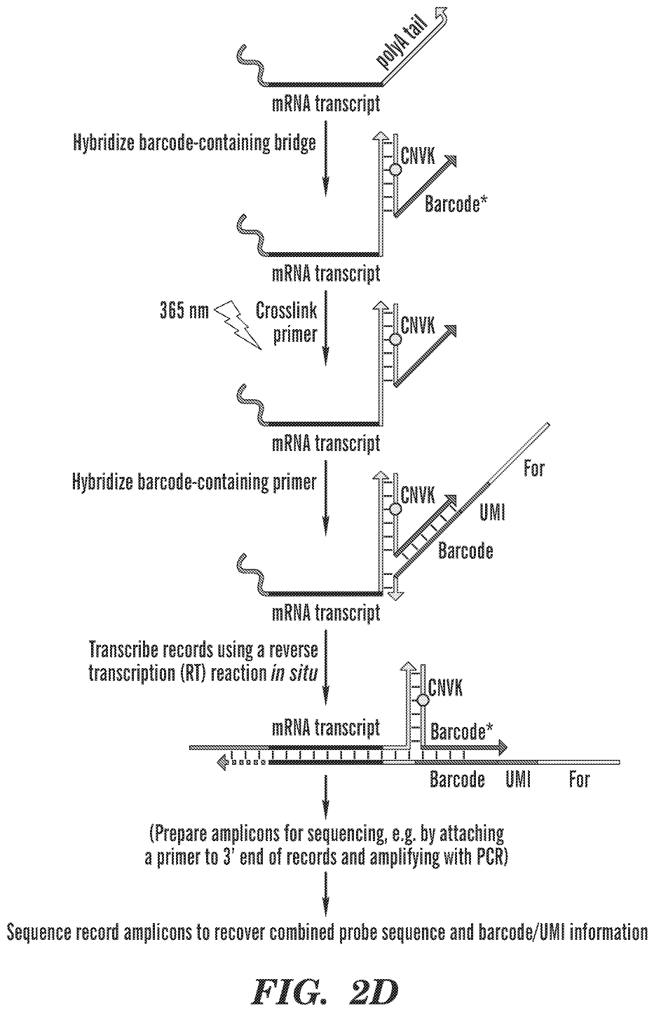

The second strategy uses only a single wavelength of light (˜365 nm) for crosslinking of CNVK-containing sequences to semi- or fully-complementary sequences and a bridge sequence to avoid the need for crosslinking reversal, see FIG. 2A-2D.

In a targeted approach, probes designed to be complementary to genomic or transcriptomic sequences of interest are hybridized in situ (FIG. 2A-2B). A secondary hybridization step binds a bridge sequence that contains a CNVK modification in the region complementary to the probe, in addition to a barcode domain (Barcode*). Upon illumination under UV light (approximately 365 nm), the bridge becomes covalently linked (crosslinked) to the probe sequence. After denaturing of probe-bridge complexes, a primer is hybridized. This primer contains a forward primer sequence (For), optionally a unique molecular identifier (UMI), a barcode sequence (Barcode) complementary to the bridge, and a short 3′ overhang complementary to the probe overhang to allow it to reach across the probe-bridge junction (FIG. 2A). A polymerase is used to copy the full record strand. Records strands may be PCR amplified with the forward (For) and reverse (Rev) primers before ultimately being sequenced to recover combined barcode/UMI and probe sequence/identity information. If a strand displacing polymerase is used to copy the record, then the denaturing step can be skipped, and the primer may be hybridized to the probe-bridge complex directly in situ (FIG. 2B).

In a non-targeted approach, bridges are bound to conserved or abundant sequences in targets of interest. For example, mRNAs with polyA sequences on their 3′ ends may be bound to barcode-containing bridges via a complementary CNVK-containing sequence domain comprising one or more polyT sequences (FIG. 2C-2D). The primer contains a primer domain (For), optionally a unique molecular identifier (UMI) domain, and a barcode domain (Barcode) that binds the barcode domain on the bridge strand (Barcode*). A reverse transcriptase enzyme can then be used to extend the primer to copy the mRNA sequence before or after crosslinking is reversed. Record sequences containing combined barcode and mRNA sequence information are then prepared for sequencing with standard methods, for example by utilizing a template switching oligo (TSO) that appends a primer on the 3′ end of the record strand to enable PCR amplification. Sequencing of records is used to recover combined RNA transcript and barcode sequence data. Other types and/or portions of RNA and DNA molecules can be examined through the use of primer libraries and/or primers with random sequences.

Strategy 3: Light-Directed Barcoding with Concatemer Assembly:

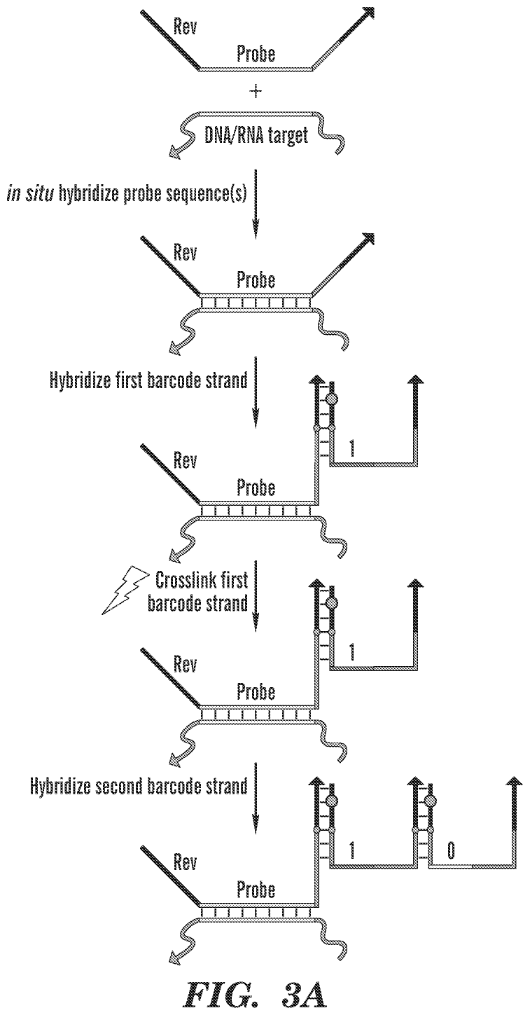

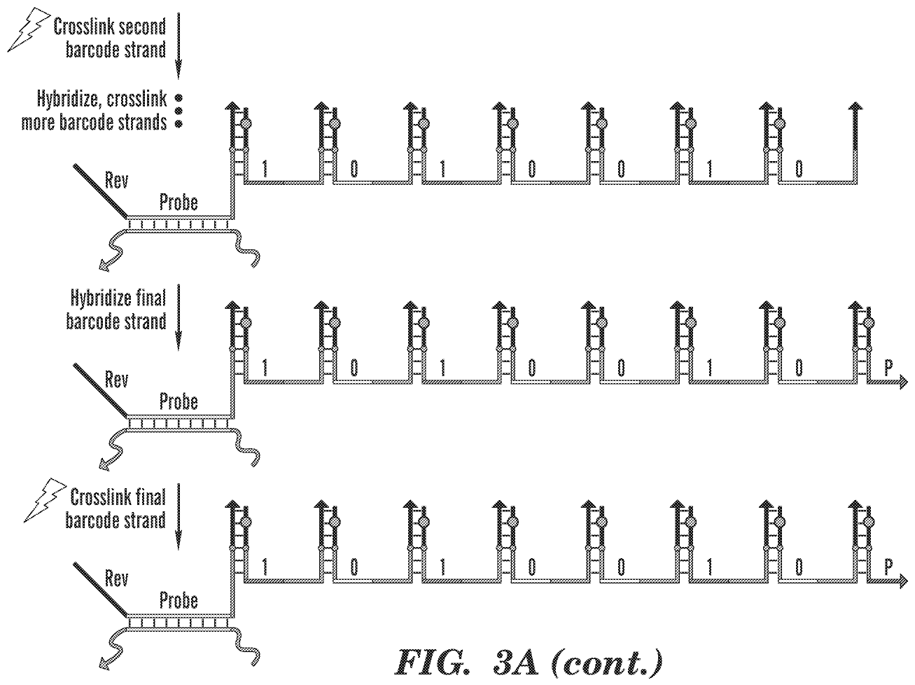

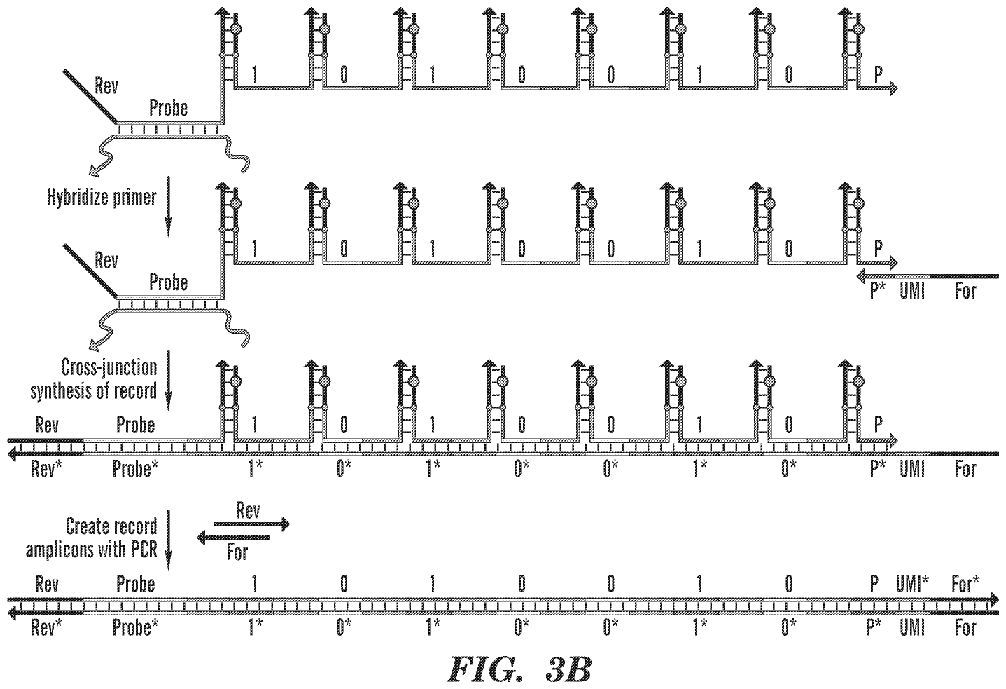

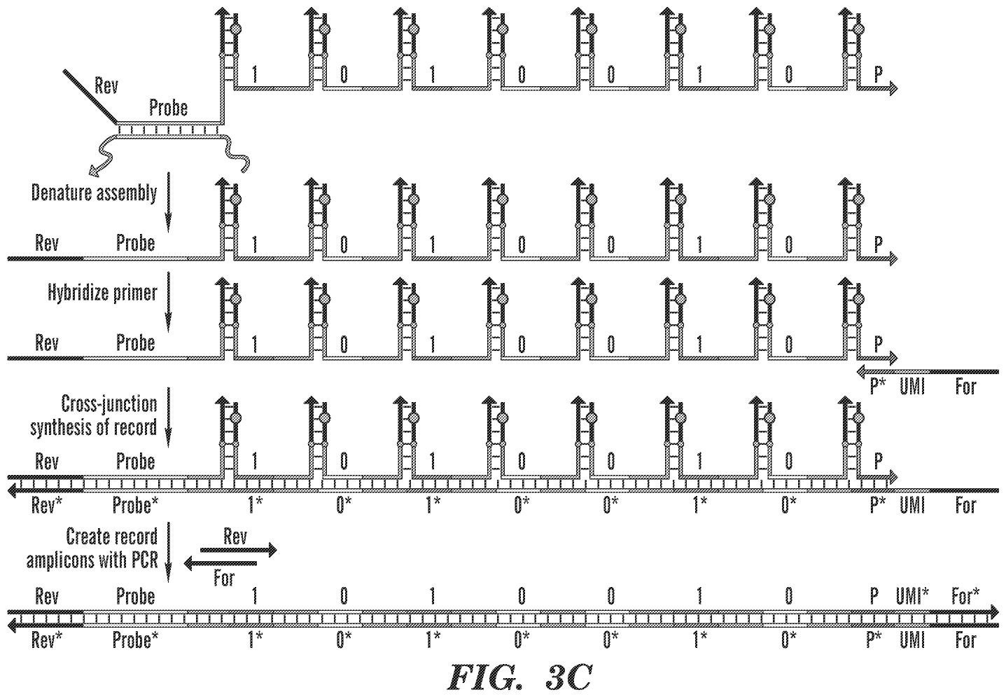

The third strategy again uses only a single wavelength of light (˜365 nm) for crosslinking of CNVK-containing sequences to semi- or fully-complementary sequences. This strategy utilizes multiple rounds of crosslinking are performed on the same regions or sequences, so that a multi-strand complex (concatemer) is assembled, see FIG. 3A-3C. The chain of barcode sequences on a concatemer can then be copied into a sequenceable record strand using cross-junction synthesis.

In a targeted approach, probes designed to be complementary to genomic or transcriptomic sequences of interest are hybridized in situ (FIG. 3A-3C). A secondary hybridization step binds a barcode sequence that contains a CNVK modification in the region complementary to an overhang on the probe on one end of the strand, a barcode sequence domain in the middle of the strand, and a region complementary to another CNVK-containing barcode strand on its other end. Upon illumination under UV light (approximately 365 nm), the first barcode becomes covalently linked (crosslinked) to the probe sequence. A second barcode strand can subsequently be hybridized to the concatemer and crosslinked. Further strands may be crosslinked to iteratively assemble a concatemer sequence. The last concatemer barcode strand (the ‘capping’ barcode strand) bound contains a binding site for a ‘capping’ primer and may or may not be crosslinked to the concatemer assembly.

The final strand introduced is a ‘capping’ primer, which contains a forward primer sequence (For), optionally a unique molecular identifier (UMI), and the primer sequence complementary to the ‘capping’ barcode strand. A strand-displacing polymerase can then be used to copy the full record strand through a cross junction synthesis reaction, which can be done either before (FIG. 3B) or after (FIG. 3C) denaturation from the substrate. Record strands may be PCR amplified with the forward (For) and reverse (Rev) primers before ultimately being sequenced to recover combined barcode/UMI and probe sequence/identity information. The concatemer assembly is depicted on the 3′ overhang of the probe sequence but may also alternatively be performed on the 5′ overhang, so that the cross junction synthesis happens after the probe sequence is copied. This strategy also allows the re-use of the same barcode sequences throughout the concatemer and can be thought of as a combinatorial assembly method.

The targeted approach may also be used to bind other nucleic acids immobilized in a sample or on a surface, such as DNA-conjugated antibodies bound to protein targets of interest (see FIG. 3B). In general, any entity that can be labeled with or crosslinked to a strand of interest can be recorded with this strategy.

Concatemer assembly may also be paired with a non-targeted approach, either by assembling the concatemer on an overhang on the binding domain of a barcode strand (e.g. see FIG. 3A-3C) similar to the methods described in Strategies 1 and 2. The concatemer may also be formed on a 5′ overhang of a template switching oligo (TSO).

Notes on Variations:

Barcode domains may be 0-100 nucleotides in length, or longer and may use 1-, 2-, 3-, or 4-letter code sequences. They may also contain modifications, unnatural, or degenerate bases.

UMI domains may optionally be included in barcode strands and/or probe strands.

UMI domains may be synthesized by using a mix of nucleotides during base addition chemical synthesis to create libraries of random sequences (degenerate sequences). They may consist of several such random bases in tandem, with or without known nucleotide sequences intercalated.

All domains in all strands can be 1-, 2-, 3-, or 4-letter code sequences. They can also comprise modifications, unnatural, or degenerate bases.

The approaches presented can be used to create patterned and barcoded surfaces which can optionally be utilized as oligonucleotide arrays for higher levels of patterning, masking, and capturing.

The targeted approach may also be used to bind other nucleic acids immobilized in a sample or on a surface, such as DNA-conjugated antibodies bound to protein targets of interest (see FIG. 1B). In general, any entity (such as nucleic acids, proteins, peptides, lipids, sugar groups, small molecules, nanoparticles, beads, glass surfaces) that can be labeled with or crosslinked to a strand of interest can be patterned, barcoded and recorded with this strategy.

Crosslinking reversal (Strategy 1) may be performed before or after record synthesis with a polymerase.

Crosslinking reversal (Strategy 1) can be performed under chaotropic or denaturing conditions such as in urea, guanidinium chloride, or formamide-containing buffers or under low salt conditions.

Crosslinking reversal (Strategy 1) can be performed under high temperature conditions.

Crosslinking reversal (Strategy 1) may be performed in the presence of strand displacing polymerase.

The barcode domain may be 5′ or 3′ of the binding domain (e.g. the domain binding a polyA tail of an mRNA) for Strategy 2.

In the concatemer assembly approach (Strategy 3), an arbitrary number of rounds can be used to produce arbitrary length concatemers (e.g. comprising 1, 2, 3, or up to 500 strands or more).

In the concatemer assembly approach, anywhere from 2 to 100 or more distinct barcode sequences per round.

PCR can be performed before sequencing of records. Records may also be further processed to prepare for next-generation sequencing.

UMIs can optionally be excluded from primers and record sequences.

Barcode strands can comprise a modification on the 3′ end to inhibit extension by polymerase. They may alternatively contain a ‘tail’, such as a series of T bases to prevent extension. They may also not be prevented from extension by a polymerase.

In some variations, the primers on either side of an amplicon (e.g. For and Rev domains) may be identical.

An alternative to crosslinking utilizing a CNVK base is to use a photocleavable spacer on the 5′ end of a barcode strand that allows ligation of the barcode strand to the 3′ end of a probe or other sequence. Strands that are not cleaved would not be covalently linked to the probe/target and could be washed away before subsequent barcoding rounds.

Crosslinking can be performed at UV (300-400 nm) or near UV wavelengths (400-500 nm), or at higher wavelengths by using 2-photon illumination.

Wavelengths for reversal of crosslinking can be performed at UV and near UV wavelengths (300-405 nm).

Up-converting nanoparticles can be utilized to perform crosslinking at different wavelengths.

Other methods can be used to convert crosslinked assemblies to sequenceable records. For example, a restriction site may be used within the binding regions between bound barcode strands, and a hairpin may be ligated to cleaved ends to form a complete record strand. Alternatively strands that bridge across junctions may be bound to the assembly and then ligated together, possibly after or during a gap-filling step with a polymerase.

Other methods can be used to observe or validate the barcoding process such as use of fluorophores or nanoparticles for microscopic observation.

An alternative to directly assembling barcodes on biomolecules of interest, the barcodes can be formed on molecules nearby, such as on strands that are covalently linked to a hydrogel matrix. These nearby assemblies may then be converted to records by either reaching across to other molecules and copying sequence information, or through ligation or otherwise physical linking of proximal sequences (e.g. with strategies from Hi-C or DNA microscopy).

With the targeted approach, the reverse primer site (Rev) may instead be moved to the other overhang strand (on the 3′ end of the probe sequence) with a probe-identifying domain 3′ between the Rev domain and the domain that binds barcode strands. This probe-identifying domain may be 0, 1, 2, up to 50 or more bases in length and could serve as an index to identify what probe sequence was bound without actually requiring the probe binding sequence itself to be sequenced.

Barcoded biomolecules are also compatible with downstream assays. For example, proteins might be non-specifically labeled (conjugated to) a nucleic acid strand which is subsequently barcoded. After barcoding, the proteins may be purified from a sample and applied to a protein or antibody micro-array to reveal the identity of the protein, which can also be barcoded onto the target (e.g. by assembling a larger barcode concatemer). In general, any downstream assay that physically separates or sorts the molecules in some way (e.g. gels, western blots, FACS, size exclusion columns) can utilize subsequent barcoding steps to encode additional information about the target/transcript in the assembled barcode sequence.

Secondary assays can follow the barcoding for further analyses. These may include qPCR, microscopy, pull-downs, DNA/RNA microarrays, protein microarrays, antibody arrays, electrophoresis gels, western blots, cell sorting, FACS, Droplet or microfluidic based methods, mass spectrometry, mass spectrometry imaging, laser microdissection.

### Example 3: Spatial Patterning with Iterative Light Crosslinking

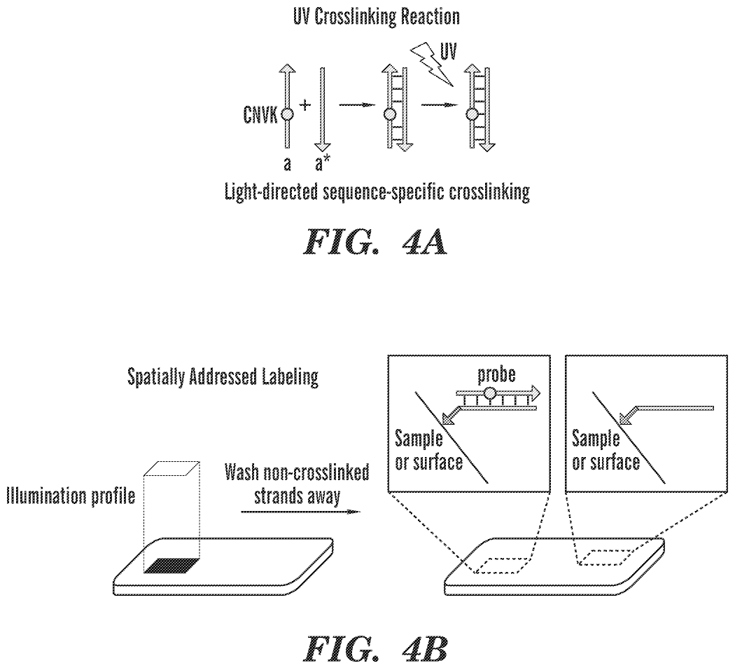

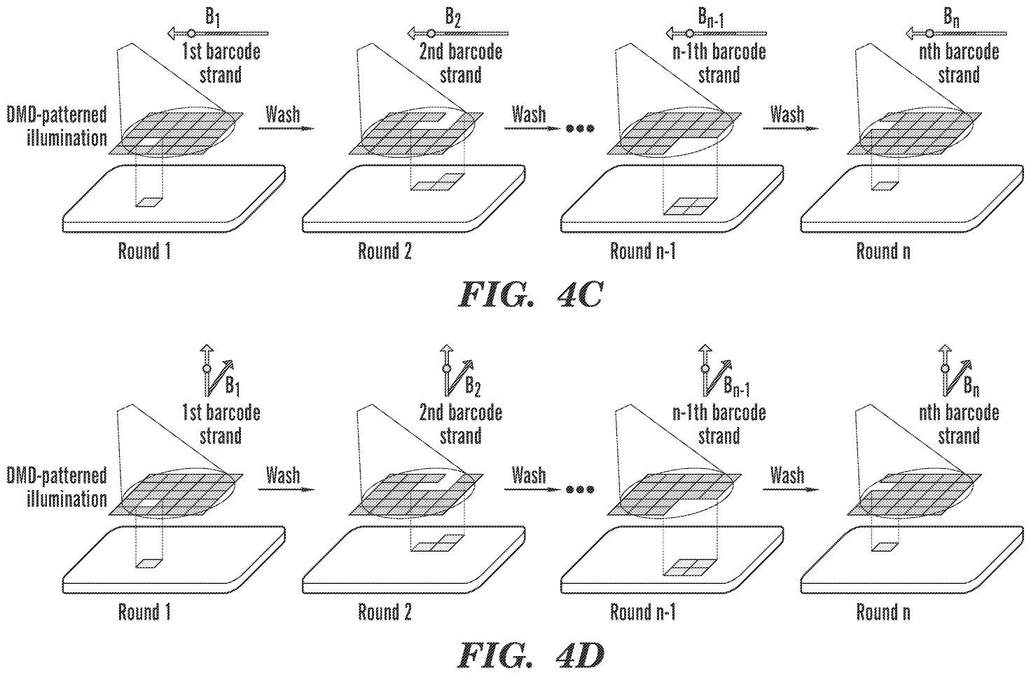

Any light-directed barcoding strategy (e.g. Strategies 1-3 above) may be paired with iterative rounds of spatially patterned illumination to achieve higher levels of multiplexed sequencing readouts. The basic crosslinking reaction is depicted in FIG. 4A. A sequence containing a CNVK modification binds to a partially or fully complementary sequence, and a covalent bond is formed upon UV illumination. By spatially confining the area or volume of light illumination to a specific region or set of regions, the crosslinking can be made to occur only within the illuminated area(s) (FIG. 4B). After washing away non-crosslinked strands, only the region(s) will remain bound to the crosslinked strand.

Distinct barcode sequences are assembled at different positions in situ by utilizing iterative rounds of hybridization and crosslinking using the chosen light-directed barcoding strategy and can be pooled together in the same sequencing run following the barcoding procedures described in the previous section. Upon sequencing, barcode sequences are used to map the sequencing data to the original specified (illuminated) position(s) during the barcoding round associated with the barcode sequence. This sequencing data may optionally be further paired with microscopy or other types of analysis of the sample or surface of interest to provide even higher dimensional data. Figures below are shown for patterned illumination utilizing a Digital Micromirror Device (DMD), but any device capable of programmable light illumination (such as Point Scanning Confocals, Spinning Disk Confocals, Light Sheet Microscopes, High Throughput Scanners, Structured Illumination Microscopes, Stimulated Emission Depletion Microscopes) can be combined with the barcoding chemistries.

In some experiments, multiple regions may receive the same barcode sequence(s) during the same round, which may represent a property other than spatial positioning. For example, if all cells with the same marker gene or other shared property (e.g. same cell state) are labeled with the same barcode sequence, then their sequencing reads can later be grouped together. In some experiments illumination may be done at a sub-cellular level, on just the nucleus region, at the whole cell level, or at a level larger than a cell. Illumination may be performed in fixed cell or tissue samples, or also directly onto a functionalized surface.

Approach: Spatial patterning with iterative light crosslinking using dual wavelengths (Strategy 1). An example of iterative light crosslinking enabling multiple (n) regions to be labeled with unique barcode sequences (B1 through Bn) utilizing the first strategy described for light-directed barcoding is depicted in FIG. 4C. Each round would consist of a hybridization step where barcode strands are bound to all regions, a crosslinking step where illumination is confined to a specific programmed region (or regions), and a wash step that dissociates all non-crosslinked barcode strands from the sample/substrate. Optionally, the crosslinking can also be performed during the hybridization step. Specified regions can each receive a barcode strand with a unique barcode sequence (B1 through Bn), which is later recovered during sequencing to allow the probe/transcript sequence information to be mapped back to the illuminated region(s).

Approach: Spatial patterning with iterative light crosslinking using bridge sequences (Strategy 2). An example of iterative light crosslinking enabling multiple (n) regions to be labeled with unique barcode sequences (B1 through Bn) utilizing the second strategy described for light-directed barcoding is depicted in FIG. 4D. Each round would consist of a hybridization step where barcode strands are bound to all regions, a crosslinking step where illumination is confined to a specific programmed region (or regions), and a wash step that dissociates all non-crosslinked barcode strands from the sample/substrate. Optionally, the crosslinking can also be performed during the hybridization step. Specified regions can each receive a barcode strand with a unique barcode sequence (B1 through Bn), which is later recovered during sequencing to allow the probe/transcript sequence information to be mapped back to the illuminated region(s).

Approach: Spatial Patterning with Iterative Light Crosslinking and Concatemer Assembly to Create Combinatorial Barcodes (Strategy 3).

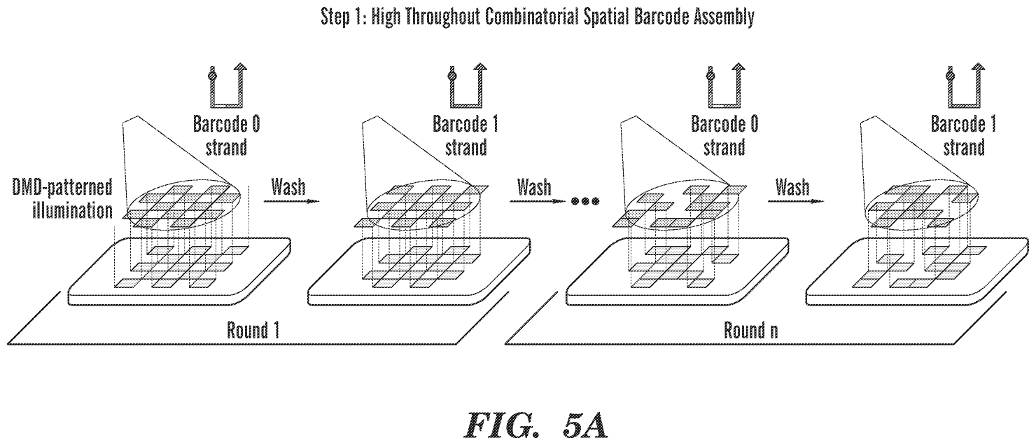

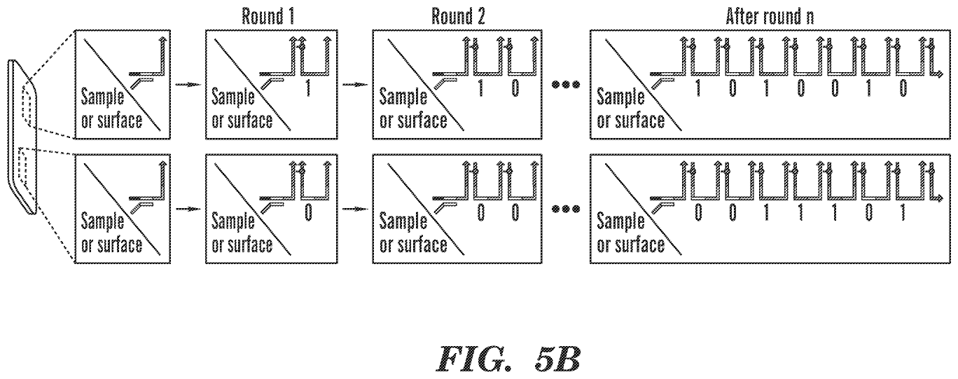

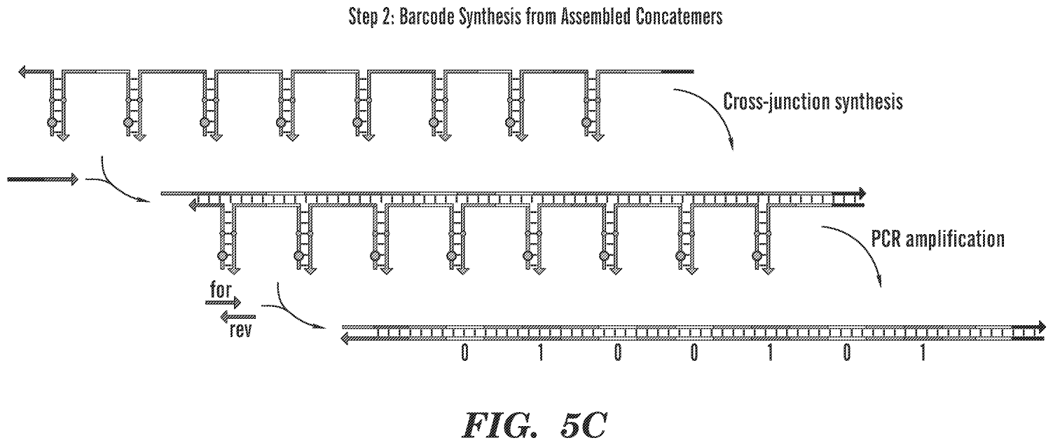

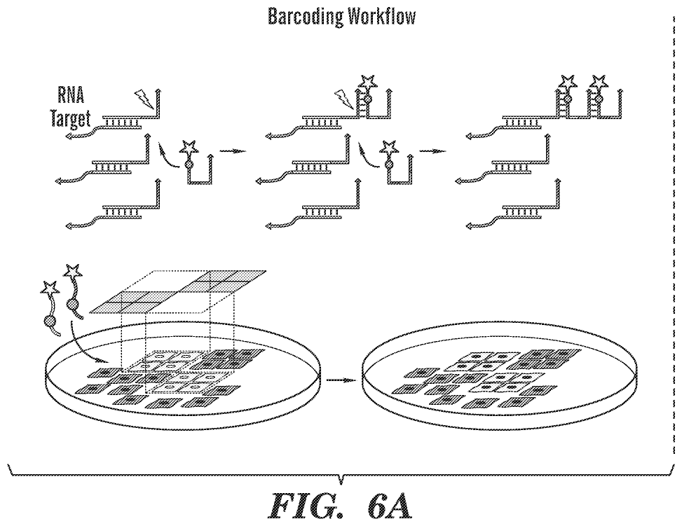

The strategy for massively-multiplexed barcode is depicted in FIG. 5A-5C. The strategy is divided into two parts. In the first phase DNA barcodes are iteratively photo-crosslinked to a growing strand in a unique crosslinking geometry which will serve as a template for enzymatic copying in the second phase (FIG. 5A). The second phase utilizes a strand-displacing DNA polymerase to copy across the assembled chain of crosslinked barcodes to copy the barcode information into a single contiguous DNA strand, the information of which can then be retrieved through sequencing (FIG. 5B).

The following steps would take place for each barcode strand in each round: a hybridization step where barcode strands are bound to all regions, a crosslinking step where illumination is confined to a specific programmed region (or regions), and a wash step that dissociates all non-crosslinked barcode strands from the sample/substrate. Optionally, the crosslinking can also be performed during the hybridization step. Each round consists of multiple barcode strands undergoing this process. If m barcode strands are used in each of n rounds to construct concatemers containing n barcode sequences, for example, then there are m″n possible concatemer sequences that can be programmatically assembled. In FIG. 5A, an example of m=2 is shown, so there would be 2″n possible programmable concatemer sequences in n rounds.

Experimental Validation

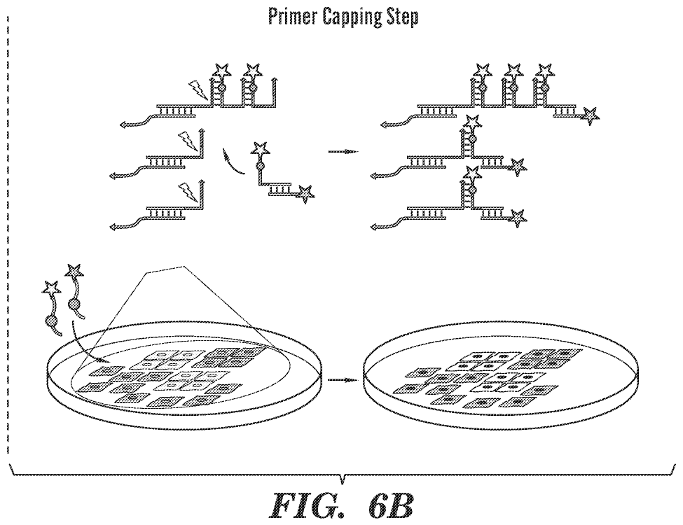

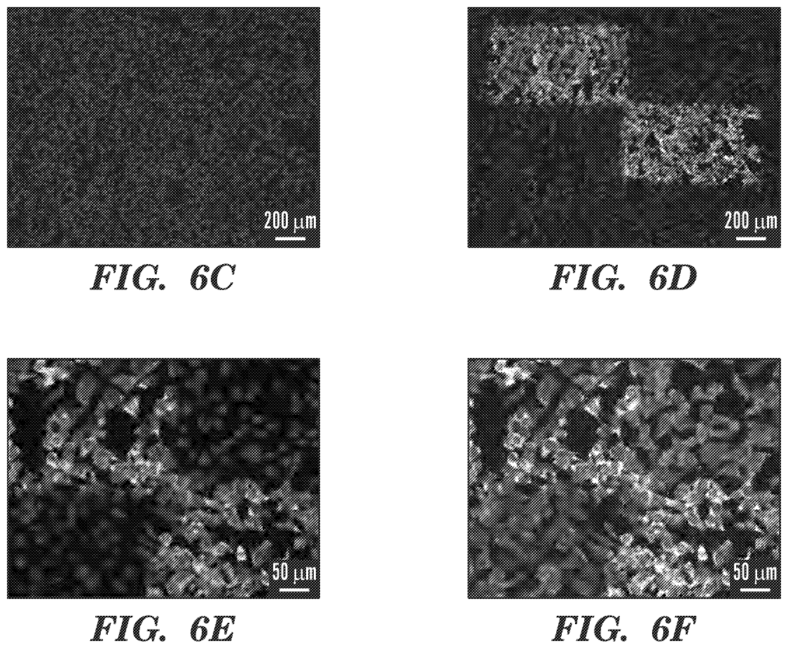

Spatially patterned illumination was validated on fixed EY.T4 cells. Cells were fixed as a monolayer using 4% PFA to well chambers on a coverslip. Subsequently, several washes as well as a 10 minute incubation in 1×PBS with 0.5% (vol/vol) Triton X-100 to permeabilize the cells were performed, and a probe targeting ribosomal RNA (rRNA) was hybridized in situ overnight at 37 C in a buffer comprising 2×SSCT, 50% formamide, 10% dextran, 0.1% Tween-20, and ˜67 nM probe sequence after a 3 minute incubation at 60 C following standard protocols. The probe sequence contained a 3′ overhang to which the first barcode strand could bind. For validation the barcode strand carried a Cy3b fluorophore on the 5′end. Cell samples were incubated for 10 min with 50 nM of the first barcode strand in PBS. Unbound strands were washed with PBS for 3×1 min. A chosen area was then exposed to a 365 nm UV laser (5 with a power density of 10 w/cm″2 out of the fiber) for 2 sec to induce crosslinking using a DMD with a 4× objective. Uncrosslinked strands were washed with 50% formamide in PBS for 2×2.5 min. After a 1 min wash with PBS, nuclei were labeled with DAPI and imaged at 20× with a wide-field microscope (FIG. 6A-6F).

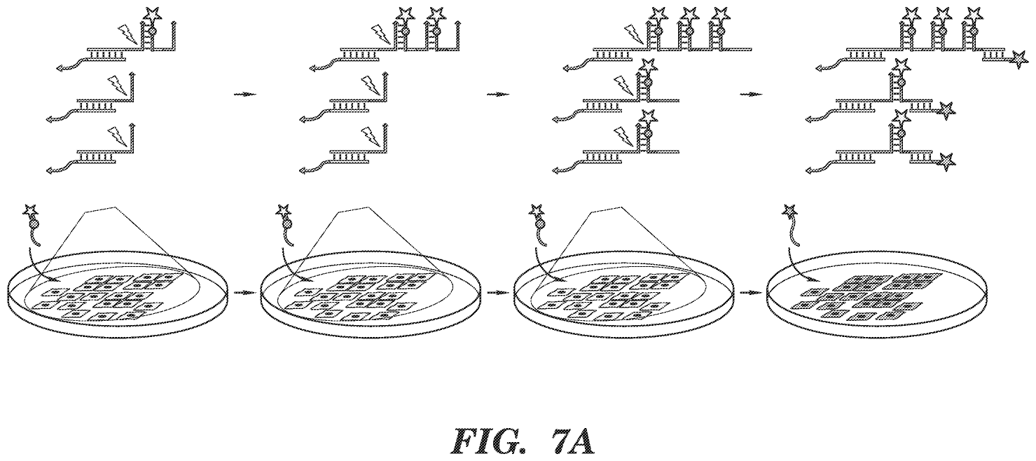

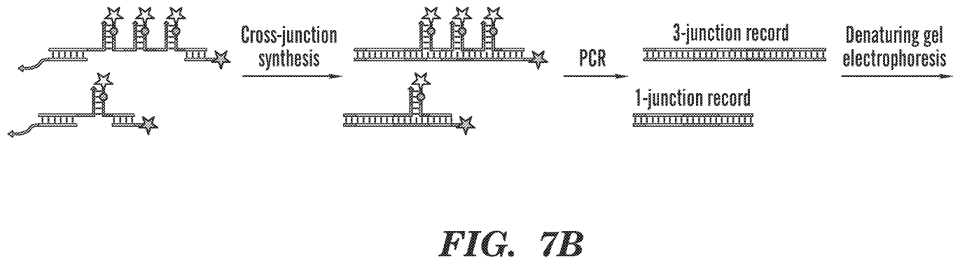

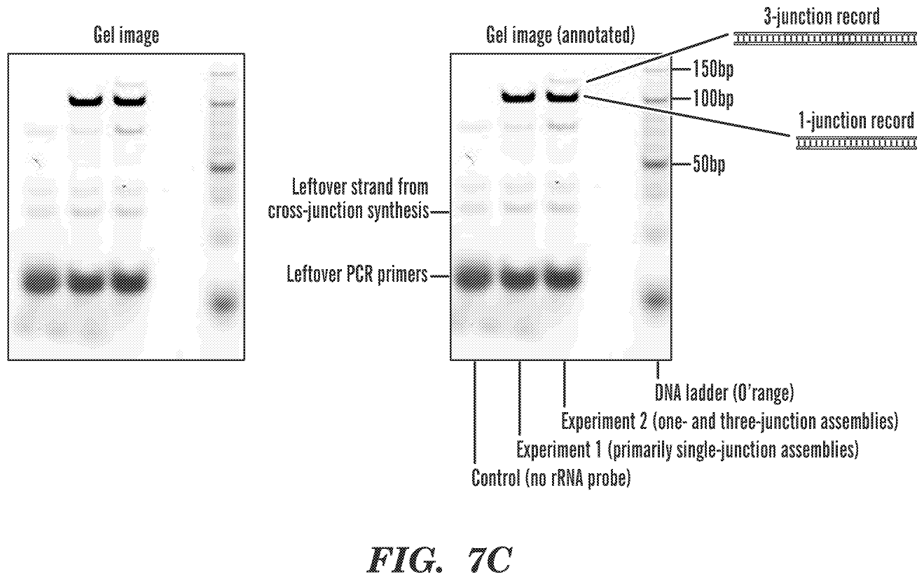

Iterative crosslinking for biomolecular barcoding was also tested using the same type of rRNA-targeting sample. In this instance, the entire sample was illuminated at each step with a hand held UV gun that outputs light at 365 nm with a power density of 2 w/cm″2, and concatemers containing up to three barcode strands were assembled sequentially. In each round 50 nM of Cy3b-labeled barcode strands were applied onto cells for 10 min in PBS, followed by removal of unbound strands by for 3×1 min PBS washes, UV exposure, and removal of uncrosslinked strands with 2×3 min washes with 50% formamide in PBS. At the final round the Cy5-labeled primer strand (primer capping) that was applied and used for cross junction DNA synthesis (FIG. 7A). After cross junction synthesis and PCR amplification, strands of the correct length for one- and three junction assemblies were visualized on a 15% TBE-Urea PAGE denaturing gel (FIG. 7B, Experiment 2).

Another sample with primarily single junction assemblies (corresponding to the sample in FIG. 6A-6F, which contained only small regions patterned to the longer assemblies in the whole sample) was also visualized after cross junction synthesis and PCR (Experiment 1). Finally, a control sample with no underlying probe (no probe during in situ hybridization), but receiving all subsequent barcode and crosslinking treatments, was run through the protocol and did not produce strands of record lengths as expected.

### Example 4: Validation of Spatial Labeling and Sequencing

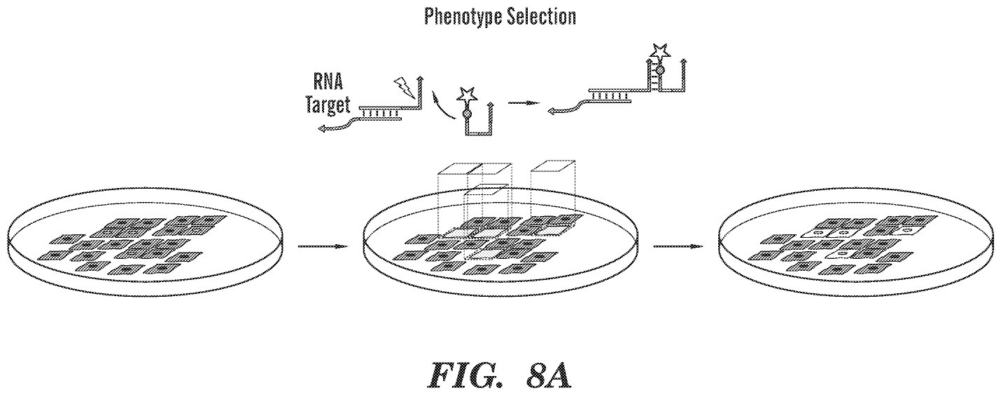

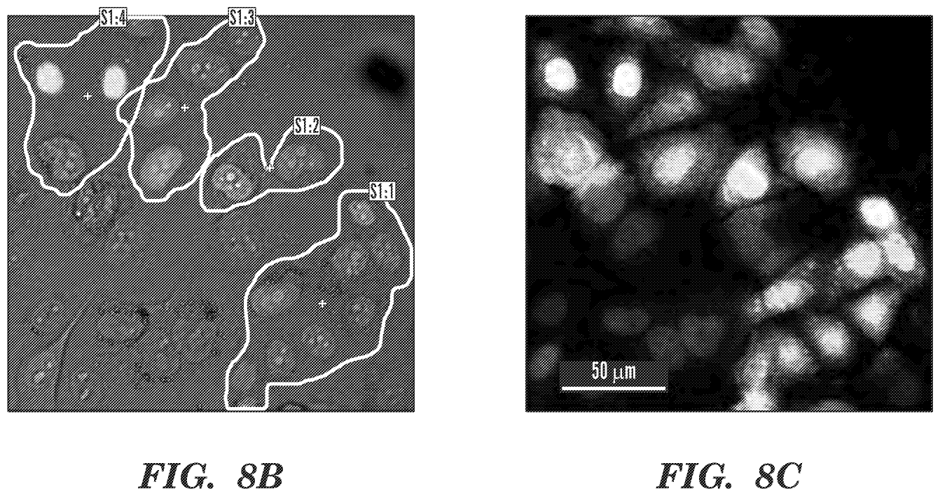

FIG. 8A-8C shows experimental validation of cell-level spatial labeling. Multiple regions of interest (yellow, blue, green, red outlines) pre-selected for crosslinking are drawn around the cells displaying GFP signal (FIG. 8B).

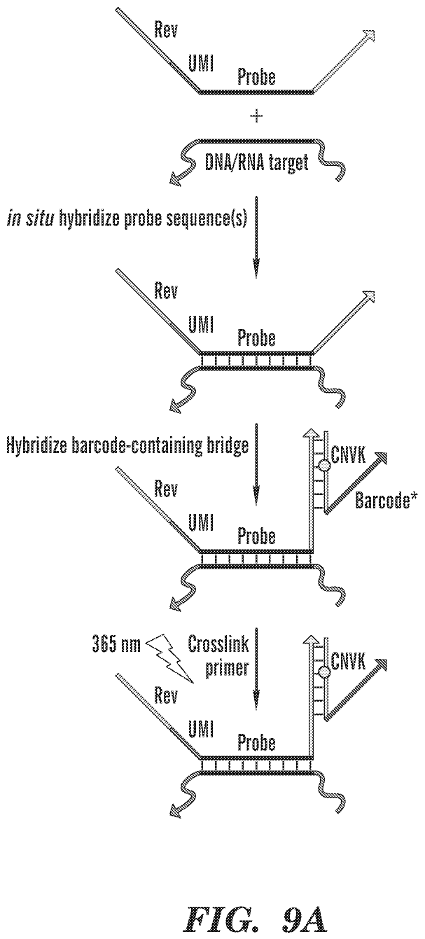

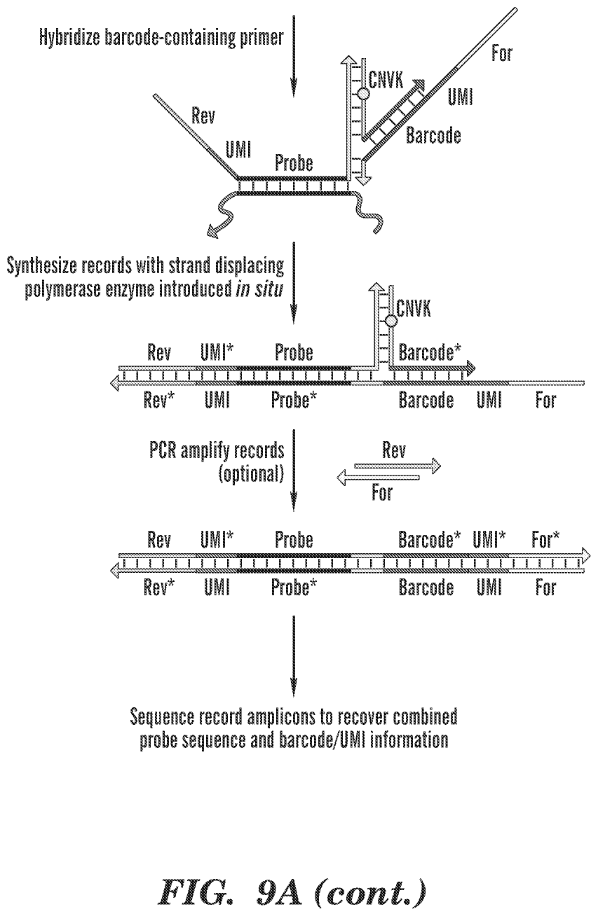

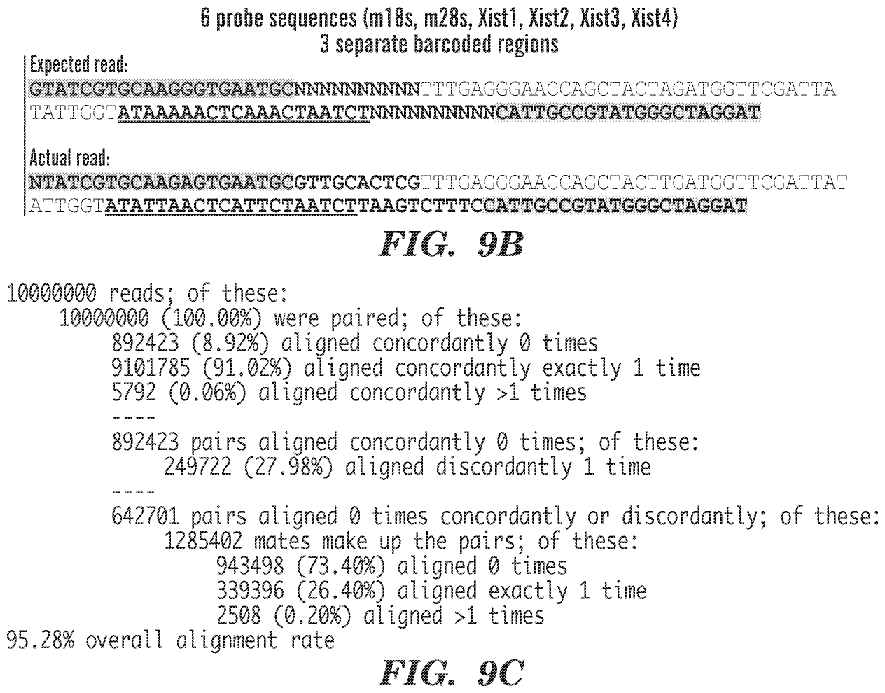

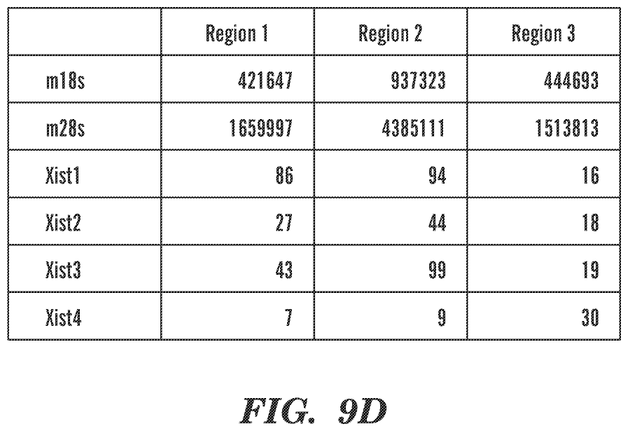

FIG. 9A-9D shows sequencing results. Utilizing a variant of Strategy 2, with UMIs on both ends of the amplicon, three distinct spatially separated regions were serially barcoded using patterned illumination on fixed HeLa cells. FIG. 9A demonstrates that 6 distinct probe sequences (two targeting ribosomal RNA and four targeting the Xist RNA) were bound to their target RNA sequences with FISH. This was followed by iterative barcoding, binding of barcode-containing primers, synthesis, and amplification of records. Amplicons were prepared for Next Generation Sequencing (HiSeq) using a Collibri sequencing prep kit. FIG. 9B-9C show reads of the anticipated format were recovered with high percentage following alignment. FIG. 9D shows read distributions for a large subset of the data are shown for each probe-region pair.

### Example 5: Barcoding Methods

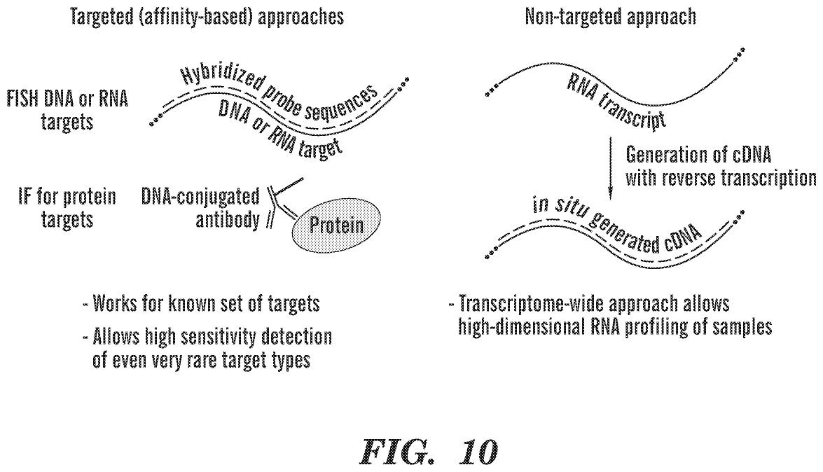

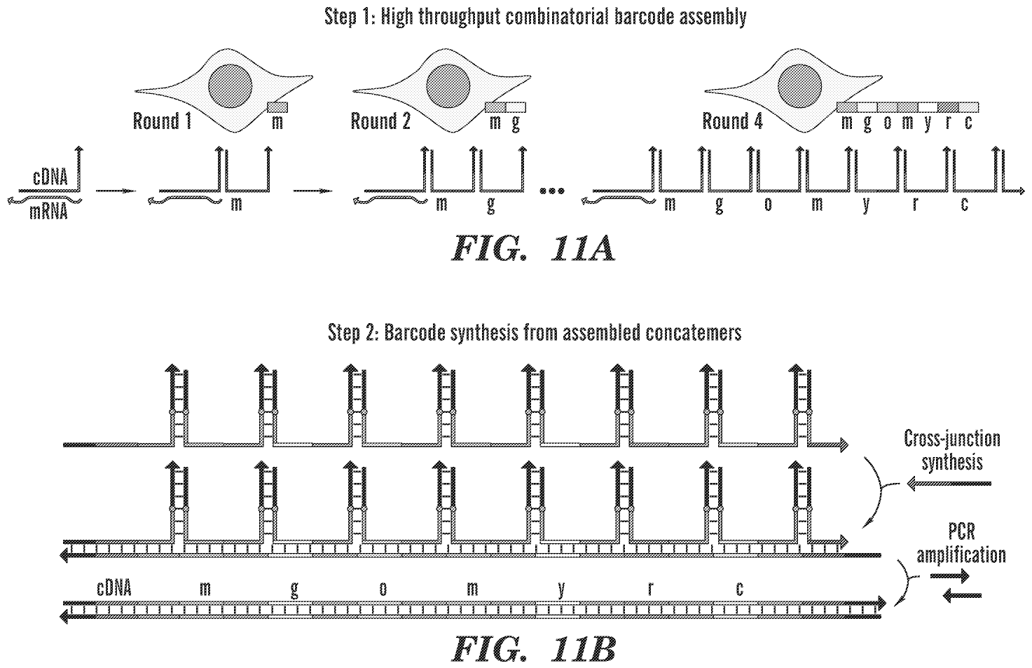

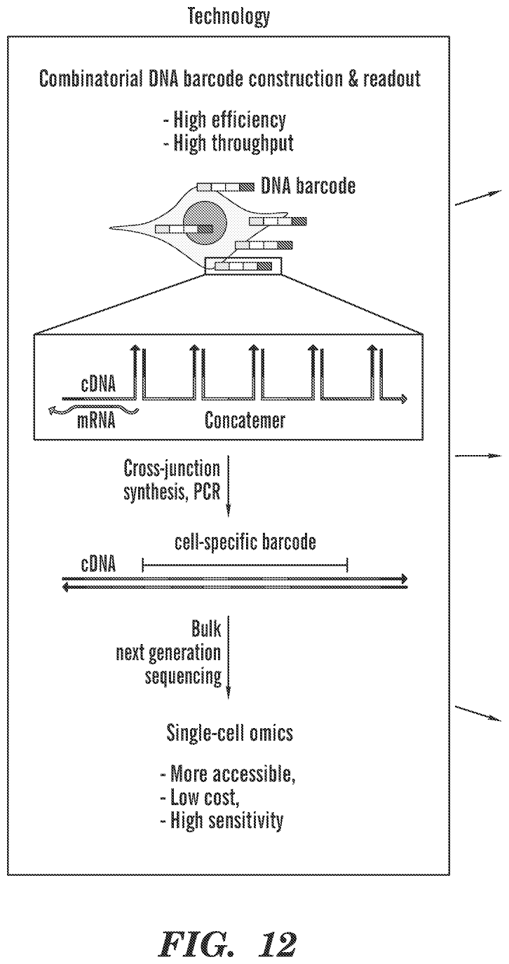

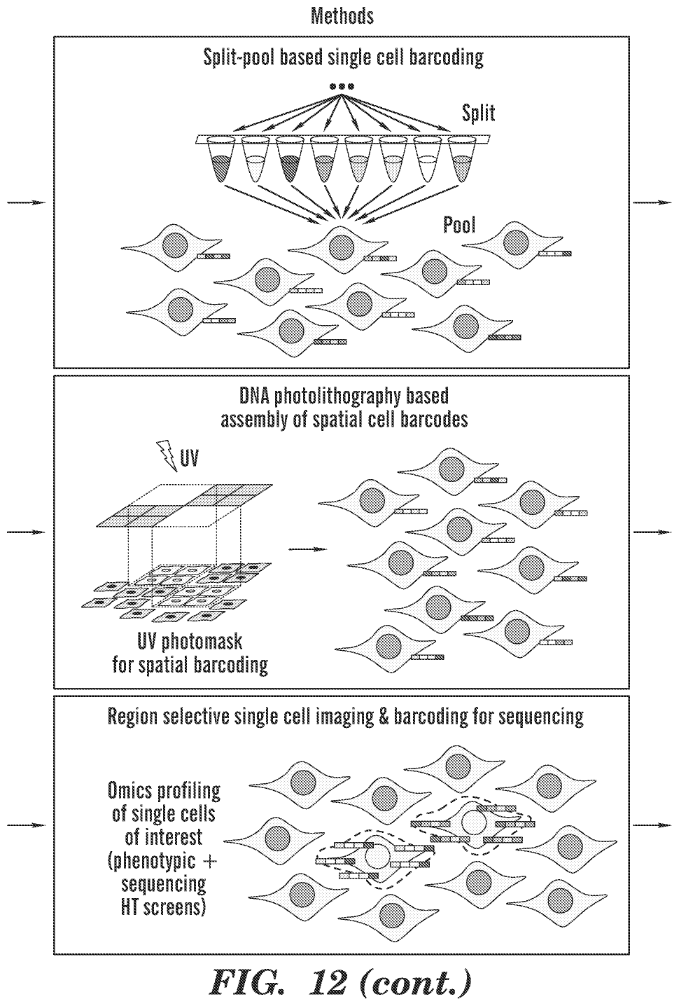

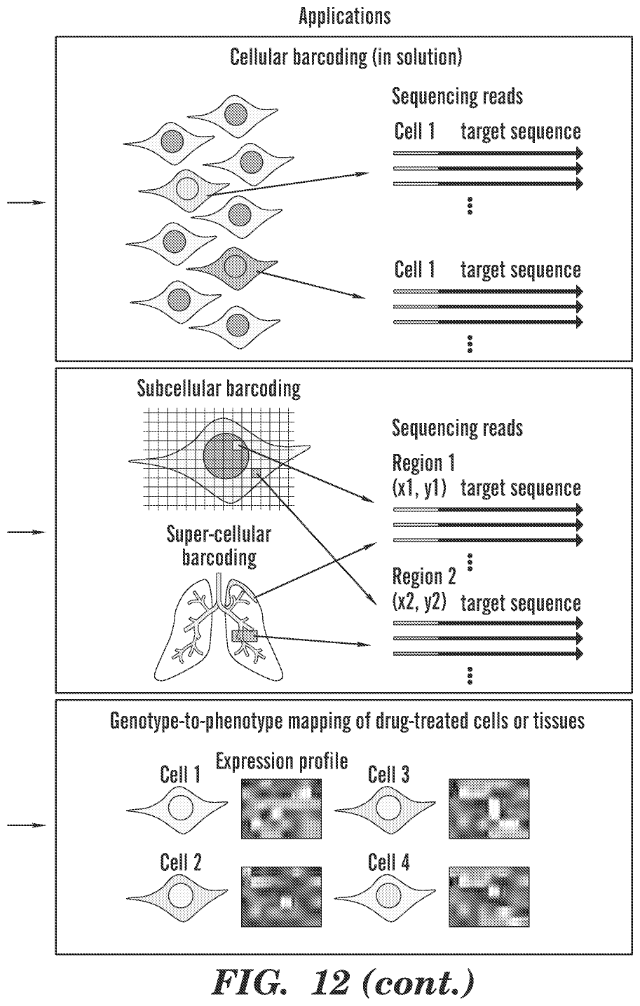

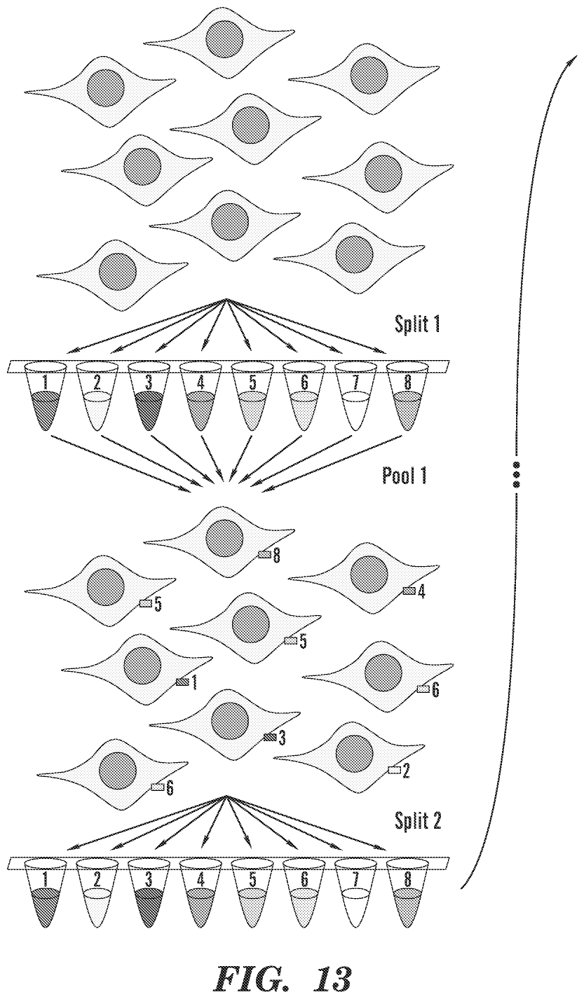

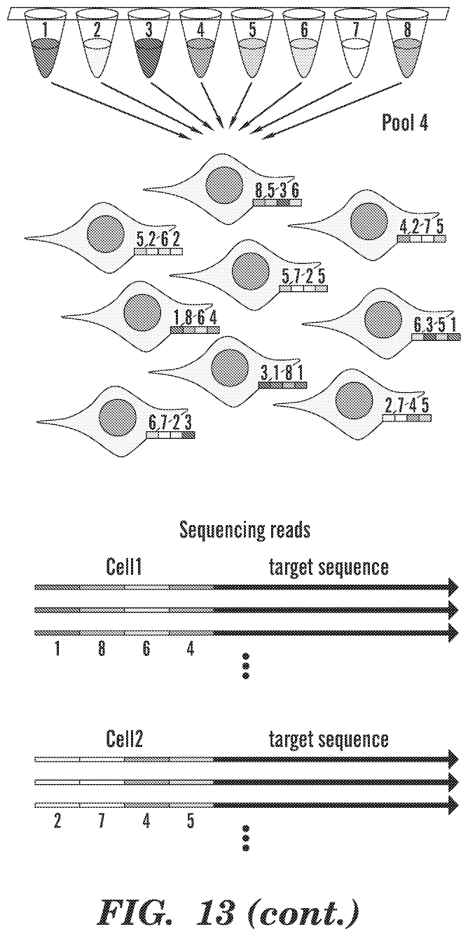

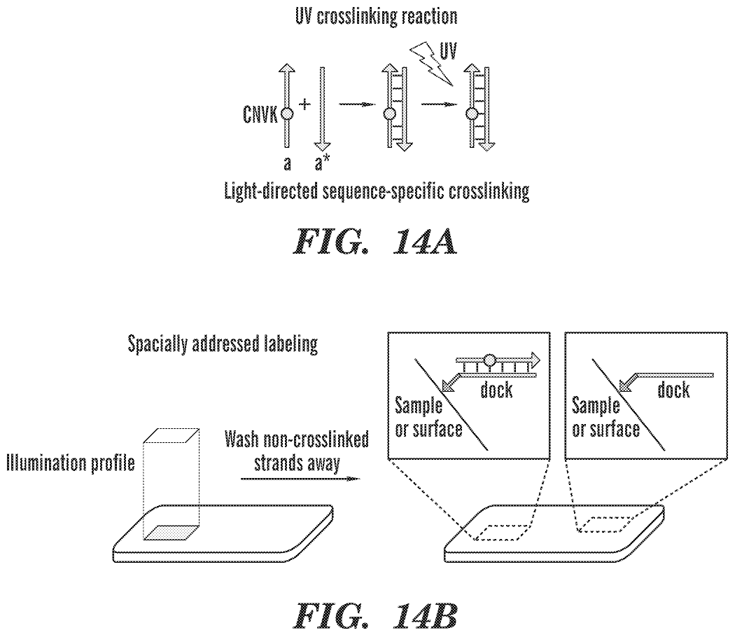

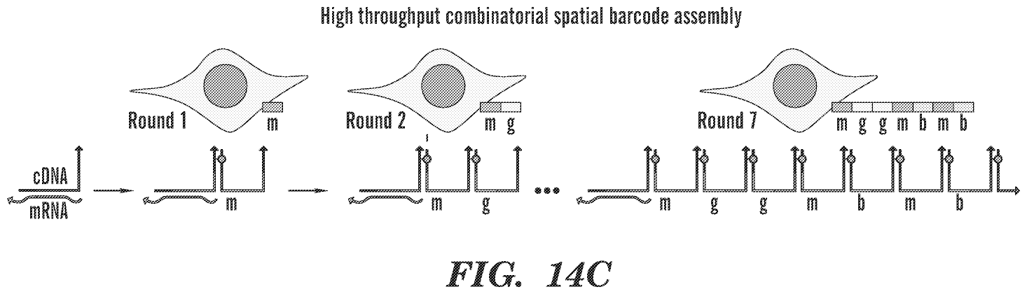

Targeted barcoding can be performed on cDNA sequences, FISH probe sequences, nucleic acids conjugated to antibodies, or any other nucleic acids localized in situ to biomolecules of interest via affinity reagents. Alternatively, non-targeted approaches such as the generation of cDNA sequences using random primers for transcriptome-wide profiling, may act as substrates for barcoding that can be performed on any pre-existing RNA or DNA sequences or other nucleic acid polymers with modified backbones such as LNA or PNA or nucleic acid analogues or modified monomers, or other reaction products in situ generated by the action of polymerases, ligases, restriction enzymes, nucleases, telomerases, terminal transferases, recombinases or transposases such as those of proximity ligation assay, primer exchange reaction, autocyclic proximity recording, or tagmentation (FIG. 10). Barcoding can be performed iteratively to form known permutations of barcodes arranged in multi junction concatemers for reads extracted from specific regions (e.g. a single cell, FIG. 11A). Cross junction synthesis and PCR can be used to extract sequenceable reads from these concatemers. This type of in situ combinatorial barcode construction on biomolecules has a number of possible applications, including single cell split-pool barcoding (FIG. 13), assembly of spatial barcodes on individual cells or sub- and super-cellular regions of interest (FIG. 14), and specific barcoding of cells with certain phenotyping e.g. for drug discovery (FIG. 12).

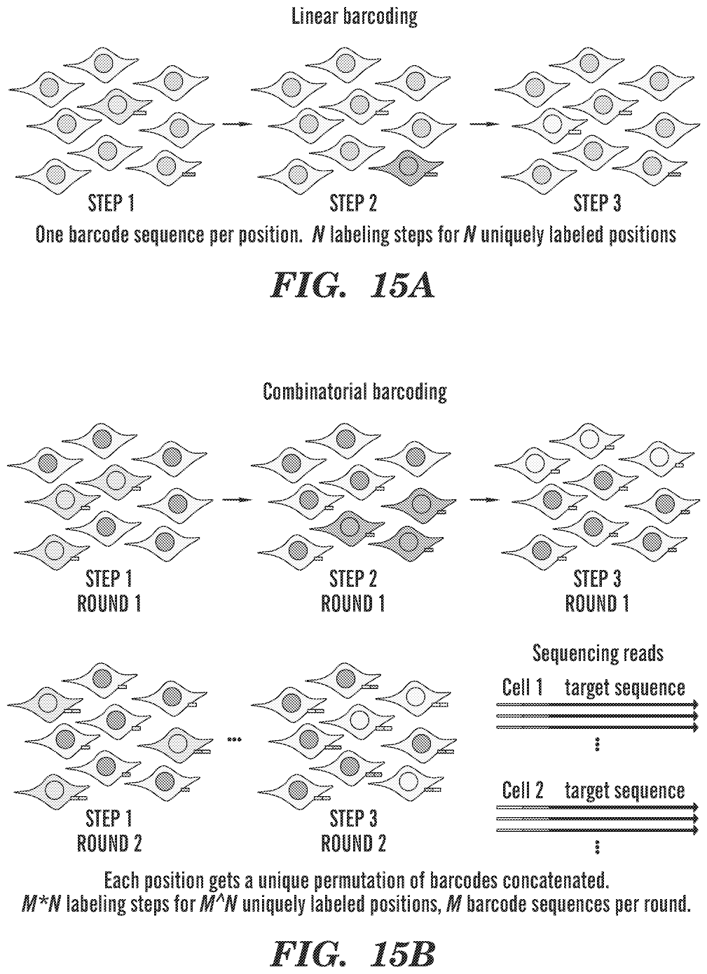

Barcoding may be performed in a linear fashion, where each barcoded region receives a single unique barcode (FIG. 15A). Alternatively, junction concatemers may be formed in a combinatorial manner, whereby N rounds with M barcodes each can generate MAN unique barcode permutations (FIG. 15B).

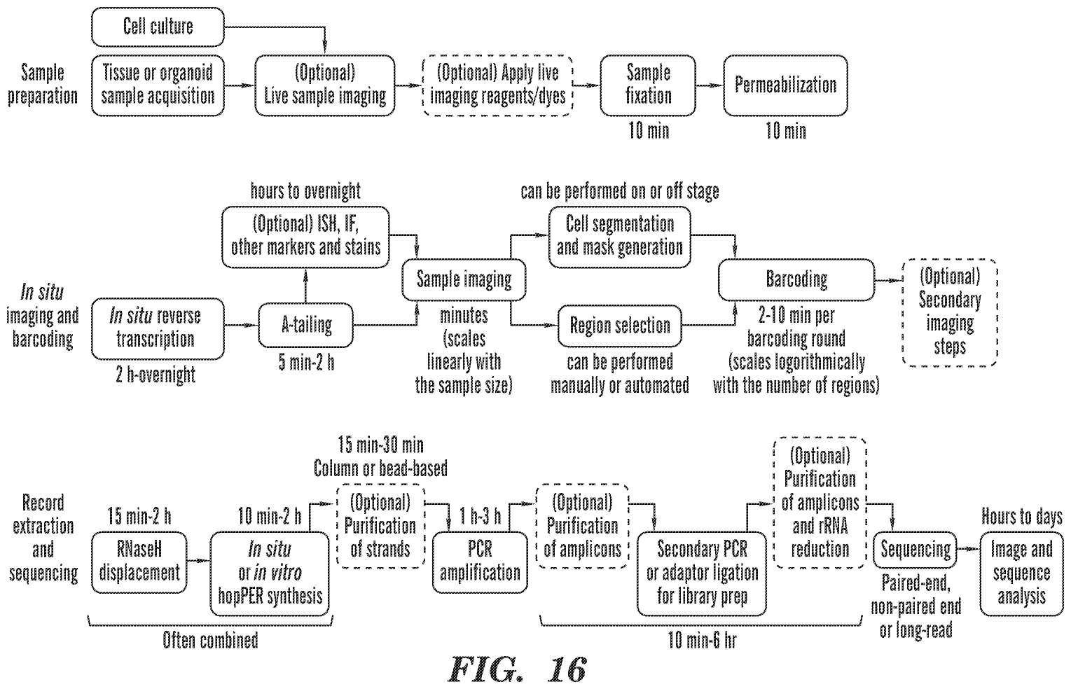

In general, the barcoding can be used to link morphological imaging based datasets directly with sequencing datasets associated with the exact same samples or regions of interest. The general workflow for combining RNA sequencing with imaging data is described in FIG. 16. Cells, tissues, or organoids can all be barcoded after fixation and permeabilization. For transcriptomic analysis, in situ reverse transcribed cDNA sequences and/or FISH-based probes may be substrates for barcoding. For proteomics and other types of -omics analysis, nucleic acids conjugated to antibodies, proteins, nanobodies, or other affinity reagents may act as targets or barcoding substrates. In some cases a tailing step (e.g. “A-tailing”) may be required to add a 3′ overhang to the nucleic acid being barcoded (e.g. a cDNA sequence). After the desired imaging assays are performed, cells and/or sub- or super-cellular regions are then barcoded through iterative construction of junction concatemers. Concatemers bound to RNA may be displaced using an enzyme that specifically cleaves RNA (e.g. RNaseH), which may optionally happen with the subsequent synthesis step. Cross junction synthesis may be performed directly in situ, or during/after displacement (if applicable). After PCR amplification of complete records, amplicons are prepared for sequencing (e.g. purified, analyzed through gel electrophoresis, library preparation) and then sequenced. Barcodes are extracted from sequencing reads in order to map those sequencing reads back to the specific regions that were assigned those barcodes.

Tailing (e.g. “A-tailing”) may be achieved through the use of a terminal transferase enzyme and dATP. ddATP or another terminating nucleotide may optionally be included at a low concentration to randomly terminate the 3′ end so that it is protected from subsequent extension during the cross junction synthesis step. Tailing may instead be performed with a different nucleotide, e.g. dCTP, dGTP, or dTTP, or a mix of nucleotides. Other strategies may also be used to add a 3′ overhang, e.g., ligation.

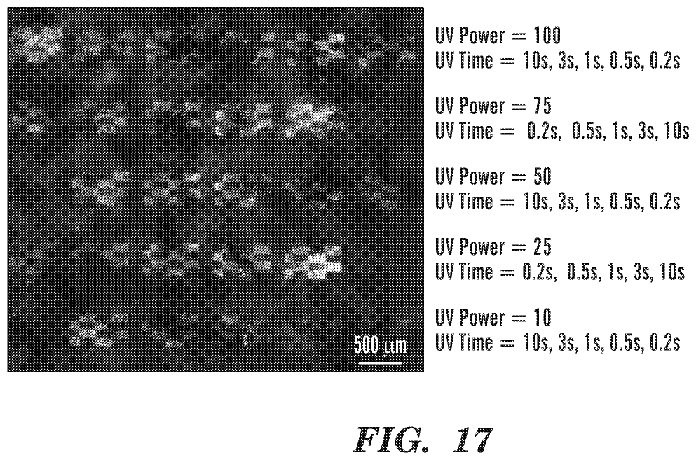

Different UV power and illumination time conditions were tested on prepared HeLa cells. A FISH probe targeting rRNA was hybridized in situ and acted as a barcoding substrate via its 5′ overhanging domain (FIG. 17). A control macro was created to automatically raster the sample across multiple fields of view, illuminate an area with a checkerboard pattern, and adjust the UV power and illumination time accordingly. An optimal UV condition for each particular light source maximizes crosslinking efficiency and minimizes off-target crosslinking. Depending on the light source, wavelength, power, distance, magnification, focus, and other constraints, this amount of illumination time may vary widely, e.g. between 1 ms and several minutes or more. For example, this illumination might be 1 ms, 5 ms, 10 ms, 100 ms, 1000 ms, 10000 ms, 100000 ms, 1000000 ms, etc. with a power of 1%, 2%, 5%, 10%, 100%, etc.

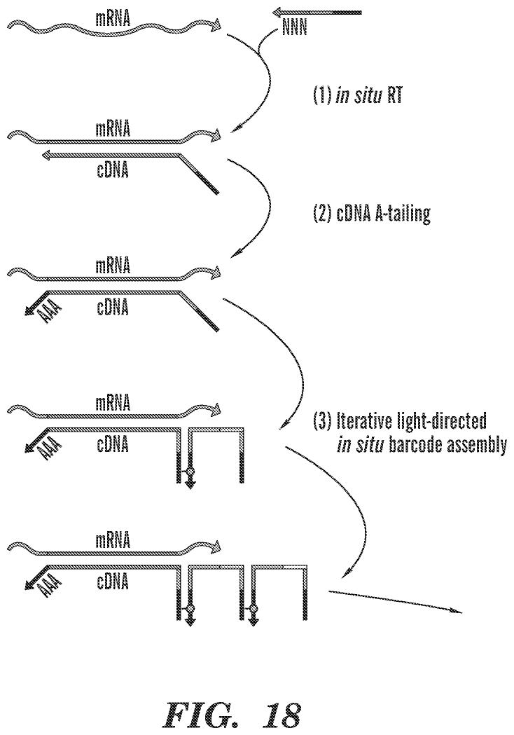

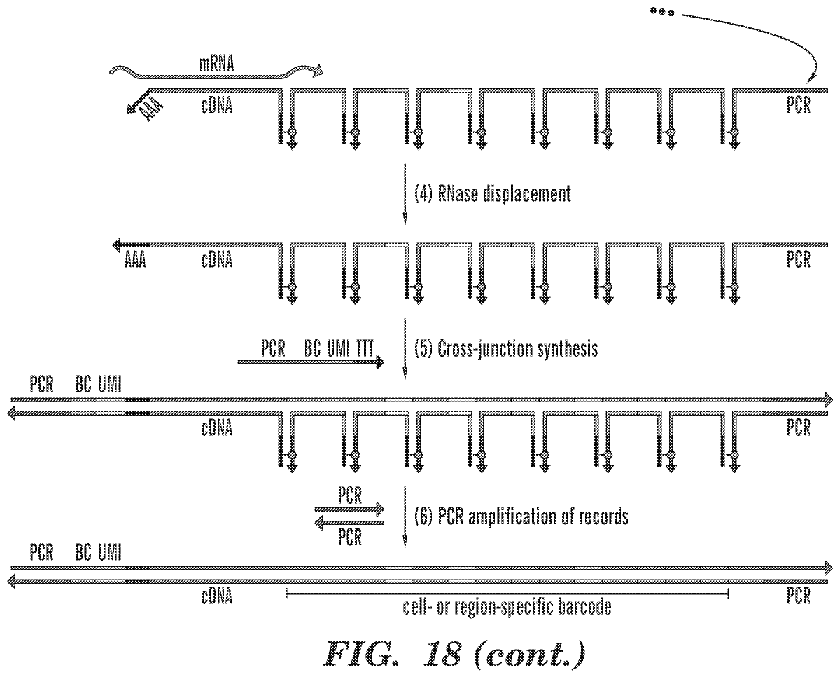

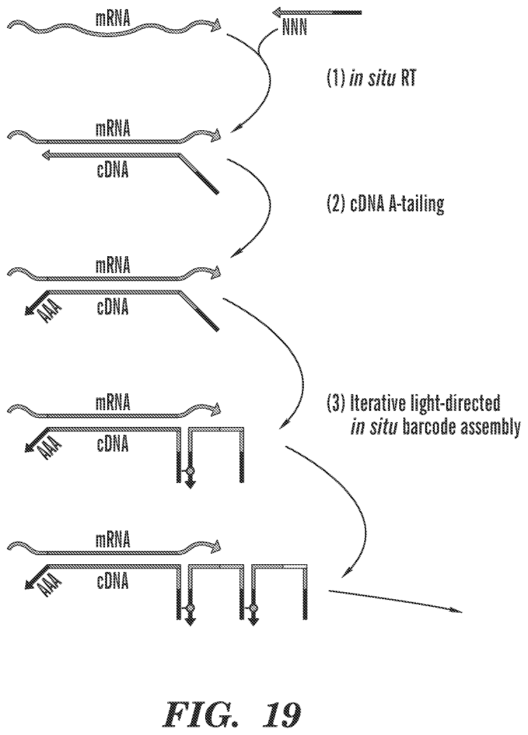

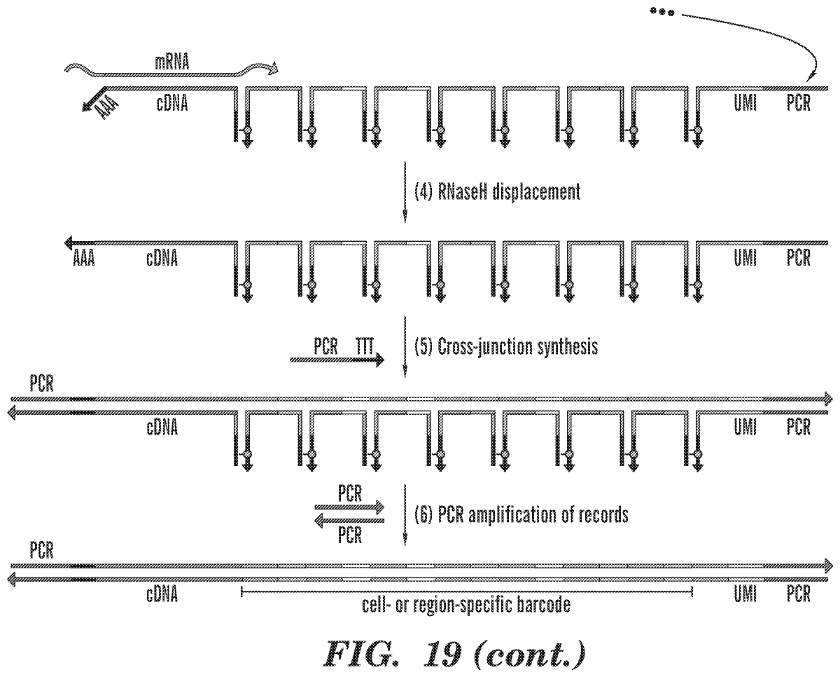

A couple variations of strand diagrams for barcoding of 5′ overhangs of in situ localized nucleic acids are shown in FIG. 18 and FIG. 19.

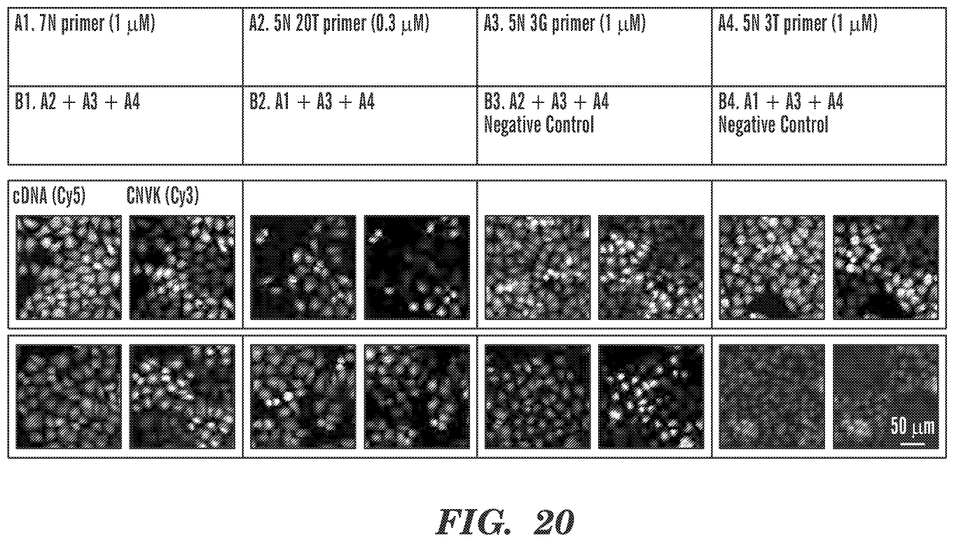

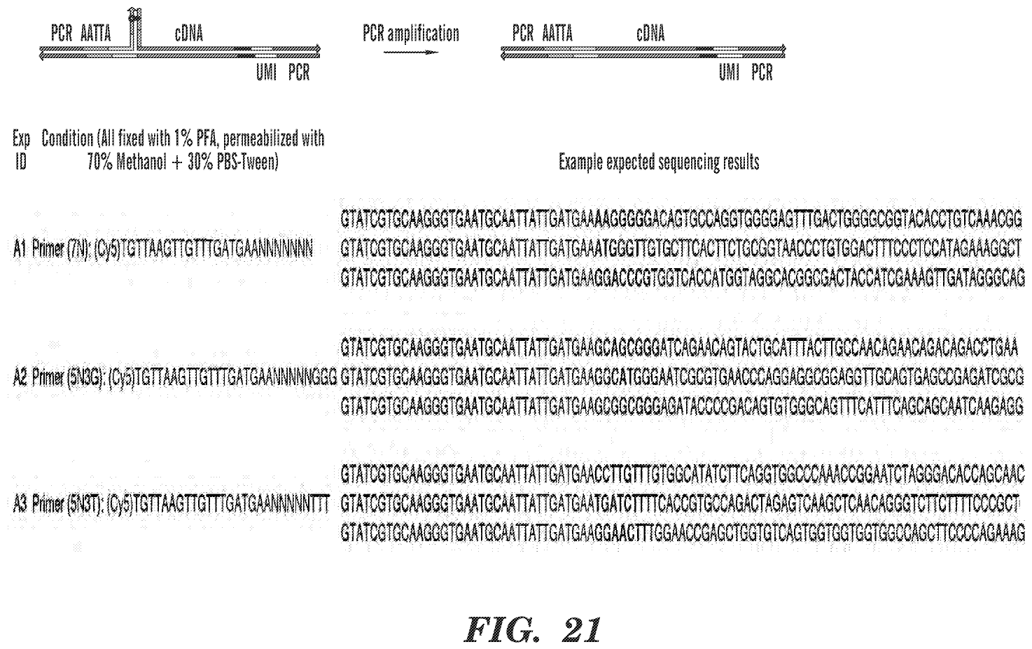

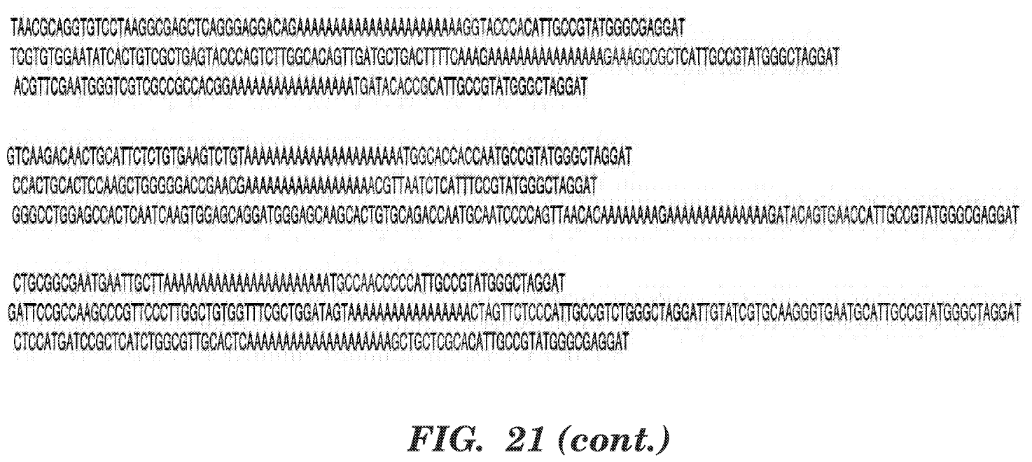

Several different Cy5 labeled primer designs were tested for cDNA library generation (FIG. 20A). HeLa cells were prepared on an Ibidi 8-well chamber and fixed at 1% PFA and permeabilized with 200 ul 70% Methanol and 30% PBS buffer supplemented with 0.1% Tween-20. An identical reverse transcription (RT) protocol was performed on all wells except for the negative controls. Afterwards the primers were imaged in the Cy5 channel to assay their localization (FIG. 20B). Certain primer designs favored the cytoplasmic area indicating that different primers may be accessing and copying different types of RNA species during the RT step. Barcoding of all primer designs was then validated by crosslinking a Cy3 labeled CNVK barcode to the same cells with a checkerboard pattern. Sequencing results for several of these primers are shown in FIG. 21.

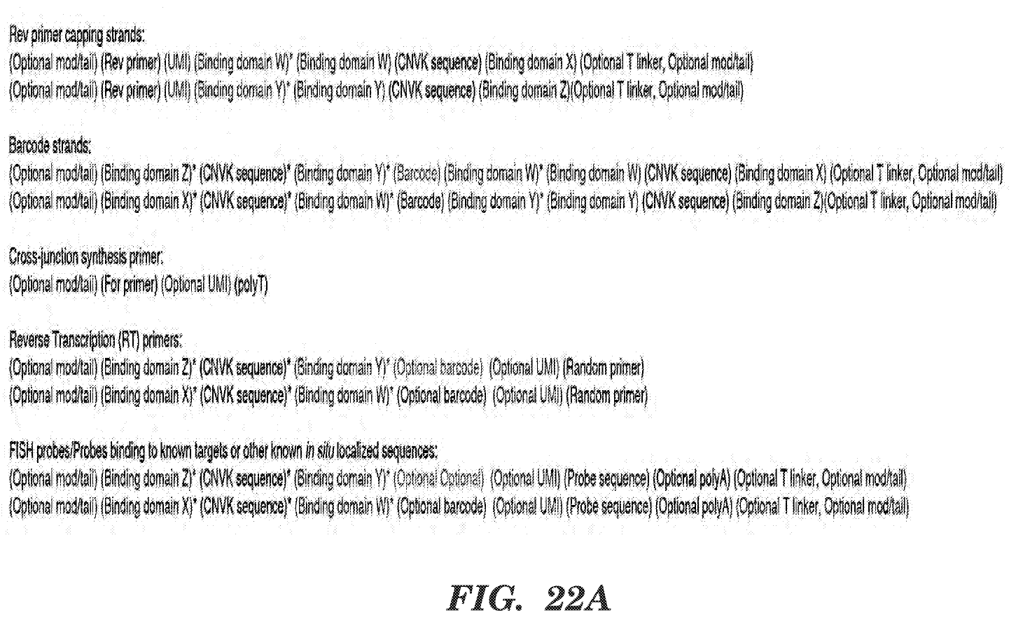

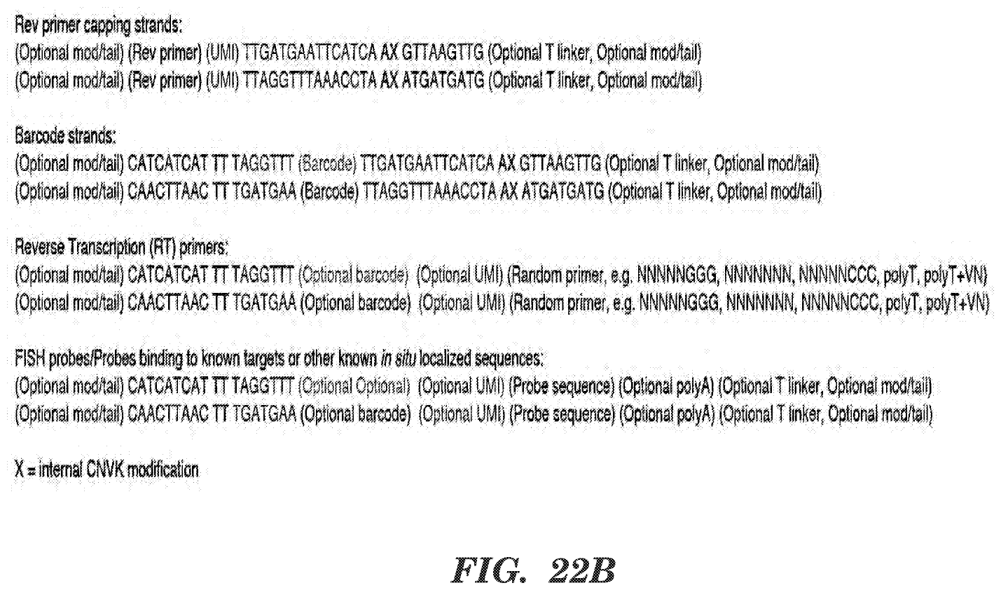

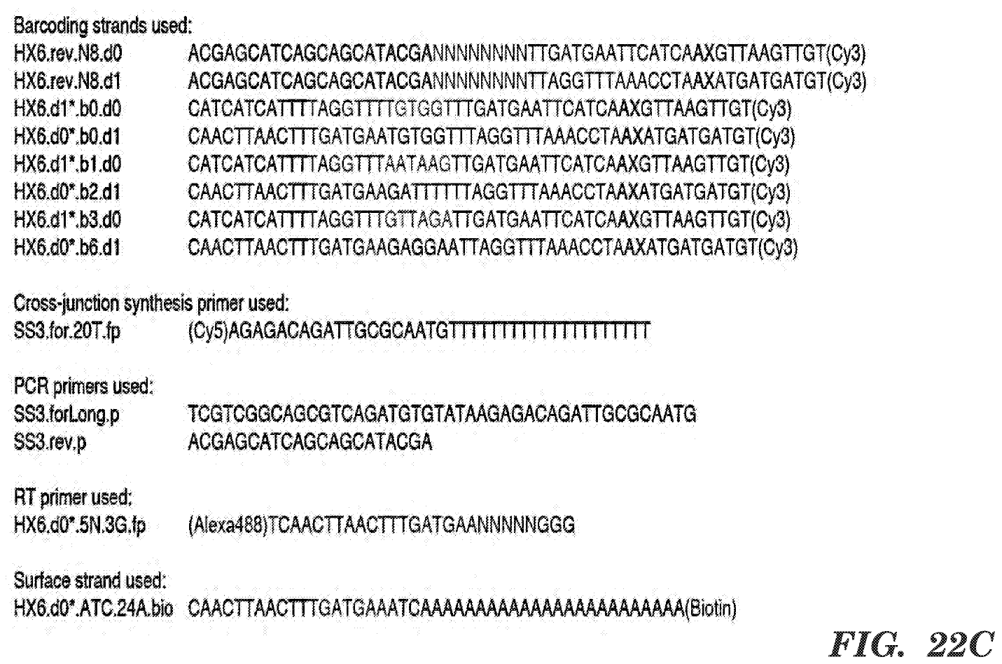

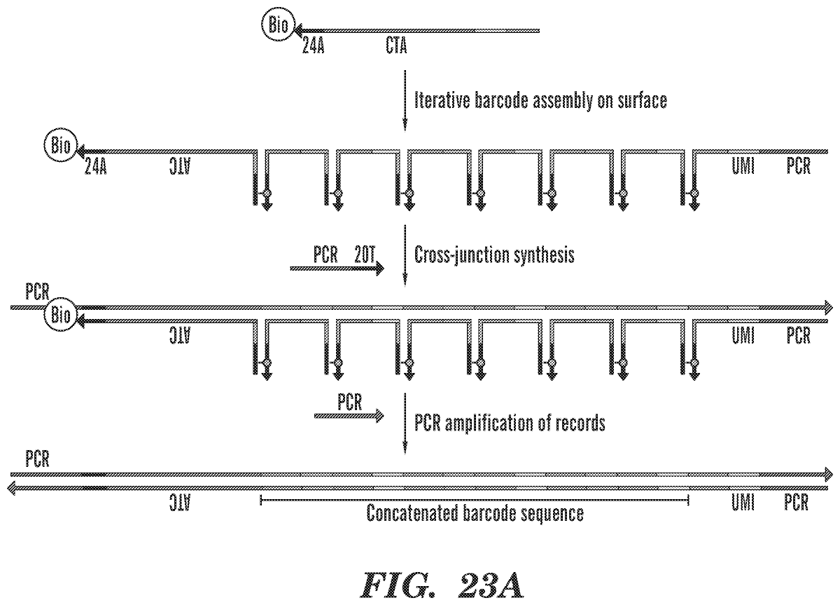

The general sequence design strategy for barcoding of 5′ overhangs of in situ localized nucleic acids is depicted in FIG. 22A and Table 1 below.

TABLE 1
General structures of barcoding strands for two-orientation setup (refer also to FIG. 22A.
Barcoding is done by constructing a concatemer comprising a rev capping barcode strand,
zero or more barcode strands, and a dock strand (e.g.. a RT primer that has been extended
to create a cDNA sequence on RNA, a FISH or other targeted probe, or a strand that has
otherwise been localized in situ to biomolecules via some affinity relationship). In this case,
there are two orientations of sequences, with the orientations alternating every other round
of barcoding. More orientations may also be utilized. Asterisk indicates complementary or
largely complementary domain e.g.. (Binding domain X) hybridizes to (Binding domain X)*.

| Purpose | Sequence Structure |
|---|---|
| Rev capping | (Optional mod/tail) (Rev primer) (UMI) (Binding domain W)* (Binding |
| barcode strand | domain W) (CNVK sequence) (Binding domain X) (Optional T linker, |
| (orientation 1) | Optional mod/tail) |
| Rev capping | (Optional mod/tail) (Rev primer) (UMI) (Binding domain Y)* (Binding |
| barcode strand | domain Y) (CNVK sequence) (Binding domain Z)(Optional T linker, |
| (orientation 2) | Optional mod/tail) |
| Barcode strand | (Optional mod/tail) (Binding domain Z)* (CNVK sequence)* (Binding |
| (orientation 1) | domain Y)* (Barcode) (Binding domain W)* (Binding domain W) (CNVK |
| | sequence) (Binding domain X) (Optional T linker, Optional mod/tail) |
| Barcode strand | (Optional mod/tail) (Binding domain X)* (CNVK sequence)* (Binding |
| (orientation 2) | domain W)* (Barcode) (Binding domain Y)* (Binding domain Y) (CNVK |
| | sequence) (Binding domain Z)(Optional T linker, Optional mod/tail) |
| Cross-junction | (Optional mod/tail) (For primer) (Optional UMI) (polyT) |
| synthesis primer | |
| Reverse | (Optional mod/tail) (Binding domain Z)* (CNVK sequence)* (Binding |
| transcription | domain Y)* (Optional barcode) (Optional UMI) (Random primer) |
| (RT) primer | |
| (orientation 1) | |
| Reverse | (Optional mod/tail) (Binding domain X)* (CNVK sequence)* (Binding |
| transcription | domain W)* (Optional barcode) (Optional UMI) (Random primer) |
| (RT) primer | |
| (orientation 2) | |
| FISH or other | (Optional mod/tail) (Binding domain Z)* (CNVK sequence)* (Binding |
| targeted probe | domain Y)* (Optional Optional) (Optional UMI) (Probe sequence) |
| (orientation 1) | (Optional poly A) (Optional T linker, Optional mod/tail) |
| FISH or other | (Optional mod/tail) (Binding domain X)* (CNVK sequence)* (Binding |
| targeted probe | domain W)* (Optional barcode) (Optional UMI) (Probe sequence) |
| (orientation 2) | (Optional poly A) (Optional T linker, Optional mod/tail) |

The specific binding domain sequences used in subsequent figures are depicted in FIG. 22B and Table 2. In this example, an A base followed by the CNVK modification is paired across from two T nucleotides. It was found that after crosslinking, the CNVK is highly effective at acting as a blocker for polymerase extension, so that it could be used directly as the blocking domain during cross junction synthesis even when all four nucleotides were present (dATP, dTTP, dCTP, dGTP).

TABLE 2
Specific structure of sequences with the d0 and d1 binding domains (refer
also to FIG. 22B). A specific set of barcoding binding domains that have been
experimentally validated are described (d0 = (Binding domain W) described in
Table 1 and d1 = (Binding domain Y) from Table 1). The binding domains must be
designed to be short enough so that non-crosslinked barcode strands may be washed
away without disrupting the underlying affinity or binding of the docking sequence(s)
(e.g.. a cDNA sequence or localized FISH or targeted probe).

| Purpose | Sequence Structure (X = CNVK internal modification) |
|---|---|
| Rev capping barcode | (Optional mod/tail) (Rev primer) (UMI) TTGATGAATTCATCA AX |
| strand (d0 | GTTAAGTTG (Optional T linker, Optional mod/tail) |
| orientation) | |
| Rev capping barcode | (Optional mod/tail) (Rev primer) (UMI) TTAGGTTTAAACCTA AX |
| strand (d1 | ATGATGATG (Optional T linker, Optional mod/tail) |
| orientation) | |
| Barcode strand | (Optional mod/tail) CATCATCAT TT TAGGTTT (Barcode) |
| (d1*-> d0 orientation) | TTGATGAATTCATCA AX GTTAAGTTG (Optional T linker, |
| | Optional mod/tail) |
| Barcode strand | (Optional mod/tail) CAACTTAAC TT TGATGAA (Barcode) |
| (d0*->d1 orientation) | TTAGGTTTAAACCTA AX ATGATGATG (Optional T linker, |
| | Optional mod/tail) |
| Reverse | (Optional mod/tail) CATCATCAT TT TAGGTTT (Optional barcode) |
| transcription (RT) | (Optional UMI) (Random primer, e.g.. NNNNNGGG, NNNNNNN, |
| primer (d1* | NNNNNCCC, polyT, polyT + VN) |
| orientation) | |
| Reverse | (Optional mod/tail) CAACTTAAC TT TGATGAA (Optional barcode) |
| transcription (RT) | (Optional UMI) (Random primer, e.g.. NNNNNGGG, NNNNNNN, |
| primer (d0* | NNNNNCCC, polyT, polyT + VN) |
| orientation) | |
| FISH or other | (Optional mod/tail) CATCATCAT TT TAGGTTT (Optional Optional) |
| targeted probe (d1* | (Optional UMI) (Probe sequence) (Optional poly A) (Optional T linker, |
| orientation) | Optional mod/tail) |
| FISH or other | (Optional mod/tail) CAACTTAAC TT TGATGAA (Optional barcode) |
| targeted probe (d0* | (Optional UMI) (Probe sequence) (Optional poly A) (Optional T linker, |
| orientation) | Optional mod/tail) |

FIG. 22C and Table 3 show the exact barcoding and primer sequences used in subsequent figures.

TABLE 3
Experimentally validated sequences (refer also to FIG. 22C and data figures).
Barcode sequences validated with full barcoding workflow, including sequencing.

| | Sequence used (X = CNVK | | |
|---|---|---|---|
| | internal modification, Bolded | Name of | |
| Purpose | underline = barcode domain) | sequence | SEQ ID NO: |
| Rev capping barcode | ACGAGCATCAGCAGCATA | rev.N8.d0 | SEQ ID NO: 1 |
| strand (d0 | CGANNNNNNNNTTGATGA | | |
| orientation) | ATTCATCAAXGTTAAGTT | | |
| | GT(Cy3) | | |
| Rev capping barcode | ACGAGCATCAGCAGCATA | rev.N8.d1 | SEQ ID NO: 2 |
| strand (d1 | CGANNNNNNNNTTAGGTT | | |
| orientation) | TAAACCTAAXATGATGAT | | |
| | GT(Cy3) | | |
| Barcode strand | CATCATCATTTTAGGTTTT | d1*.bO.d0 | SEQ ID NO: 3 |
| (d1*->d0 orientation, | GTGGTTTGATGAATTCAT | | |
| barcode 0 = | CAAXGTTAAGTTGT(Cy3) | | |
| TGTGGT) | | | |
| Barcode strand | CAACTTAACTTTGATGAA | d0*.bO.d1 | SEQ ID NO: 4 |
| (d0*->d1 orientation, | TGTGGTTTAGGTTTAAAC | | |
| barcode 0 = | CTAAXATGATGATGT(Cy3) | | |
| TGTGGT) | | | |
| Barcode strand | CATCATCATTTTAGGTTTA | d1*.b1.d0 | SEQ ID NO: 5 |
| (d1*->d0 orientation, | ATAAGTTGATGAATTCAT | | |
| barcode 1 = | CAAXGTTAAGTTGT(Cy3) | | |
| AATAAG) | | | |
| Barcode strand | CAACTTAACTTTGATGAA | d0*.b2.d1 | SEQ ID NO: 6 |
| (d0*->d1 orientation, | GATTTTTTAGGTTTAAAC | | |
| barcode 2 = | CTAAXATGATGATGT(Cy3) | | |
| GATTTT) | | | |
| Barcode strand | CATCATCATTTTAGGTTTG | d1*.b3.d0 | SEQ ID NO: 7 |
| (d1*-> d0 orientation, | TTAGATTGATGAATTCAT | | |
| barcode 3 = | CAAXGTTAAGTTGT(Cy3) | | |
| GTTAGA) | | | |
| Barcode strand | CAACTTAACTTTGATGAA | d0*.b6.d1 | SEQ ID NO: 8 |
| (d0*->d1 orientation, | GAGGAATTAGGTTTAAAC | | |
| barcode 6 = | CTAAXATGATGATGT(Cy3) | | |
| GAGGAA) | | | |
| Cross-junction | (Cy5)AGAGACAGATTGCG | for.20T.fp | SEQ ID NO: 9 |
| synthesis primer | CAATGTTTTTTTTTTTTTTT | | |
| | TTTTT | | |
| Surface strand (d0* | CAACTTAACTTTGATGAA | HX6.d0*.ATC. | SEQ ID NO: 10 |
| orientation, ATC | ATCAAAAAAAAAAAAAA | 24A.bio | |
| internal, polyA tail) | AAAAAAAAAA(Biotin) | | |

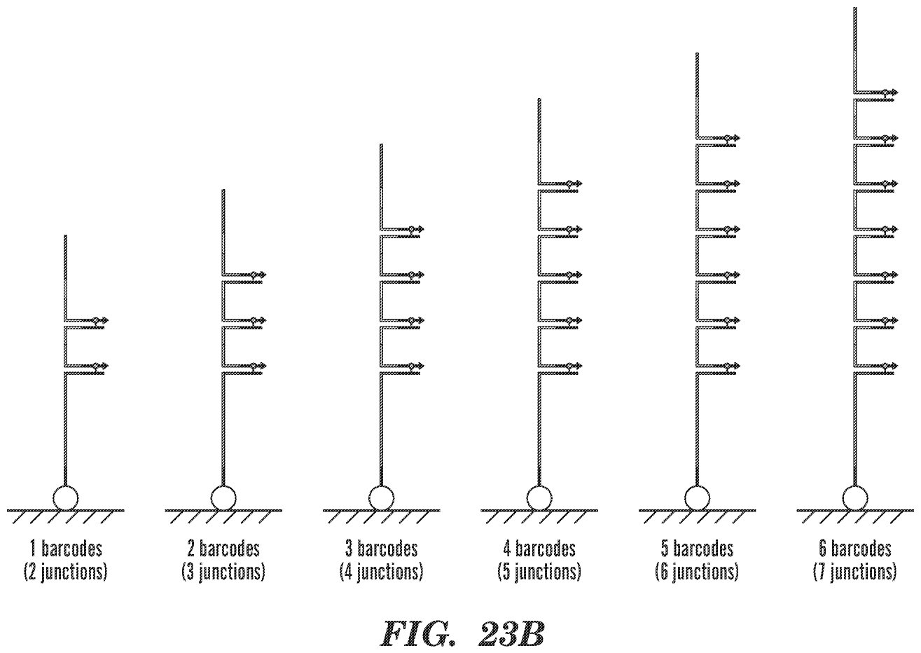

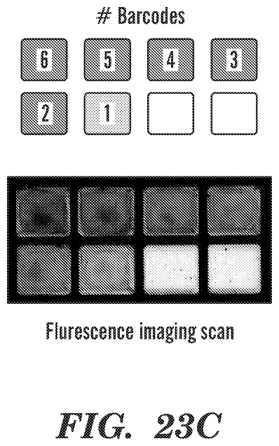

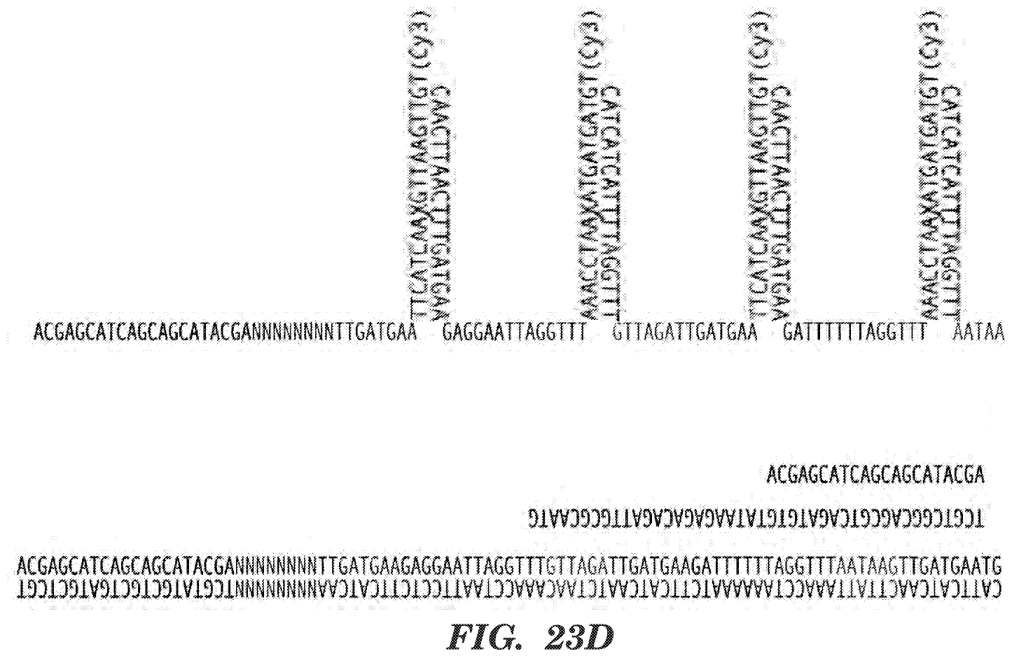

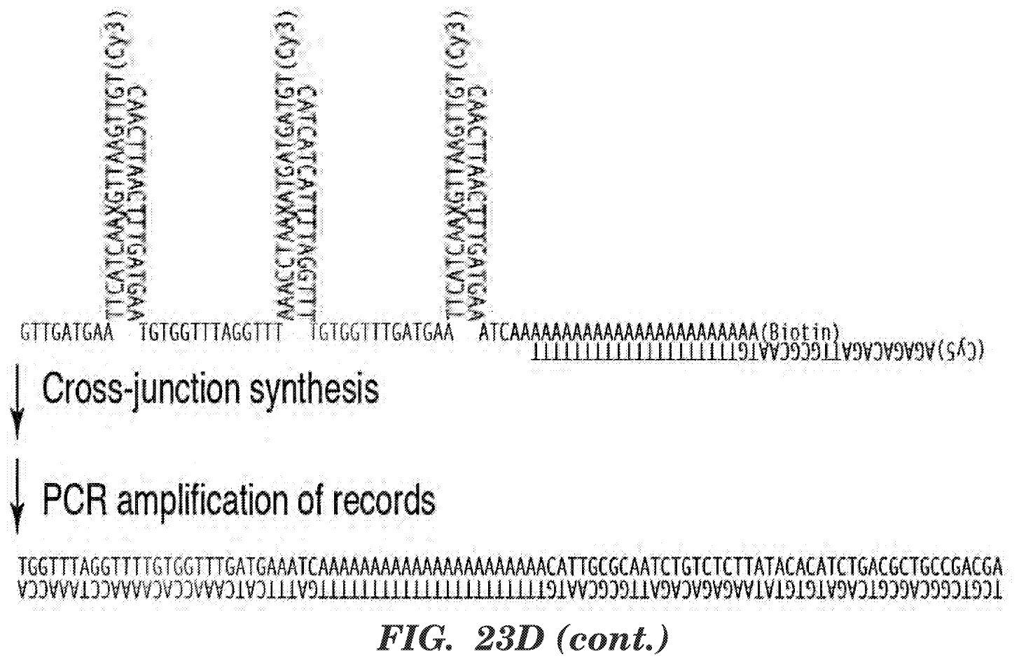

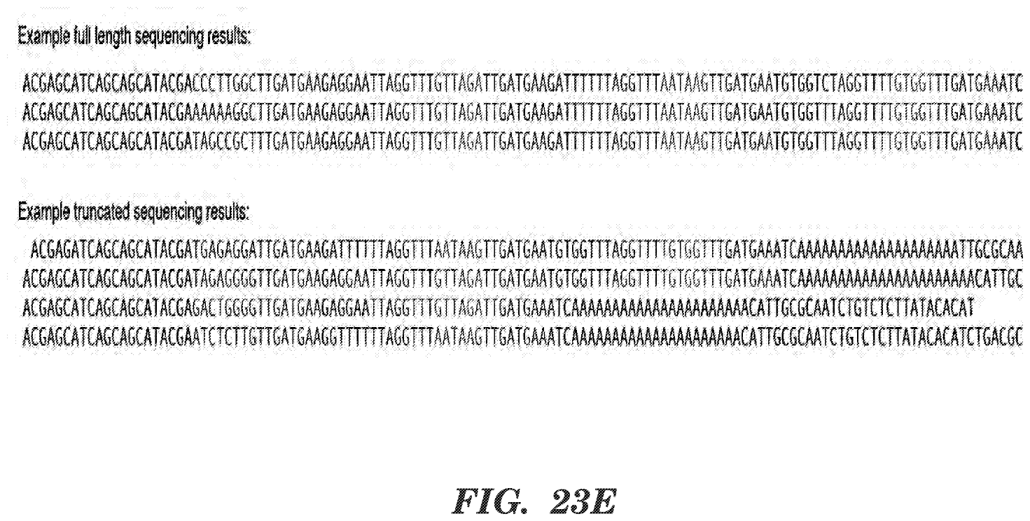

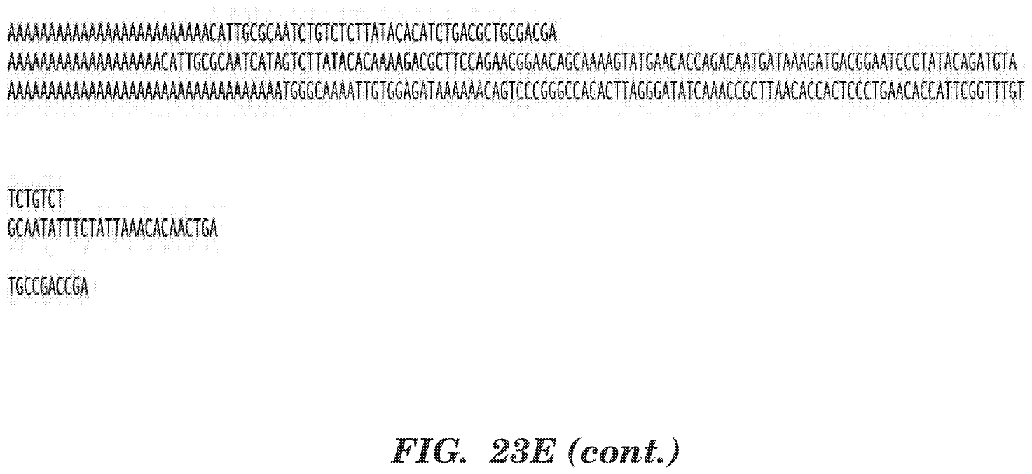

These sequences were tested through the concatenation of up to 8 strands together (to form 7 junctions) via iterative barcoding of a biotinylated strand bound to a streptavidin coated glass slide (FIGS. 23A-23B). In each of 6 wells, a different number of barcodes was introduced to create between 2 and 7 junctions for cross junction synthesis (FIG. 23C). The complete expected junction and amplicon sequence design is depicted in FIG. 23D, with the expected crosslinking sites indicated from the CNVK (“X”) modification. Some full sequences with the expected six-barcodes were identified after sequencing, as well as a majority of truncated four-, two-, and zero-barcode sequences indicating imperfect assembly efficiency. The vast majority of reads showed barcodes in the correct order of introduction, indicating that the barcode records do indeed reflect the temporal introduction of specific barcode sequences.

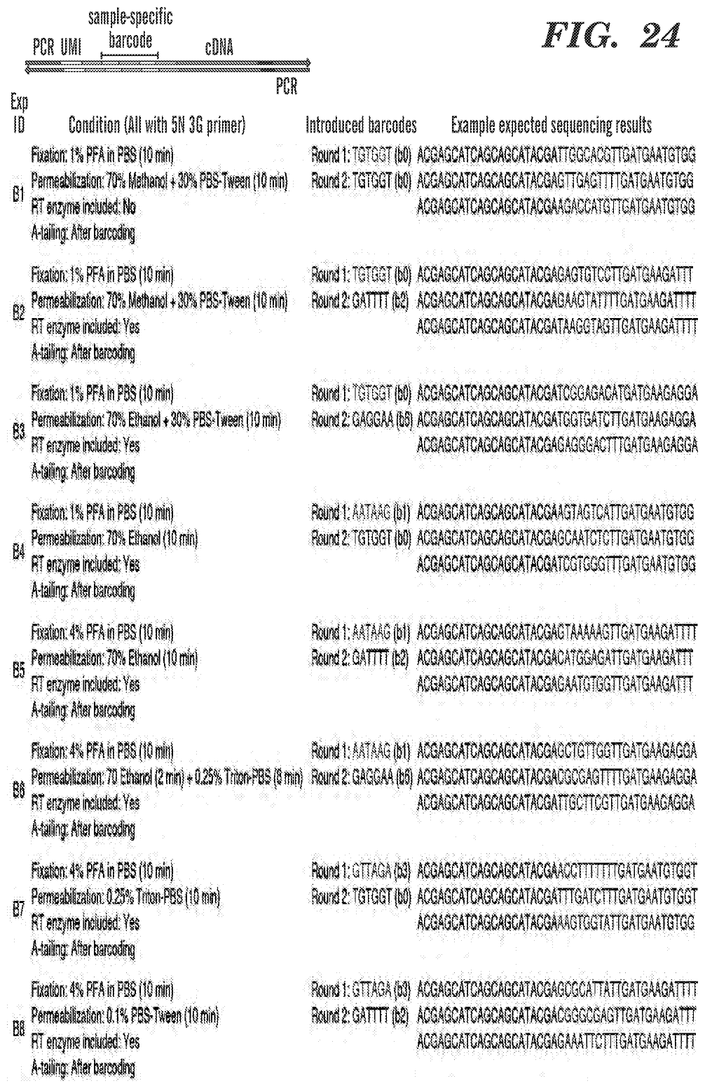

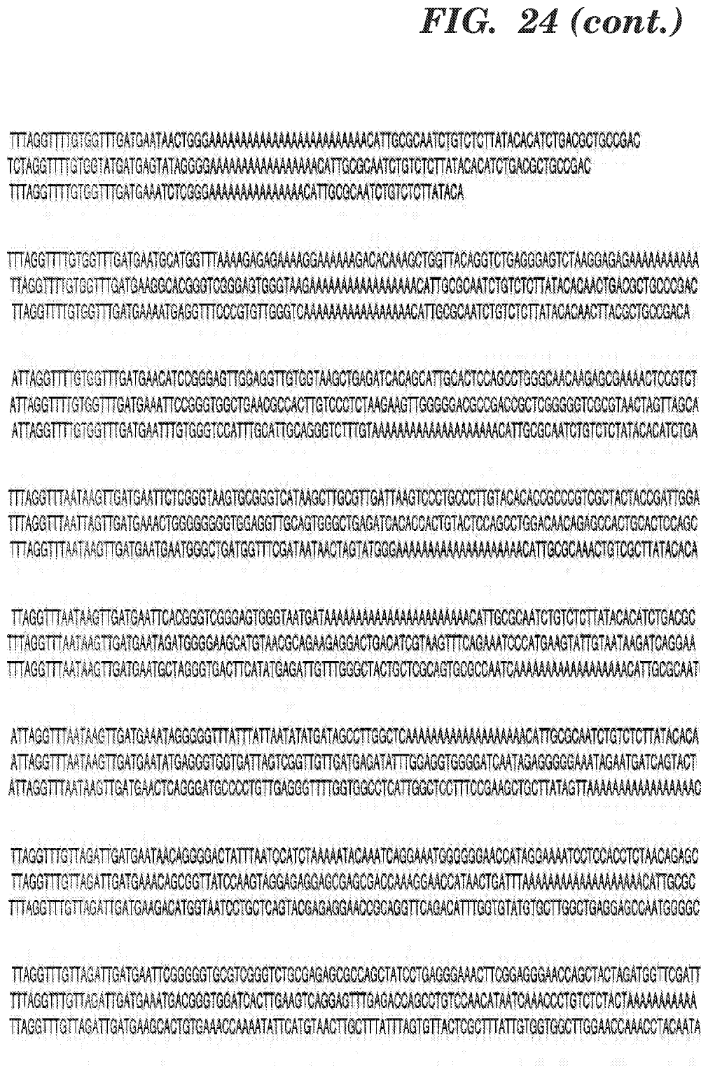

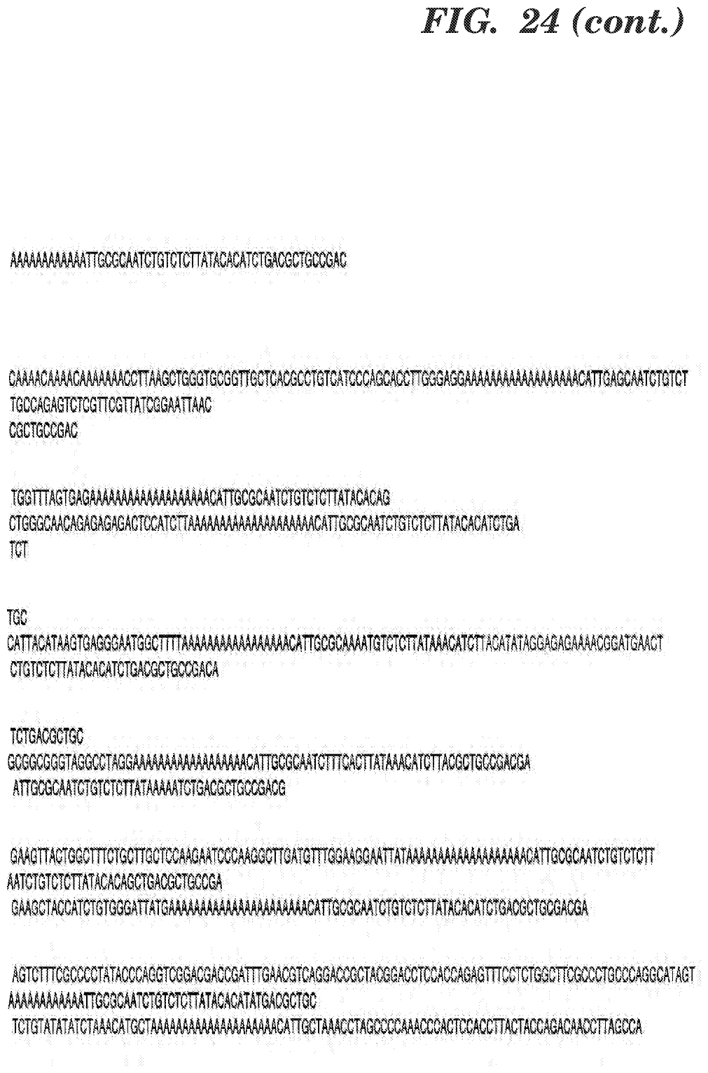

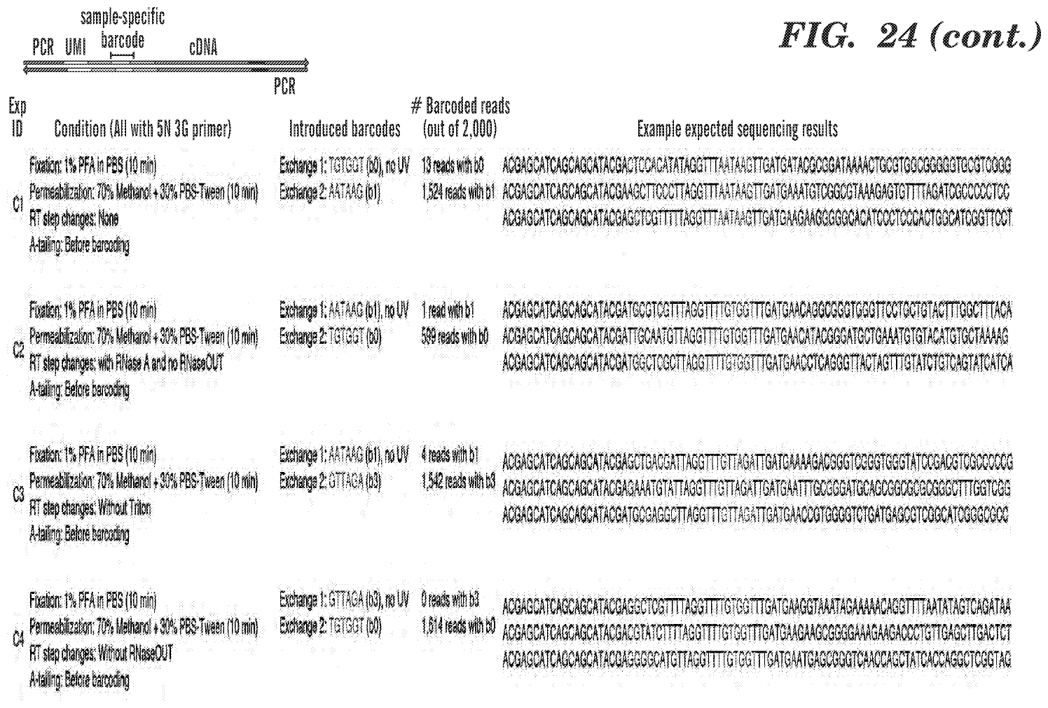

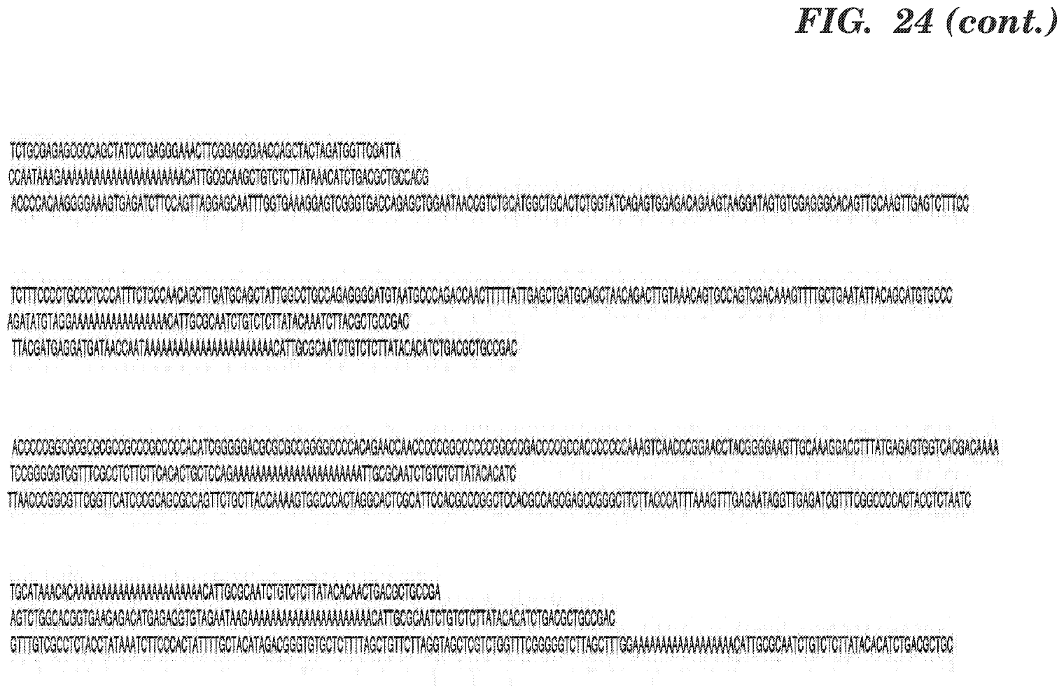

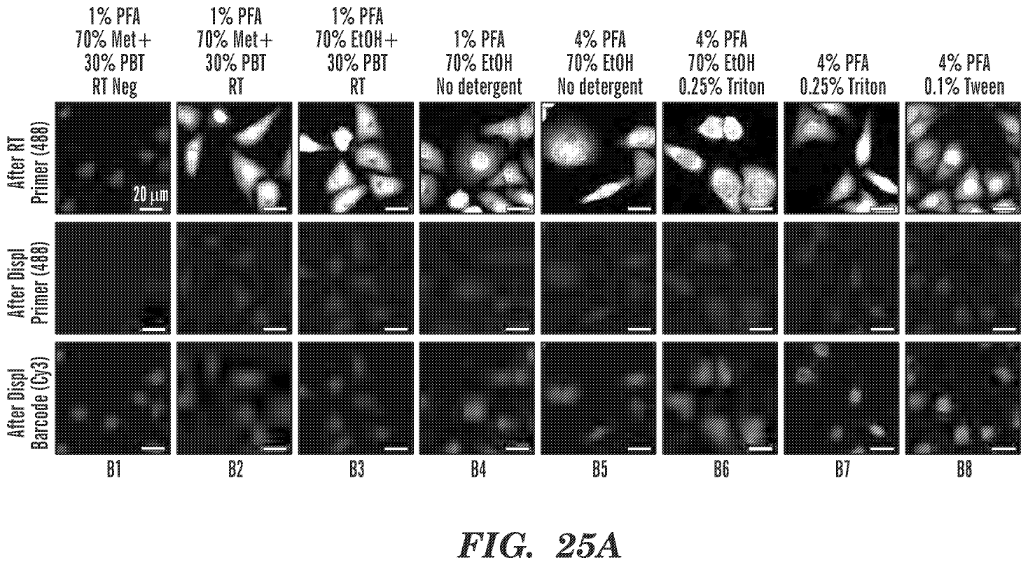

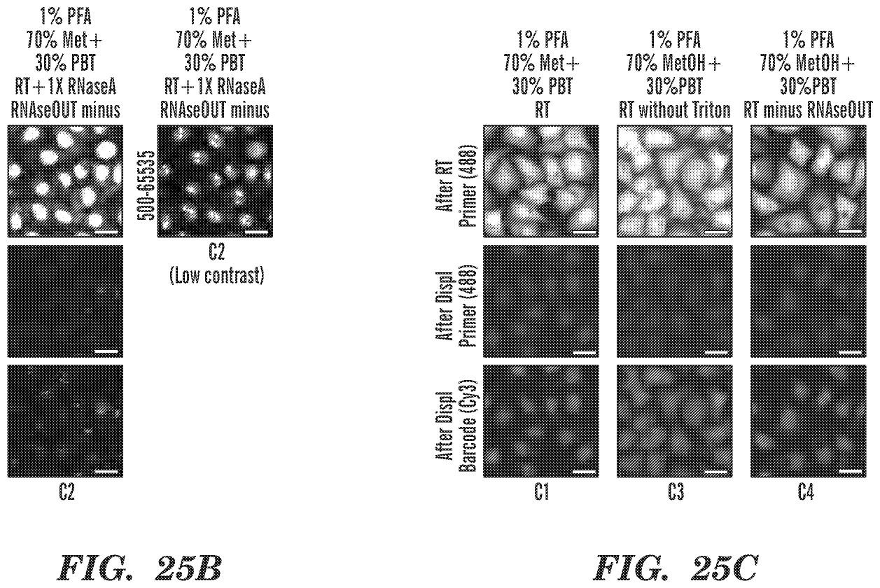

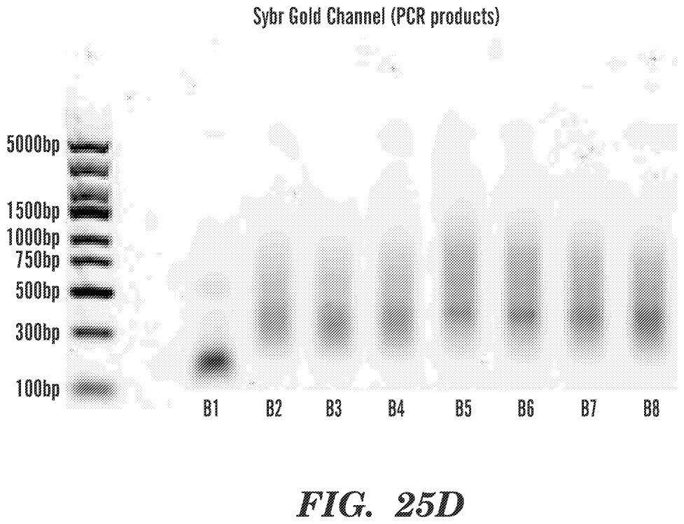

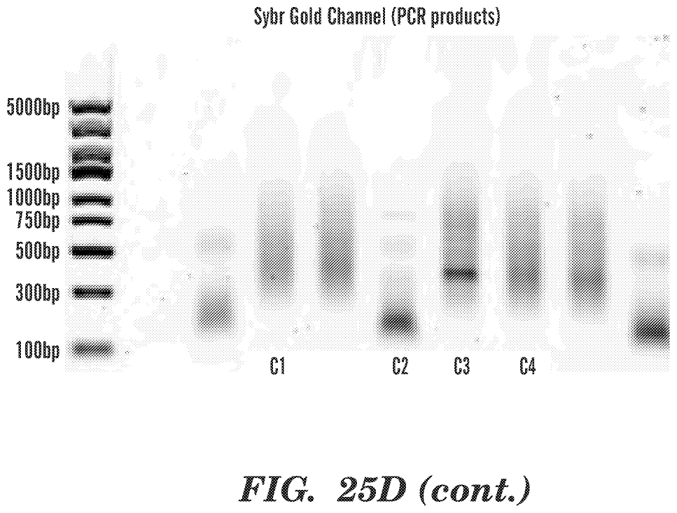

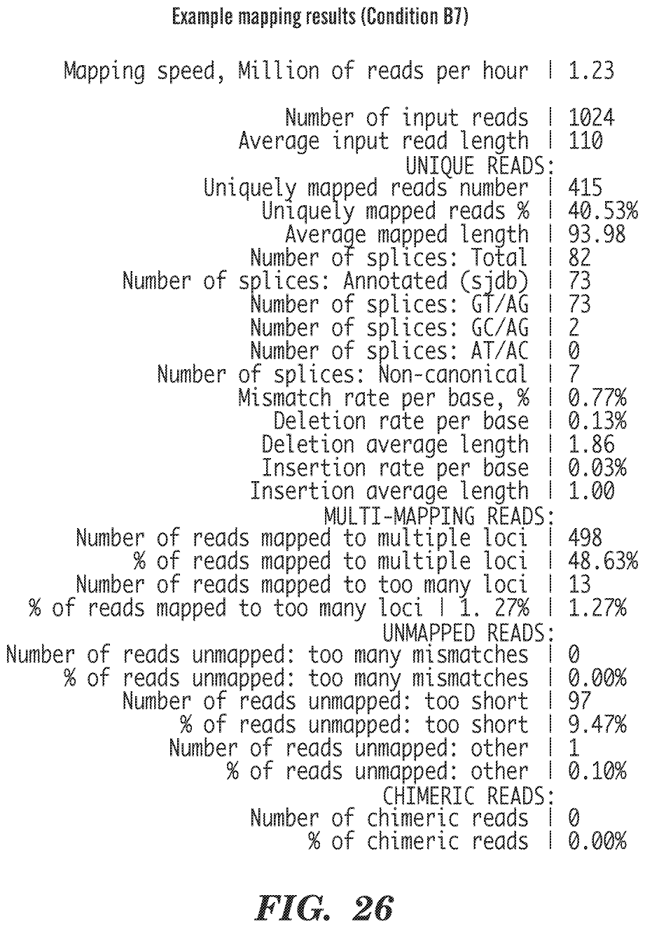

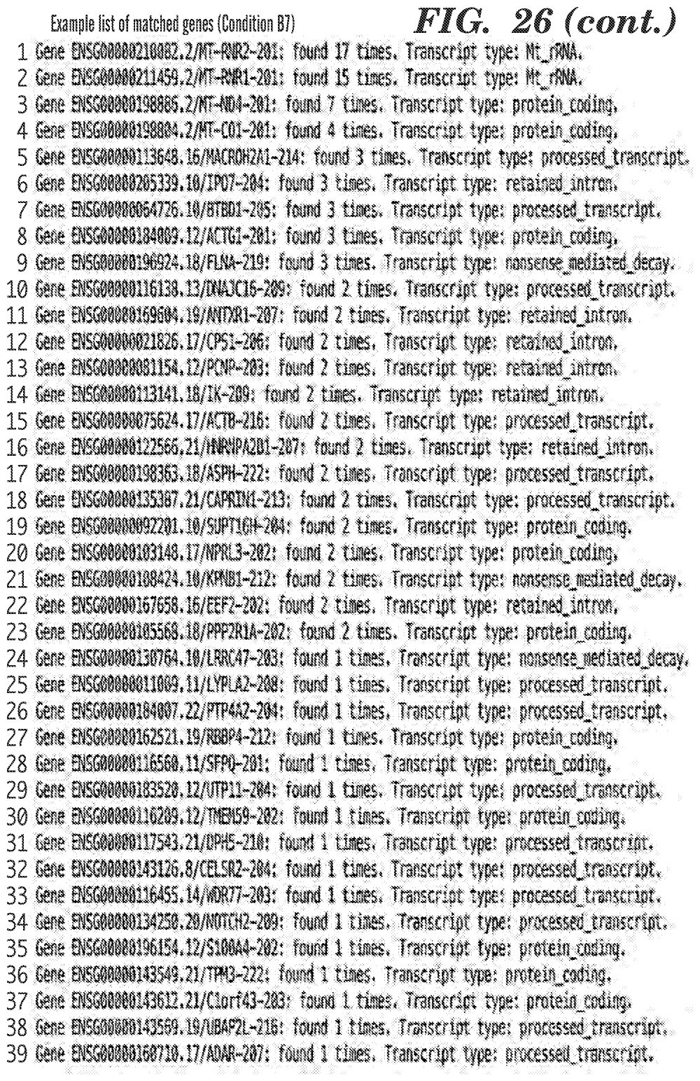

These sequences were then applied for barcoding cDNA sequences in fixed HeLa cells following the workflow described in FIG. 19. A variety of fixation, permeabilization, RT, A-tailing, and barcoding conditions were tested together and all conditions shown produced some expected sequencing results (FIG. 24) In the sequencing results shown, except for the control missing the RT enzyme, the highlighted cDNA reads (blue) mapped to known Homo sapiens sequences. Together, these data indicate the robustness of the technique under a wide range of RT, permeabilization, and fixation conditions. The results from experiments C1 through C4 further show that background from barcodes introduced in previous rounds but not crosslinked is very little, as very few reads are seen for those earlier barcodes. This indicates that the chosen stringent wash condition (40% formamide in either 1×PBS or 1×PBS-Tween) is sufficient to wash away bound, but non-crosslinked, barcode strands. Imaging and gel results for these same experiments (B1 through B8 and C1 through C4) are shown in FIG. 25A-25D. An example of successful gene sequence mapping results for 1,024 parsed reads of the correct sequence format from one of these experiments (B7) is shown in FIG. 26.

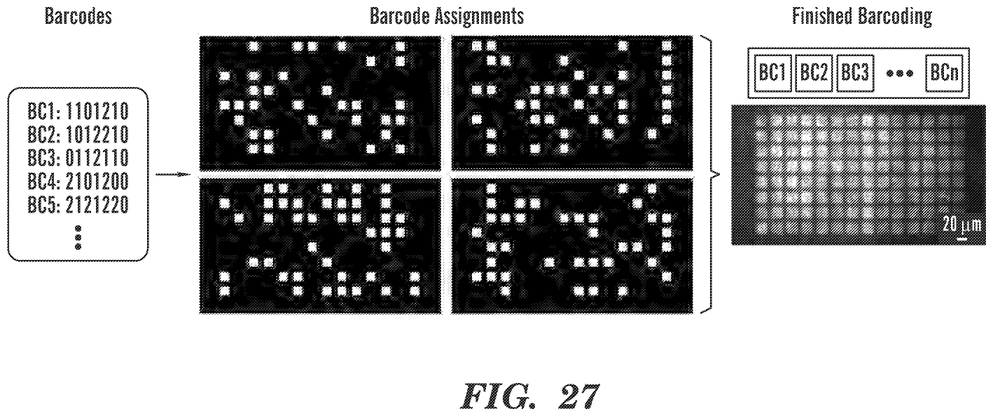

An experimental test of the combinatorial barcoding strategy was performed using a set of six DNA barcodes and integrated with an automated fluidic exchange unit as well as a control macro to adjust photomasks per barcoding round (FIG. 27). A total of 112 square sized regions of interest were assigned a unique DNA barcode sequence. Our encoding strategy leverages a trinary encoding scheme (0, 1, 2). A total of six barcoding rounds were performed followed by a final capping round adding a rev primer strand. Each barcode round is assigned a unique photomask to parallelize barcode assignments within the field of view. Following the final capping strand addition, an image was taken in the Cy3 channel to visualize successful barcode incorporation.

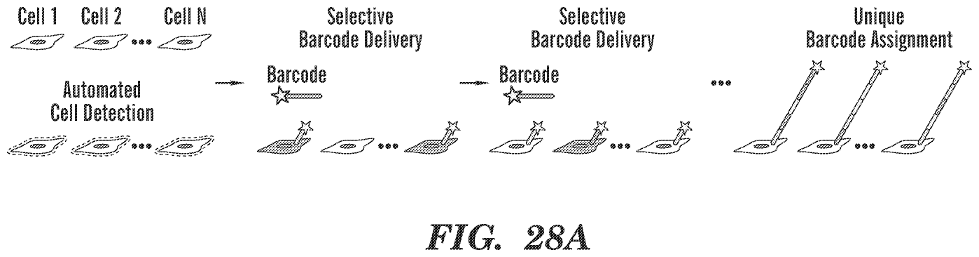

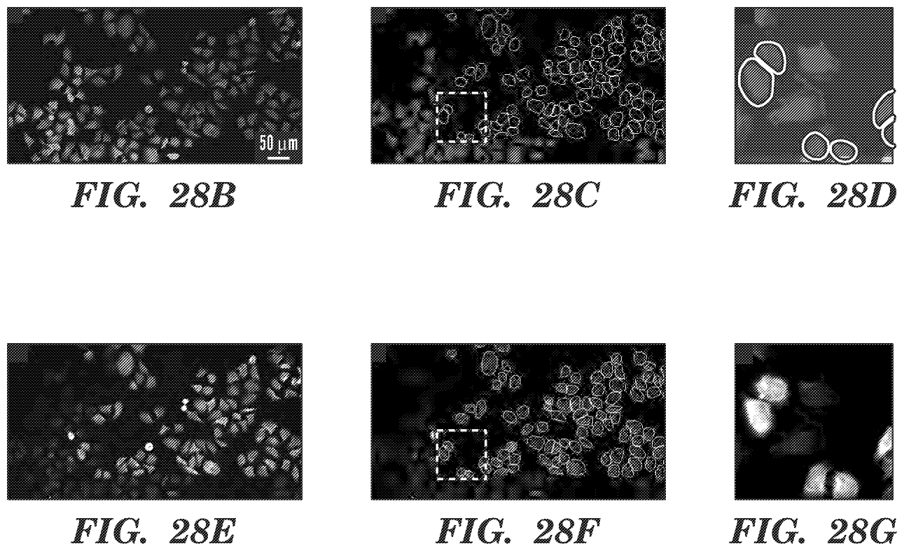

An experimental test of an integrated automated cell detection, photomasking and barcoding workflow (FIG. 28A). HeLa cells were seeded in an Ibidi flow chamber and fixed with 4% PFA and permeabilized in 1×PBS supplemented with 0.25% Triton-X. A 5N.3G (see FIG. 22A-22C) primer was used for reverse transcription and imaged in the Cy5 channel (FIG. 28B). An algorithm was used to identify cells based on the Cy5 signal and the outlines of the detected cells are overlaid on the Cy5 signal (FIG. 28C). Each cell outline functions as a region of interest, and is assigned its own unique barcode sequence. Automated barcoding and DNA barcode exchange was performed on the selected cells using a Cy3 labeled CNVK DNA barcode. Afterwards cells were imaged in the Cy3 channel (FIG. 28E) to confirm successful barcode delivery.

The workflow provided in FIG. 16 can be used to barcode biomolecules in pre-defined regions of interest (such as whole tissues, tissue regions, collection of cells, single cells, subcellular regions, microbes, surfaces) in order to tag them for multimodal integrated analysis by both imaging based methods and by sequencing-based methods. In this case, the methods provided herein are used to create spatial tags that relate sequencing reads back to spatial positions that they originate from. Hence the barcoding methods allow screening libraries of candidate treatments (small molecule drugs, therapeutic nucleic acids, gene or cell therapies, peptides, proteins, antibodies, metabolites, hormones, DNA encoded libraries) where phenotypic outcomes are identified by imaging and selected regions are barcoded by light exposure for sequencing based analysis using the methods provided herein. The applications of the method include and are not limited to screening of therapies (small molecule drugs, biologics, therapeutic nucleic acids, gene or cell therapies, siRNAs, gRNAs, peptides, proteins, antibodies, metabolites, hormones, DNA encoded libraries), identification of drug targets, identification of biomarkers, profiling, characterization of phenotypic to genotypic cell state, generation of new disease models, characterization of cells and disease models, characterization of differentiation status and cell state, tissue mapping, multi-dimensional analysis, high content screening, machine-learning based clustering or classification, cell therapy development, CAR-T therapy development, antibody screening, personalized medicine, cell enrichment.

The method can be applied to any pre-existing target nucleic acid and other biomolecules that are either directly conjugated to a nucleic acid or indirectly bound to a nucleic acid via adaptors such as affinity binders, antibodies, nanobodies aptamers, affibodies, tags, fusion proteins, linkers. In this case potential target molecules includes and are not limited to DNA encoded libraries of small molecules, peptides, proteins, antibodies, ligands, plasmids, siRNAs, guide (gRNAs), plasmids, phages, viruses, metabolites, hormones, and DNA-barcoded surfaces, subcellular structures or whole cells or microorganisms.

The method provided herein can be used to linearly or combinatorially barcode biomolecules with crosslinked DNA strands by using any of the compositions provided herein and exposing the molecules in pre-defined regions of interest to light.

For example, the method can be used to barcode biomolecules in pre-defined regions of interest (whole tissues, tissue regions, collection of cells, single cells, subcellular regions, microbes, surfaces) in order to tag them for multimodal integrated analysis by both imaging based methods and by sequencing-based methods.

Furthermore, barcoding biomolecules to create spatial tags that relate sequencing reads back to spatial positions can be achieved for multimodal integrated analysis of selected regions of interest both imaging based methods and by sequencing-based methods

The workflow in FIG. 16 can also be used for screening libraries of candidate treatments for various diseases. For example, screening of small molecule drugs, biologics, therapeutic nucleic acids, gene or cell therapies, siRNAs, gRNAs, plasmids, phages, viruses, peptides, proteins, antibodies, metabolites, hormones, and DNA encoded libraries. The phenotypic outcomes are identified by imaging and selected regions that are barcoded by light exposure for sequencing based analysis using the methods provided herein.

The methods provided herein can be advantageous for the various applications including but not limited to the identification of drug targets, identification of biomarkers, profiling, characterization of phenotypic to genotypic cell state, generation of new disease models, characterization of cells and disease models, characterization of differentiation status and cell state, tissue mapping, multi-dimensional analysis, high content screening, machine-learning based clustering or classification, cell therapy development, CAR-T therapy development, antibody screening, personalized medicine, and cell enrichment.

### REFERENCES

1) S. Picelli et al, Nat. Methods 10, 1096-1098 (2013).
2) T. Hashimshony, F. Wagner, N. Sher, I. Yanai, Cell Reports 2, 666-673 (2012).
3) D. A. Jaitin et al., Science 343, 776-779 (2014).
4) Z. Macosko et al., Cell 161, 1202-1214 (2015).
5) M. Klein et al., Cell 161, 1187-1201 (2015).
6) G. X. Y. Zheng et al, Nat. Commun. 8, 14049 (2017).
7) P. L. Stahl et al., Science 353, 78-82 (2016).
8) Rodrigues, S. G., Stickels, R. R., Goeva, A., Martin, C. A., Murray, E., Vanderburg, C. R., . . . & Macosko, E. Z. (2019). Slide-seq: A scalable technology for measuring genome-wide expression at high spatial resolution. Science, 363(6434), 1463-1467.
9) Rosenberg, A. B., Roco, C. M., Muscat, R. A., Kuchina, A., Sample, P., Yao, Z., . . . & Pun, S. H. (2018). Single-cell profiling of the developing mouse brain and spinal cord with split-pool barcoding. Science, 360(6385), 176-182.
10) Vickovic, S., Eraslan, G., Klughammer, J., Stenbeck, L., Salmen, F., Aijo, T., . . . & Frisen, J. (2019). High-density spatial transcriptomics arrays for in situ tissue profiling. bioRxiv, 563338.
11) Fazal, Furqan M., Shuo Han, Kevin R. Parker, Pornchai Kaewsapsak, Jin Xu, Alistair N. Boettiger, Howard Y. Chang, and Alice Y. Ting. “Atlas of subcellular RNA localization revealed by APEX-seq.” Cell (2019).
12) Sundah, Noah R., Nicholas R Y Ho, Geok Soon Lim, Auginia Natalia, Xianguang Ding, Yu Liu, Ju, Ee Seet, Ching Wan Chan, Tze Ping Loh, and Huilin Shao. “Barcoded DNA nanostructures for the multiplexed profiling of subcellular protein distribution.” Nature biomedical engineering (2019): 1-11.
13) Femino, A. M., Fay, F. S., Fogarty, K. & Singer, R. H. Visualization of single RNA transcripts in situ. Science 280, 585-590 (1998).
14) Raj, A., van den Bogaard, P., Rifkin, S. A., van Oudenaarden, A. & Tyagi, S. Imaging individual mRNA molecules using multiple singly labeled probes. Nat. Methods 5, 877-879 (2008).
15) Kishi, J. Y., Lapan, S. W., Beliveau, B. J., West, E. R., Zhu, A., Sasaki, H. M., Saka, S. K., Wang, Y., Cepko, C. L. and Yin, P., 2019. SABER amplifies FISH: enhanced multiplexed imaging of RNA and DNA in cells and tissues. Nature methods, 16(6), p. 533 (2019)
16) Lubeck, E. & Cai, L. Single-cell systems biology by super-resolution imaging and combinatorial labeling. Nat. Methods 9, 743-748 (2012).
17) Lubeck, E., Coskun, A. F., Zhiyentayev, T., Ahmad, M. & Cai, L. Single-cell in situ Profiling the transcriptome with RNA SPOTs. Nat. Methods 14, 1153-1155 (2017).
18) Eng, C. L., Shah, S., Thomassie, J. & Cai, L. Shah, S. et al. Dynamics and spatial genomics of the nascent transcriptome by intron seqFISH. Cell 174, 363-376.e16 (2018).
19) Eng, C. H. L., Lawson, M., Zhu, Q., Dries, R., Koulena, N., Takei, Y., . . . & Cai, L. (2019). Transcriptome-scale super-resolved imaging in tissues by RNA seqFISH+. Nature, 568(7751), 235.
20) Kerstens, H. M., Poddighe, P. J. & Hanselaar, A. G. A novel in situ hybridization signal amplification method based on the deposition of biotinylated tyramine. J. Histochem. Cytochem. 43, 347-352 (1995).
21) Dirks, R M. & Pierce, N. A. Triggered amplification by hybridization chain reaction. Proc. Natl Acad. Sci. USA 101, 15275-15278 (2004).
22) Choi, H. M. T. et al. Programmable in situ amplification for multiplexed imaging of mRNA expression. Nat. Biotechnol. 28, 1208-1212 (2010).
23) Choi, H. M., Beck, V. A. & Pierce, N. A. Next-generation in situ hybridization chain reaction: higher gain, lower cost, greater durability. ACS Nano 8, 4284-4294 (2014).
24) Rouhanifard, S. H. et al. ClampFISH detects individual nucleic acid molecules using click chemistry-based amplification. Nat. Biotechnol. 37, 84-89 (2018).
25) Nagendran, M., Riordan, D. P., Harbury, P. B. & Desai, T. J. Automated cell-type classification in intact tissues by single-cell molecular profiling. Elife 7 (2018).
26) Player, A. N., Shen, S. P., Kenny, D., Antao, V. P. & Kolberg, J. A. Single-copy gene detection using branched DNA (bDNA) in situ hybridization. J. Histochem. Cytochem. 49, 603-611 (2001).
27) Xia, C., Babcock, H. P., Moffitt, J. R. and Zhuang, X., 2019. Multiplexed detection of RNA using MERFISH and branched DNA amplification. Scientific reports, 9(1), p. 7721.
28) Wang, F. et al. RNAscope: a novel in situ RNA analysis platform for formalin-fixed, paraffin-embedded tissues. J. Mol. Diagn. 14, 22-29 (2012).
29) Shah, S. et al. Single-molecule RNA detection at depth via hybridization chain reaction and tissue hydrogel embedding and clearing. Development 92, 2862-2867 (2016).
30) Chen, F., Tillberg, P. W. & Boyden, E. S. Expansion microscopy. Science 347, 543-548 (2015).
31) Wang . . . Zhuang et al, Scientific Reports (2018)
32) Chen et al., Science (2015)
33) Ke, R. et al. In situ sequencing for RNA analysis in preserved tissue and cells. Nat. Methods 10, 857-860 (2013).
34) Lee, J. H. et al. Highly multiplexed subcellular RNA sequencing in situ. Science (80-.). 343, 1360-1363 (2014).
35) Iyer, Eswar Prasad Ramachandran, et al. “Barcoded oligonucleotides ligated on RNA amplified for multiplex and parallel in-situ analyses.” bioRxiv (2018): 281121.
36) Wang . . . Deisseroth et al., Science (2018)
37) Liu N., Dai M., Saka S. K., Yin P. Super-resolution labelling with Action-PAINT. Nature Chemistry (2019), in press.
38) Kim, S. H., Liu, Y., Hoelzel, C., Zhang, X., & Lee, T. H. (2019). Super-Resolution Optical Lithography with DNA. Nano letters.
39) Lieberman-aiden, E. et al. Comprehensive Mapping of Long-Range Interactions Reveals Folding Principles of the Human Genome. Science (80-.). 326, 289-293 (2009).
40) Schaus, T. E., Woo, S., Xuan, F., Chen, X., & Yin, P. (2017). A DNA nanoscope via auto-cycling proximity recording. Nature communications, 8(1), 696.
41) Boulgakov, A. A., Xiong, E., Bhadra, S., Ellington, A. D., & Marcotte, E. M. (2018). From Space to Sequence and Back Again: Iterative DNA Proximity Ligation and its Applications to DNA-Based Imaging. BioRxiv, 470211.
42) Weinstein, J. A., Regev, A., & Zhang, F. (2019). DNA microscopy: Optics-free spatio-genetic imaging by a stand-alone chemical reaction. Cell.
43) Zhu Y Y, Machleder E M, et al. (2001) Reverse transcriptase template switching: a SMART approach for full-length cDNA library construction Biotechniques, 30(4):892-897.
44) Chu, H., Zhao, J., Mi, Y., Zhao, Y., & Li, L. (2019). Near-infrared Light-Initiated Hybridization Chain Reaction for Spatially- and Temporally-Resolved Signal Amplification. Angewandte Chemie International Edition.
45) Singh-Gasson, S., Green, R. D., Yue, Y., Nelson, C., Blattner, F., Sussman, M. R., & Cerrina, F. (1999). Maskless fabrication of light-directed oligonucleotide microarrays using a digital micromirror array. Nature biotechnology, 17(10), 974.
46) Rosenberg, Alexander B., et al. “Single-cell profiling of the developing mouse brain and spinal cord with split-pool barcoding.” Science 360.6385 (2018): 176-182.
47) Hagemann-Jensen, Michael, et al. “Single-cell RNA counting at allele and isoform resolution using Smart-seq3.” Nature Biotechnology 38.6 (2020): 708-714.
48) Dobin, Alexander, et al. “STAR: ultrafast universal RNA-seq aligner.” Bioinformatics 29.1 (2013): 15-21.

What is claimed is:

1. A barcode composition comprising:
a. a first nucleic acid comprising in a 5′ to 3′ direction:
i. optionally, a unique molecule identifier (UMI) sequence;
ii. a first targeting domain; and
iii. a first hybridization domain, and
b. a second nucleic acid comprising in a 5′ to 3′ direction:
i. a barcode domain; and
ii. a second hybridization domain, wherein the second hybridization domain is substantially complementary to the first hybridization domain of the first nucleic acid, and
wherein at least one of the first or second hybridization domain comprises a photoreactive element.

2. The barcode composition of claim 1, wherein the second nucleic acid further comprises a unique molecule identifier sequence at 5′-end.

3. The barcode composition of claim 1 or 2, wherein the second nucleic acid further comprises a primer sequence at the 5′-end.

4. A barcode composition comprising:
a. a first nucleic acid comprising in a 5′ to 3′ direction:
i. optionally, a unique molecule identifier sequence;
ii. a first targeting domain; and
iii. a first hybridization domain; and
b. a second nucleic acid comprising in a 5′ to 3′ direction:
i. a second hybridization domain, wherein the second hybridization domain is substantially complementary to the first hybridization domain of the first nucleic acid; and
ii. a first barcode domain, and
wherein at least one of the first or second hybridization domain comprises a photoreactive element.

5. The barcode composition of any one of claims 1-4, further comprising a third nucleic acid comprising in a second barcode domain, wherein the second barcode domain is substantially complementary to the first barcode domain.

6. The barcode composition of claim 5, wherein the third nucleic acid further comprises a unique molecule identifier sequence at 5′-end.

7. The barcode composition of claim 5 or 6, wherein the third nucleic acid further comprises a primer sequence at the 5′-end.

8. A barcode composition comprising:
a. a first nucleic acid comprising in a 5′ to 3′ direction:
i. optionally, a unique molecule identifier sequence;
ii. a first targeting domain; and
iii. a first hybridization domain; and
b. a second nucleic acid comprising in a 5′ to 3′ direction:
i. a second hybridization domain, wherein the second hybridization domain is substantially complementary to the first hybridization domain of the first nucleic acid; and
ii. a first barcode domain;
iii. a third hybridization domain, and
wherein at least one of the first or second hybridization domains comprises a photoreactive element, and the third hybridization domains optionally comprises a photoreactive element.

9. The barcode composition of claim 8, wherein the composition further comprises n additional nucleic acids, wherein:
n is an integer from 1 to 100, and
each additional nucleic acid comprises in 5′ to 3′ direction:
i. a first hybridization domain;
ii. a barcode domain; and
iii. a second hybridization domain, and
wherein the first hybridization domain of nth nucleic acid is substantially complementary to the second hybridization domain of (n−1)th nucleic acid,
wherein the first hybridization domain of n=1 nucleic acid is substantially complementary to the third hybridization domain, and
and wherein at least one of the first or the second hybridization domain of each nucleic acid comprises a photoreactive element.

10. The barcode composition of claim 8 or 9, wherein the composition further comprises a first cap nucleic acid strand comprising in 5′ to 3′ direction:
i. a first cap hybridization domain, wherein the first cap hybridization domain is substantially complementary to the second hybridization domain of nth nucleic acid when n is 1 or more, or the cap hybridization domain is substantially complementary to the third hybridization domain when n is 0; and
ii. a second cap hybridization domain;
wherein the first cap hybridization domain optionally comprises a photoreactive element.

11. The barcode composition of claim 10, wherein the composition further comprises a second cap nucleic acid strand comprising in 5′ to 3′ direction:
i. a primer sequence domain;
ii. optionally, a unique molecular identifier (UMI) sequence; and
iii. a hybridization domain, wherein the hybridization domain is substantially complementary to the second cap hybridization domain of the first cap nucleic acid, and
wherein at least one of the second cap hybridization domain of the first cap nucleic acid strand and the hybridization domain of the second nucleic acid strand comprises a photoreactive element.

12. The barcode composition of any one of claims 1-11, wherein the first nucleic acid is an RNA or RNA transcript, and optionally, the first hybridization domain comprises a poly(A) sequence.

13. The barcode composition of any one of claims 1-12, wherein the first nucleic acid further comprises a primer sequence at the 5′-end.

14. The barcode composition of any one of claims 1-13, wherein the first targeting domain of the first nucleic acid is substantially complementary to a target nucleic acid.

15. The barcode composition of claim 14, wherein the target nucleic acid is conjugated with a target binding agent, or the target nucleic acid is conjugated with a target molecule, or the target nucleic acid is comprised within a target molecule (such as RNA), or the target nucleic acid is expressed by a target cell, or the target nucleic acid is presented on a target molecule or cell directly or indirectly via chemical crosslinking, genetic encoding, viral transduction, transfection, conjugation, cell fusion, cellular uptake, hybridization, DNA binding proteins or adaptor molecules such as target binding ligands.

16. The barcode composition of claim 15, wherein the target binding agent is selected from the group consisting of: amino acids, peptides, proteins, monosaccharides, disaccharides, trisaccharides, oligosaccharides, polysaccharides, lipopolysaccharides, lectins, nucleosides, nucleotides, nucleic acids, vitamins, steroids, hormones, cofactors, receptors and receptor ligands, optionally the target binding agent is an antibody or an antigen binding fragment thereof.

17. The barcode composition of any one of claims 1-16, wherein each domain independently comprises a 1 letter code, a 2 letter code, a 3 letter code, or a 4 letter code.

18. The barcode composition of any one of claims 1-17, wherein each domain independently comprises zero or at least one nucleic acid modifications.

19. The barcode composition of claim 18, wherein the nucleic acid modification is selected from the group consisting of nucleobase modifications, sugar modifications, and internucleotide linkage modifications.

20. The barcode composition of any one of claims 1-19, wherein each domain is independently 1-1000 nucleotides in length.

21. The barcode composition of any one of claims 1-20, wherein the UMI of a nucleic acid is incorporated into one of the other domains of the same nucleic acid.

22. The barcode composition of any one of claims 1-21, wherein at least one of the nucleic acid comprises a cleavable spacer.

23. The barcode composition of 22, wherein the cleavable spacer is a photocleavable spacer.

24. The barcode composition of any one of claims 1-23, wherein the composition further comprises a detectable label.

25. The barcode composition of claim 24, wherein the detectable label is comprised in one of the nucleic acids.

26. The barcode composition of claim 24 or 25, wherein the detectable label is selected from the group consisting of: fluorescent molecules, nanoparticles, stable isotopes, radioisotopes, nucleotide chromophores, enzymes, enzyme substrates, chemiluminescent moieties and bioluminescent moieties, echogenic substances, non-metallic isotopes, optical reporters, paramagnetic metal ions, and ferromagnetic metals, optionally the detectable label is a fluorophore.

27. The barcode composition of any one of claims 1-26, further comprising a polymerase.

28. The barcode composition of claim 27, wherein the polymerase is a strand-displacing polymerase.

29. The barcode composition of any one of claims 1-28, further comprising a buffer or salt for nucleic acid synthesis.

30. The barcode composition of any one of claims 1-29, further comprising natural or synthetic nucleotide triphosphates or deoxynucleotide triphosphates.

31. The barcode composition of any one of claims 1-30, further comprising a target element.

32. The barcode composition of claim 31, wherein the target element is immobilized on a substrate surface.

33. The barcode composition of claim 32, wherein the target element is immobilized on the substrate surface in a predetermined pattern.

34. The barcode composition of any one of claims 31-33, wherein the target element is a nucleic acid, a lipid, a sugar, a small molecule, a microorganism or fragment thereof, a polypeptide, and/or a biological material.

35. The barcode composition of claim 34, wherein the biological material is selected from the group consisting of: a tissue, a cell, an organoid, an engineered tissue; and an extracellular matrix.

36. The barcode composition of any one of claims 31-35, wherein the substrate is selected from the group consisting of: glass, transparent polymers, polystyrene, hydrogels, metal, ceramic, paper, agarose, gelatin, alginate, dextran, iron oxide, stainless steel, gold, copper, silver chloride, polycarbonate, polydimethylsiloxane, polyethylene, acrylonitrile butadiene styrene, cyclo-olefin polymers, cyclo-olefin copolymers, streptavidin, resin, and a biological material.

37. The barcode composition of any one of claims 1-36, wherein the photoreactive element is a photoreactive nucleotide, optionally the photoreactive nucleotide is a CNVK or a CNVD crosslinking base.

38. The barcode composition of any one of claims 1-37, further comprising PCR primers.

39. The barcode composition of any one of claims 1-38, further comprising a light source, optionally the light source is a UV light source.

40. The barcode composition of any one of claims 1-39 in form of a kit.

41. A method of detecting a target mRNA, the method comprising:
a. hybridizing a target mRNA (a first nucleic acid) with a second nucleic acid, and wherein:
i. the mRNA comprises a first hybridization domain comprising a polyA sequence; and
ii. the second nucleic acid comprises in a 5′ to 3′ direction:
1. a second hybridization domain, wherein the second hybridization domain is substantially complementary to the first hybridization domain and comprises a photoreactive element; and
2. a first barcode domain, and
b. photocrosslinking the mRNA with the second nucleic acid thereby forming a probe-primer complex;
c. synthesizing a record nucleic acid from the probe-primer complex; and
d. detecting the record nucleic acid.

42. A method of detecting a target nucleic, the method comprising:
a. hybridizing a target nucleic acid with a first nucleic acid and hybridizing a second nucleic acid with the first nucleic acid, wherein:
i. the first nucleic acid comprising in a 5′ to 3′ direction:
1. optionally, a unique molecule identifier (UMI) sequence;
2. a first targeting domain substantially complementary to a nucleic acid of the target element; and
3. a first hybridization domain; and
ii. the second nucleic acid comprising in a 5′ to 3′ direction:
1. a second hybridization domain, wherein the second hybridization domain is substantially complementary to the first hybridization domain; and
2. a first barcode domain, and
wherein at least one of the first or second hybridization domain comprises a photoreactive element;
b. photocrosslinking the first nucleic acid with the second nucleic acid thereby forming a probe-primer complex;
c. optionally, denaturing the probe-primer complex from the target nucleic acid;
d. synthesizing a record nucleic acid from the probe-primer complex; and
e. detecting the record nucleic acid.

43. The method of claim 41 or 42, wherein the second nucleic acid further comprises a unique molecule identifier (UMI) sequence at 5′-end.

44. The of method of any one of claims 41-43, wherein the second nucleic acid further comprises a primer sequence at 5′-end.

45. The method of any one of claims 41-44, wherein said detecting comprises sequencing the record nucleic acid, light microscopy, high throughput scanner, confocal microscopy, light sheet microscopy, electron microscopy, atomic force microscopy, or the unaided eye.

46. The method of claim 45, further comprising cleaving, uncrosslinking, removing or reversing the photocrosslink and amplifying the record nucleic acid prior to sequencing.

47. The method of claim 46, wherein said cleaving, uncrosslinking, removing, or reversing is using a 300-350 nm, optionally a 312 nm, wavelength of light.

48. A method of detecting a target mRNA, the method comprising:
a. hybridizing a target mRNA (a first nucleic acid) with a second nucleic acid, and wherein:
i. the mRNA comprises a first hybridization domain comprising a polyA sequence; and
ii. the second nucleic acid comprises in a 5′ to 3′ direction:
1. a second hybridization domain, wherein the second hybridization domain is substantially complementary to the first hybridization domain of the mRNA and comprises a photoreactive element; and
2. a first barcode domain, and
b. photocrosslinking the mRNA with the second nucleic acid thereby forming a first complex;
c. hybridizing a third nucleic acid to the second nucleic in the first complex thereby forming a probe-primer complex, wherein the third nucleic acid comprises a second barcode domain substantially complementary to the first barcode domain of the second nucleic acid;
d. synthesizing a record nucleic acid from the probe-primer complex; and
e. detecting the record nucleic acid.

49. A method of detecting a target nucleic acid, the method comprising:
a. hybridizing a target nucleic acid with a first nucleic acid and hybridizing a second nucleic acid to the first nucleic acid, wherein:
i. the first nucleic acid comprises in a 5′ to 3′ direction:
1. optionally, a unique molecule identifier (UMI) sequence;
2. a first targeting domain, wherein the first targeting domain is substantially complementary to the target nucleic acid; and
3. a first hybridization domain; and
ii. the second nucleic acid comprises in a 5′ to 3′ direction:
1. a second hybridization domain, wherein the second hybridization domain is substantially complementary to the first hybridization domain of the first nucleic acid;
2. a first barcode domain, and
wherein at least one of the first or second hybridization domain comprises a photoreactive element; and
b. photocrosslinking the first nucleic acid with the second nucleic acid thereby forming a first complex;
c. optionally, denaturing the first complex from the target nucleic acid;
d. hybridizing a third nucleic acid to the second nucleic acid in the first complex thereby forming a probe-primer complex, wherein the third nucleic acid comprises a second barcode domain substantially complementary to the first barcode domain of the second nucleic acid
e. synthesizing a record nucleic acid from the probe-primer complex; and
f. detecting the record nucleic acid.

50. The method of claim 48 or 49, wherein the third nucleic acid further comprises a unique molecule identifier (UMI) sequence at 5′-end.

51. The method of any one claims 48-50, wherein the third nucleic acid further comprises a primer sequence at 5′-end.

52. The method of any one of claims 48-51, wherein said detecting comprises sequencing the record nucleic acid, light microscopy, high throughput scanner, confocal microscopy, light sheet microscopy, electron microscopy, atomic force microscopy, or the unaided eye.

53. The method of claim 52, further comprising amplifying the record nucleic acid prior to sequencing.

54. A method of detecting a target nucleic acid, the method comprises:
a. hybridizing a target nucleic acid with a first nucleic acid, wherein:
i. the first nucleic acid comprises in a 5′ to 3′ direction:
1. optionally, a unique molecule identifier (UMI) sequence;
2. a first targeting domain, wherein the first targeting domain is substantially complementary to the target nucleic acid; and
3. a first hybridization domain;
b. preparing a concatemer by hybridizing n additional nucleic acids and photocrosslinking the additional nucleic acids with the first complex, wherein n is an integer from 1 to 100, and wherein each additional nucleic acid comprises in 5′ to 3′ direction:
i. a first hybridization domain;
ii. a barcode domain; and
iii. a second hybridization domain, and
wherein the first hybridization domain of nth nucleic acid is substantially complementary to the second hybridization domain of ¬(n−1)th nucleic acid, wherein the first hybridization domain of n=1 nucleic acid is substantially complementary to the first hybridization domain of the first nucleic acid, and wherein at least one of the first or second hybridization domain of each nucleic acid comprises a photoreactive element;
c. hybridizing a first cap nucleic acid strand with the concatemer thereby forming a capped concatemer, wherein the first cap nucleic acid comprises
i. a first cap hybridization domain, wherein the first cap hybridization domain is substantially complementary to the second hybridization domain of nth nucleic acid; and
ii. a second cap hybridization domain;
d. hybridizing a second cap nucleic acid strand to the capped concatemer, thereby forming a concatemer-primer complex, wherein the second cap nucleic acid strand comprises in a 5′ to 3′ direction:
i. a primer sequence domain;
ii. optionally, a unique molecular identifier (UMI) sequence; and
iii. a hybridization domain, wherein the hybridization domain is substantially complementary to the second cap hybridization domain of the first cap nucleic acid; and
e. detecting the concatemer-primer complex or synthesizing a record nucleic acid from the concatemer-primer complex and detecting the record nucleic acid.

55. The method of claim 54, wherein said detecting comprises sequencing the record nucleic acid, light microscopy, high throughput scanner, confocal microscopy, light sheet microscopy, electron microscopy, atomic force microscopy, or the unaided eye.

56. The method of claim 55, further comprising amplifying the record nucleic acid prior to sequencing.

57. The method of any one of claims 41-54, wherein the photocrosslinking is performed in aqueous solution.

58. The method of any one of claims 41-55, wherein said photocrosslinking is using a 350-400 nm, optionally a 365 nm, wavelength of light.

59. The method of any one of claims 41-58, further comprising one or more wash steps.

60. The method of any one of claims 41-59, wherein the target nucleic acid is conjugated with a target binding ligand.

61. The method of claim 60, wherein the target binding ligand is selected from the group consisting of amino acids, peptides, proteins, monosaccharides, disaccharides, trisaccharides, oligosaccharides, polysaccharides, lipopolysaccharides, lectins, nucleosides, nucleotides, nucleic acids, vitamins, steroids, hormones, cofactors, receptors and receptor ligands, optionally the target binding ligand is an antibody or an antigen binding fragment thereof.

62. The method of any one of claims 41-61, wherein the target nucleic acid is comprised in a biological material.

63. The method of claim 62, wherein the biological material is selected from the group consisting of: a tissue, a cell, an organoid, an engineered tissue, and an extracellular matrix.

64. The method of any one of claims 41-63, wherein the target nucleic acid is immobilized on a substrate surface.

65. The method of any one of claims 41-64, wherein the target nucleic acid is immobilized on a substrate surface in a predetermined pattern.

66. The method of claim 65, wherein the substrate is selected from the group consisting of: glass, transparent polymers, polystyrene, hydrogels, metal, ceramic, paper, agarose, gelatin, alginate, dextran, iron oxide, stainless steel, gold, copper, silver chloride, polycarbonate, polydimethylsiloxane, polyethylene, acrylonitrile butadiene styrene, cyclo-olefin polymers, cyclo-olefin copolymers, streptavidin, resin, and a biological material.

67. The method of any one of claims 41-66, wherein the first nucleic acid further comprises a primer sequence at the 5′-end.

68. The method of any one of claims 41-67, wherein each domain independently comprises a 1 letter code, a 2 letter code, a 3 letter code, or a 4 letter code.

69. The method of any one of claims 41-68, wherein each domain independently comprises zero or at least one nucleic acid modifications.

70. The method of claim 69, wherein the nucleic acid modification is selected from the group consisting of nucleobase modifications, sugar modifications, and internucleotide linkage modifications.

71. The method of any one of claims 41-70, wherein each domain is independently 1-1000 nucleotides in length.

72. The method of any one of claims 41-71, wherein the UMI of a nucleic acid is incorporated into the barcode domain or the probe domain the same nucleic acid.

73. The method of any one of claims 41-72, wherein at least one of the nucleic acid comprises a cleavable spacer.

74. The method of claim 73, wherein the cleavable spacer is a photocleavable spacer.

75. The method of any one of claims 41-74, wherein at least one of the nucleic acid comprises a detectable label.

76. The method of claim 75, wherein the detectable label is selected from the group consisting of fluorescent molecules, radioisotopes, nucleotide chromophores, enzymes, enzyme substrates, chemiluminescent moieties and bioluminescent moieties, echogenic substances, non-metallic isotopes, optical reporters, paramagnetic metal ions, and ferromagnetic metals, optionally the detectable label is a fluorophore.

77. The method of any one of claims 41-76, wherein said synthesizing the record nucleic acid comprises using a strand-displacing polymerase.

78. The method of any one of claims 41-77, further comprising selecting one or more specific regions of interest for illumination or detection.

79. The method of claim 78, wherein said selecting one or more specific regions is manual or computer aided.

80. The method of claim 78 or 79, wherein the selection is based on one or more phenotypic markers.

81. The method of claim 80, wherein the one or more phenotypic marker is fluorescence, shape, intensity, histological stains, antibody staining, or morphology.

82. The method of any one of claims 41-81, further comprising software that automatically detects one or more regions of interest for spatial illumination or detection.

83. A method for linearly, combinatorially or spatially barcoding a plurality of targets in a sample, the method comprising:
a. hybridizing a target nucleic acid strand in each member the plurality of targets with a first nucleic acid strand, wherein the target nucleic acid strand is different in each member the plurality of targets, wherein the target nucleic acid strand is comprised within another nucleic acid molecule, or the target nucleic acid strand is conjugated with a member of the plurality of targets, or the target nucleic acid strand is expressed by a cell, or the target nucleic acid strand is presented on a target or cell directly or indirectly via chemical crosslinking, genetic encoding, viral transduction, transfection, conjugation, cell fusion, cellular uptake, hybridization, DNA binding proteins or a target binding agent/ligand, and wherein:
i. the first nucleic acid strand comprises in a 5′ to 3′ direction:
1. optionally, a unique molecule identifier (UMI) sequence;
2. a first targeting domain, wherein the first targeting domain is substantially complementary to the target nucleic acid; and
3. a first hybridization domain;
b. preparing a concatemer by hybridizing in a stepwise manner one or more additional nucleic acid strand and photocrosslinking the additional nucleic acid strands with the first complex, wherein said photocrosslinking comprises selecting predetermined regions of the sample and exposing the predetermined regions to light after hybridizing each additional nucleic acid strand thereby cross-linking the complementary hybridization domains, and removing any non-crosslinked additional nucleic acid strands after exposure to light and prior to hybridization a next additional nucleic acid strand, and
wherein each additional nucleic acid strand comprises in 5′ to 3′ direction:
i. a first hybridization domain;
ii. a barcode domain; and
iii. a second hybridization domain, and
wherein the first hybridization domain of nth additional nucleic acid strand is substantially complementary to the second hybridization domain of (n−1)th additional nucleic acid strand, wherein the first hybridization domain of the first additional nucleic acid strand is substantially complementary to the first hybridization domain of the first nucleic acid strand, and wherein at least one of the first or second hybridization domain of each nucleic acid strand comprises a photoreactive element; and
c. detecting the concatemer and/or synthesizing a record nucleic acid from the concatemer and detecting the record nucleic acid.

84. The method of claim 83, wherein at least one member of plurality of targets is comprised within another nucleic acid molecule.

85. The method of claim 83 or 84, wherein at least one member of plurality of targets is comprised within another nucleic acid molecule selected independently from the group consisting of RNA, RNA transcript, genomic DNA, nucleic acid amplification products, and any combinations thereof.

86. The method of any one of claims 83-85, wherein at least one member of plurality of targets is a cDNA.

87. The method of any one of claims 83-86, wherein at least one member of plurality of targets is a non-nucleic acid molecule conjugated to the target nucleic acid stand.

88. The method of any one of claims 83-87, wherein at least one member of plurality of targets is a non-nucleic acid molecule conjugated to the target nucleic acid stand via a targeting binding agent linked to the target nucleic acid stand.

89. The method of any one of claims 83-88, wherein the target binding agent/ligand is selected from the group consisting of: amino acids, peptides, proteins, monosaccharides, disaccharides, trisaccharides, oligosaccharides, polysaccharides, lipopolysaccharides, lectins, nucleosides, nucleotides, nucleic acids, vitamins, steroids, hormones, cofactors, receptors and receptor ligands, optionally the target binding agent is an antibody or an antigen binding fragment thereof.

90. The method of any one of claims 83-89, wherein at least one member of the plurality of the targets is a nucleic acid and at least one member of the plurality of the targets is a non-nucleic acid molecule.

91. The method of any one claims 83-90, wherein at least one member of plurality of targets is a protein.

92. The method of any one of claims 83-91, wherein the sample is a biological material.

93. The method of any one of claims 83-92, wherein the sample is a biological material selected from the group consisting of: a tissue, a cell, an organoid, an engineered tissue, and an extracellular matrix.

94. The method of any one of claims 83-92, wherein the sample is selected from the group consisting of whole tissues, tissue regions, collection of cells, single cells, subcellular regions, and any combinations thereof.

95. The method of any one claims 83-94, wherein the photoreactive element is CNVK.

96. The method of any one of claims 83-95, wherein the photoreactive element inhibits or blocks activity of a polymerase, optionally, the polymerase is a strand-displacing polymerase.

97. The method of any one of claims 83-96, wherein the method comprises detecting the concatemer and/or record strand by an imaging method and sequencing the record nucleic acid for multimodal integrated analysis of predefined regions of the sample.

98. The method of any one of claims 83-97, wherein the method comprises detecting the concatemer and/or record strand by an imaging method and sequencing the record nucleic acid for correlating the sequence of the record strands to spatial positions for multimodal integrated analysis of predefined regions of the sample.

99. The method of any one of claims 83-98, wherein said detecting comprises sequencing the record nucleic acid, light microscopy, high throughput scanner, confocal microscopy, light sheet microscopy, electron microscopy, atomic force microscopy, or the unaided eye.

100. The method of claim 99, further comprising amplifying the record nucleic acid prior to sequencing.

101. The method of claim 100, further comprising cleaving, uncrosslinking, removing or reversing the photocrosslink and amplifying the record nucleic acid prior to sequencing.

102. The method of any one of claims 83-101, wherein said photocrosslinking is using a 350-400 nm, optionally a 365 nm, wavelength of light.

103. The method of any one of claims 83-102, wherein each domain independently comprises a 1 letter code, a 2 letter code, a 3 letter code, or a 4 letter code.

104. The method of any one of claims 83-103, wherein at least one of the nucleic acid strands comprises a detectable label.

105. The method of claim 104, wherein the detectable label is selected from the group consisting of fluorescent molecules, radioisotopes, nucleotide chromophores, enzymes, enzyme substrates, chemiluminescent moieties and bioluminescent moieties, echogenic substances, non-metallic isotopes, optical reporters, paramagnetic metal ions, and ferromagnetic metals, optionally the detectable label is a fluorophore.

106. The method of any one of claims 83-105, wherein said synthesizing the record nucleic acid comprises using a strand-displacing polymerase.

107. The method of any one of claims 83-106, wherein selecting the predetermined regions is manual or computer aided.

108. Use of a method of any one of claims 40-107 for screening a library of candidates for treatment, the use comprising identifying one or more phenotypic markers by imaging and barcoding predefined regions by a method of any one of claims 40-107.

109. The use of claim 108, wherein the one or more phenotypic marker is fluorescence, shape, intensity, histological stains, antibody staining, or morphology.

110. Use of a method of any one of claims 40-107 for identifying for screening of candidates, identification of drug targets, identification of biomarkers, profiling, characterization of phenotypic to genotypic cell state, generation of new disease models, characterization of cells and disease models, characterization of differentiation status and cell state, tissue mapping, multi-dimensional analysis, high content screening, machine-learning based clustering or classification, cell therapy development, CAR-T therapy development, antibody screening, personalized medicine, cell enrichment, and any combinations thereof.

111. The use of any one of claims 108-110, wherein the candidates are selected from the group consisting of small molecule drugs, biologics, therapeutic nucleic acids, gene or cell therapies, siRNAs, gRNAs, peptides, proteins, antibodies, metabolites, hormones, and DNA encoded libraries.

112. The kit of claim 40 for use in a method for barcoding biomolecules in vitro, in vivo, in situ or in toto using a method of any one of claims 83-111.
